SEQUENCE LISTING (99 sequences)

SEQ ID NO: 1 - protein, 107 aa, synthetic construct
DIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIK

SEQ ID NO: 2 - protein, 123 aa, synthetic construct
QVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSS

SEQ ID NO: 3 - protein, 249 aa, synthetic construct
DIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSS

SEQ ID NO: 4 - protein, 110 aa, synthetic construct
QAVVTQEPSFSVSPGGTVTLTCGSSTGAVTTSNYANWVQQTPGQAFRGLIGGTNKRSSGVPDRFSGSLLGDKAALTITGAQADDESDYYCALWYSNLWVFGGGTKLTVLG

SEQ ID NO: 5 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGNSYVSWFAYWGQGTLVTVSS

SEQ ID NO: 6 - protein, 110 aa, synthetic construct
QAVVTQEPSLTVSPGGTVTLTCGSSTGAVTTSNYANWVQQKPGQAFRTLIGGTNKKHPWTPARFSGSLLGGKAALTLSGAQPEDEAEYYCALWYSNLWVFGGGTKLTVLG

SEQ ID NO: 7 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKSTAYLQMNSLKTEDTAVYYCVRHGNFGNSYVSWFAYWGQGTLVTVSS

SEQ ID NO: 8 - protein, 216 aa, synthetic construct
QAVVTQEPSFSVSPGGTVTLTCGSSTGAVTTSNYANWVQQTPGQAFRGLIGGTNKRSSGVPDRFSGSLLGDKAALTITGAQADDESDYYCALWYSNLWVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 107 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIK

SEQ ID NO: 10 - protein, 123 aa, synthetic construct
QVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSS

SEQ ID NO: 11 - protein, 719 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGTSYVSWFAYWGQGTLVTVSS

SEQ ID NO: 13 - protein, 719 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGTSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 110 aa, synthetic construct
QAVVTQEPSFSVSPGGTVTLTCGSSTGAVTTSNYANWVQQTPGQAFRGLIGGTNKRVSGVPDRFSGSLLGDKAALTITGAQADDESDYYCALWFSNHWVFGGGTKLTVLG

SEQ ID NO: 15 - protein, 216 aa, synthetic construct
QAVVTQEPSFSVSPGGTVTLTCGSSTGAVTTSNYANWVQQTPGQAFRGLIGGTNKRVSGVPDRFSGSLLGDKAALTITGAQADDESDYYCALWFSNHWVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 110 aa, synthetic construct
QAVVTQEPSFSVSPGGTVTLTCGSSTGAVTTSNYANWVQQTPGQAFRGLIGGTNKRSSGVPDRFSGSLLGDKAALTITGAQADDESDYYCALWFSNHWVFGGGTKLTVLG

SEQ ID NO: 17 - protein, 216 aa, synthetic construct
QAVVTQEPSFSVSPGGTVTLTCGSSTGAVTTSNYANWVQQTPGQAFRGLIGGTNKRSSGVPDRFSGSLLGDKAALTITGAQADDESDYYCALWFSNHWVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGHFGTSYVSWFAYWGQGTLVTVSS

SEQ ID NO: 19 - protein, 719 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGHFGTSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGMFGTSYVSWFAYWGQGTLVTVSS

SEQ ID NO: 21 - protein, 719 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGMFGTSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGQFGTSYVSWFAYWGQGTLVTVSS

SEQ ID NO: 23 - protein, 719 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGQFGTSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 110 aa, synthetic construct
QAVVTQEPSFSVSPGGTVTLTCGSSTGAVTTSNYANWVQQTPGQAFRGLIGGTNKRVSGVPDRFSGSLLGDKAALTITGAQADDESDYYCALWYSNHWVFGGGTKLTVLG

SEQ ID NO: 25 - protein, 216 aa, synthetic construct
QAVVTQEPSFSVSPGGTVTLTCGSSTGAVTTSNYANWVQQTPGQAFRGLIGGTNKRVSGVPDRFSGSLLGDKAALTITGAQADDESDYYCALWYSNHWVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 15 aa, synthetic construct
GGGGSGGGGSGGGGS

SEQ ID NO: 27 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMAARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPG

SEQ ID NO: 28 - protein, 719 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMAARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPG

SEQ ID NO: 29 - protein, 110 aa, synthetic construct
QAVVTQEPSFSVSPGGTVTLTCGSSTGAVTTSNYANWVQQTPGQAFRGLIGGTNKRASGVPDRFSGSLLGDKAALTITGAQADDESDYYCALWYSNHWVFGGGTKLTVLG

SEQ ID NO: 30 - protein, 216 aa, synthetic construct
QAVVTQEPSFSVSPGGTVTLTCGSSTGAVTTSNYANWVQQTPGQAFRGLIGGTNKRASGVPDRFSGSLLGDKAALTITGAQADDESDYYCALWYSNHWVFGGGTKLTVLGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 6 aa, synthetic construct
QEISGY

SEQ ID NO: 32 - skipped

SEQ ID NO: 33 - protein, 9 aa, synthetic construct
LQYASYPFT

SEQ ID NO: 34 - protein, 10 aa, synthetic construct
GFSLSTSTMG

SEQ ID NO: 35 - protein, 7 aa, synthetic construct
ILWNDSK

SEQ ID NO: 36 - protein, 15 aa, synthetic construct
ARIVYYSTYVGYFDV

SEQ ID NO: 37 - protein, 9 aa, synthetic construct
LQYASYPLT

SEQ ID NO: 38 - protein, 10 aa, synthetic construct
GFSLSRSTMG

SEQ ID NO: 39 - protein, 7 aa, synthetic construct
IKWNDSK

SEQ ID NO: 40 - protein, 9 aa, synthetic construct
TGAVTTSNY

SEQ ID NO: 41 - skipped

SEQ ID NO: 42 - protein, 9 aa, synthetic construct
ALWYSNLWV

SEQ ID NO: 43 - protein, 8 aa, synthetic construct
GFTFNTYA

SEQ ID NO: 44 - protein, 10 aa, synthetic construct
IRSKYNNYAT

SEQ ID NO: 45 - protein, 16 aa, synthetic construct
VRHGNFGNSYVSWFAY

SEQ ID NO: 46 - protein, 9 aa, synthetic construct
ALWFSNHWV

SEQ ID NO: 47 - protein, 9 aa, synthetic construct
ALWYSNHWV

SEQ ID NO: 48 - protein, 16 aa, synthetic construct
VRHGNFGTSYVSWFAY

SEQ ID NO: 49 - protein, 16 aa, synthetic construct
VRHGHFGTSYVSWFAY

SEQ ID NO: 50 - protein, 16 aa, synthetic construct
VRHGMFGTSYVSWFAY

SEQ ID NO: 51 - protein, 16 aa, synthetic construct
VRHGQFGTSYVSWFAY

SEQ ID NO: 52 - protein, 250 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSS

SEQ ID NO: 53 - protein, 390 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGNSYVSWFAYWGQGTLVTVSS

SEQ ID NO: 54 - protein, 390 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGTSYVSWFAYWGQGTLVTVSS

SEQ ID NO: 55 - protein, 390 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGHFGTSYVSWFAYWGQGTLVTVSS

SEQ ID NO: 56 - protein, 390 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGMFGTSYVSWFAYWGQGTLVTVSS

SEQ ID NO: 57 - protein, 390 aa, synthetic construct
DIQMTQSPSSLSASVGDRVTITCRASQEISGYLSWYQQKPEKAPKSLIYAASTLHSGVPSRFSGSGSGTDFTLTISSLQPEDFATYYCLQYASYPLTFGQGTKLEIKGGGGSGGGGSGGGGSGGGGSQVTLKESGPALVKPTQTLTLTCTFSGFSLSRSTMGVGWIRQPPGKALEWLAHIKWNDSKYYNPALKSRLTISKDTSKNQVVLTMTNMDPVDTATYYCARIVYYSTYVGYFDVWGQGTLVTVSSGGGGSGGGGSGGGGSEVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGQFGTSYVSWFAYWGQGTLVTVSS

SEQ ID NO: 58 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 59 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 20 aa, synthetic construct
GGGGSGGGGSGGGGSGGGGS

SEQ ID NO: 61 - protein, 19 aa, synthetic construct
GGGGSGGGGSGGGSGGGGS

SEQ ID NO: 62 - protein, 119 aa, synthetic construct
QVQLVQSGSELKKPGASVKVSCKASGYTFTSYWMHWVRQAPGQGLEWMGEIDPSDSYTTYNQGFTGRFVFSVDKSVSTAYLQISSLKAEDTAVYYCARSAYYSKRDDYWGQGTTVTVSS

SEQ ID NO: 63 - protein, 111 aa, synthetic construct
EIVLTQSPATLSLSPGERATLSCRASESVDNYGISFMNWFQQKPGQAPRLLIYAASNRATGIPARFSGSGPGTDFTLTISSLEPEDFAVYYCQQSKEVPWTFGQGTKLEIK

SEQ ID NO: 64 - protein, 119 aa, synthetic construct
EVQLVQSGAEVKKPGESLKISCKASGYTFTSYWMHWVRQMPGKGLEWMGEIDPSDSYTRYSQSFQGQVTISVDKSISTAYLQWSSLKASDTAMYYCARSAYYSKRDDYWGQGTTVTVSS

SEQ ID NO: 65 - protein, 111 aa, synthetic construct; identical to SEQ ID NO: 63

SEQ ID NO: 66 - protein, 259 aa, synthetic construct
EIVMTQSPGTLSLSPGERATLSCRASQEISGYLSWLQQKPGQAIRRLIYAASTRATGIPDRFSGSRSGSDYTLTISRLEPEDFAVYYCLQYASYPFTFGQGTKLEIKGGGGSGGGGSGGGSGGGGSQITLKESGPTLVKPTQTLTLTCTFSGFSLSTSTMGVGWIRQPPGKALEWLAHILWNDDKRYGPSLKSRLTITKDTSKKQVVLTMTNMDPVDTATYYCARIVYYSTYVGYFDVWGQGTTVTVSSHHHHHHHHHH

SEQ ID NO: 67 - protein, 125 aa, synthetic construct; identical to SEQ ID NO: 7

SEQ ID NO: 68 - protein, 110 aa, synthetic construct; identical to SEQ ID NO: 6

SEQ ID NO: 69 - protein, 125 aa, synthetic construct; identical to SEQ ID NO: 5

SEQ ID NO: 70 - protein, 110 aa, synthetic construct; identical to SEQ ID NO: 4

SEQ ID NO: 71 - protein, 11 aa, synthetic construct
RASQEISGYLS

SEQ ID NO: 72 - protein, 7 aa, synthetic construct
AASTLHS

SEQ ID NO: 73 - protein, 12 aa, synthetic construct
GFSLSRSTMGVG

SEQ ID NO: 74 - protein, 16 aa, synthetic construct
HIKWNDSKYYNPALKS

SEQ ID NO: 75 - protein, 13 aa, synthetic construct
IVYYSTYVGYFDV

SEQ ID NO: 76 - protein, 14 aa, synthetic construct
GSSTGAVTTSNYAN

SEQ ID NO: 77 - protein, 7 aa, synthetic construct
GTNKRSS

SEQ ID NO: 78 - protein, 7 aa, synthetic construct
GTNKRVS

SEQ ID NO: 79 - protein, 7 aa, synthetic construct
GTNKRSS

SEQ ID NO: 80 - protein, 7 aa, synthetic construct
GTNKRAS

SEQ ID NO: 81 - protein, 10 aa, synthetic construct
GFTFNTYAMN

SEQ ID NO: 82 - protein, 19 aa, synthetic construct
RIRSKYNNYATYYADSVKG

SEQ ID NO: 83 - protein, 14 aa, synthetic construct
HGNFGNSYVSWFAY

SEQ ID NO: 84 - protein, 14 aa, synthetic construct
HGNFGTSYVSWFAY

SEQ ID NO: 85 - protein, 14 aa, synthetic construct
HGHFGTSYVSWFAY

SEQ ID NO: 86 - protein, 14 aa, synthetic construct
HGMFGTSYVSWFAY

SEQ ID NO: 87 - protein, 14 aa, synthetic construct
HGQFGTSYVSWFAY

SEQ ID NO: 88 - protein, 107 aa, synthetic construct
EIVMTQSPGTLSLSPGERATLSCRASQEISGYLSWLQQKPGQAIRRLIYAASTRATGIPDRFSGSRSGSDYTLTISRLEPEDFAVYYCLQYASYPFTFGQGTKLEIK

SEQ ID NO: 89 - protein, 123 aa, synthetic construct
QITLKESGPTLVKPTQTLTLTCTFSGFSLSTSTMGVGWIRQPPGKALEWLAHILWNDDKRYGPSLKSRLTITKDTSKKQVVLTMTNMDPVDTATYYCARIVYYSTYVGYFDVWGQGTTVTVSS

SEQ ID NO: 90 - protein, 107 aa, synthetic construct
ETVLTQSPATLSVSPGERATLSCRASQSISNNLHWYQQKPGQAPRLLIKYGFQRATGIPARFSGSGSGTEFTLTISSLQSEDFAVYYCQQTNSWPLTFGQGTKLEIK

SEQ ID NO: 91 - protein, 115 aa, synthetic construct
QIQLVQSGAEVKKPGASVKVSCKASGYSFIDYNMYWVRQAPGQGLEWMGYINPYNGGTSYNQKFQGRVTMTVDKSTSTVYMELSSLRSEDTAVYYCARGTTGDYWGQGTLVTVSS

SEQ ID NO: 92 - protein, 107 aa, synthetic construct
ETVLTQSPATLSVSPGERATLSCRASQSISNNLHWYQQKPGQAPRLLIKYGFQRISGIPARFSGSGSGTEFTLTISSLQSEDFAVYYCQQTNSWPLTFGQGTKLEIK

SEQ ID NO: 93 - protein, 115 aa, synthetic construct
QIQLVQSGAEVKKPGASVKVSCKASGYSFIDYNMYWVRQAPGQGLEWMGYINPYNGGTSYNQKFQGRVTMTVDTSTSTVYMELSSLRSEDTAVYYCARGTTGDYWGQGTLVTVSS

SEQ ID NO: 94 - protein, 21 aa, Homo sapiens
ALRPQSSGTVYEAAAVEVDVS

SEQ ID NO: 95 - protein, 9 aa, Homo sapiens
MTETQAGEY

SEQ ID NO: 96 - protein, 9 aa, Homo sapiens
FTVSIRNTL

SEQ ID NO: 97 - protein, 119 aa, synthetic construct
QVQLVQSGAEVKKPGSSVKVSCKASGYTFTSYWMHWVRQAPGQGLEWMGEIDPSDSYTNYNQKFKGRVTITADESTSTAYMELSSLRSEDTAVYYCARSAYYSKRDDYWGQGTLVTVSS

SEQ ID NO: 98 - protein, 111 aa, synthetic construct
EIVLTQSPATLSLSPGERATLSCRASESVDNYGISFMNWYQQKPGQAPRLLIYAASEQGSGIPARFSGSGSGTDFTLTISSLEPEDFAVYYCWQSKEVPWTFGQGTKLEIK

SEQ ID NO: 99 - protein, 522 aa, Homo sapiens
NQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNDIEGRMD

(19) United States
(12) Patent Application Publication
(10) Pub. No.: US 2024/0368288 A1
(43) Pub. Date: Nov. 7, 2024

(54) BISPECIFIC ANTI-FLT3/CD3 ANTIBODIES AND METHODS OF USE

(71) Applicants: Hemogenyx Pharmaceuticals LLC, Brooklyn, NY (US); Eli Lilly and Company, Indianapolis, IN (US)

(72) Inventors: Vladislav M. SANDLER, New York, NY (US); Elina SHRESTHA, Long Island City, NY (US); Carina Rachel SIROCHINSKY, New York, NY (US); Raymond LIANG, Long Island City, NY (US); Ronen BEN JEHUDA, New York, NY (US); Andrew Ihor KORYTKO, Oceanside, CA (US); Andrew Dixon SKORA, Solana Beach, CA (US); Max Ephraim ADER, San Diego, CA (US); Todd Christopher CHILTON, San Diego, CA (US)

(21) Appl. No.: 18/684,097

(22) PCT Filed: Aug. 15, 2022
(86) PCT No.: PCT/US2022/074981

§ 371 Date: Feb. 15, 2024

Related U.S. Application Data
(60) Provisional application No. 63/329,138, filed on Apr. 8, 2022.
(60) Provisional application No. 63/234,226, filed on Aug. 17, 2021.

(51) Int. Cl. C07K 16/28; A61K 39/00; A61P 35/02

(52) U.S. Cl. CPC C07K 16/2863; A61P 35/02; C07K 16/2809; C07K 16/2818; A61K 2039/505; A61K 2039/545; C07K 2317/24; C07K 2317/31; C07K 2317/565; C07K 2317/622

(57) ABSTRACT

Provided herein are bispecific anti-FLT3/CD3 antibodies and antigen binding fragments thereof, such as the antibodies and fragments that specifically bind human FLT3 and human CD3. In some aspects, provided herein are optimized humanized, bispecific anti-FLT3/CD3 antibodies and antigen binding fragments thereof, optionally, having certain amino acid substitutions that confer advantageous properties (e.g., optimal antigen binding, manufacturability and/or half-life properties). Also provided herein are pharmaceutical compositions comprising such antibodies or fragments. Also provided herein are methods of use of such antibodies and fragments.

### CROSS-REFERENCE TO RELATED APPLICATIONS

This application claims the benefit of U.S. Provisional Patent Application No. 63/234,226 filed Aug. 17, 2021, and U.S. Provisional Patent Application No. 63/329,138 filed Apr. 8, 2022, each of which is incorporated by reference herein in its entirety.

### REFERENCE TO AN ELECTRONIC SEQUENCE LISTING

The contents of the electronic sequence listing (HEPH_002_002WO_SeqList_ST26.xml; Size: 96,828 bytes; and Date of Creation: Aug. 11, 2022) are incorporated by reference herein in their entirety.

### FIELD

In some aspects, the present invention relates to bispecific antibodies or antigen binding fragments thereof that bind to FLT3 and CD3, and uses of such antibodies.

### BACKGROUND

### FLT3

FLT3 is Fms Related Receptor Tyrosine Kinase 3. FLT3 is also known as fetal liver kinase 2 (FLK2). FLT3, a member of the class III tyrosine kinase receptor family, is expressed in normal hematopoietic progenitors as well as in leukemic blasts, and it plays an important role in cell proliferation, differentiation, and survival. Activation of the FLT3 receptor by the FLT3 ligand leads to receptor dimerization and phosphorylation, and activation of downstream signaling pathways, including the Janus kinase (JAK) 2 signal transducer (JAK2), signal transducer and activator of transcription (STAT) 5, and mitogen-activated protein kinase (MAPK) pathways. Mutations in the FLT3 gene, found in approximately 40% of patients with AML, are believed to promote its autophosphorylation and constitutive activation, leading to ligand-independent proliferation (Frankfurt O) et al., Current Opinion in Oncology (2007) 19 (6): 635-649).

Normal FLT3 expression is mostly restricted to CD34+ hematopoietic stem cells (HSCs), early hematopoietic progenitors (HPs), and dendritic cells (DCs). Activation of FLT3, through binding of FLT3 ligand (FLT3L), promotes normal differentiation of downstream blood lineages.

FLT3 expression is high in a variety of hematologic malignancies, including in most of AML patients. AML blasts in a majority of patients having AML express FLT3 and this expression is thought to promote survival and proliferation. Tyrosine kinase inhibitors (TKIs) have been developed to specifically target FLT3; however, secondary mutations leading to resistance against FLT3 remain a major obstacle.

### CD3

CD3 is cluster of differentiation 3 (a T cell co-receptor). CD3 is generally expressed on the membrane surface of mature T cells and activates naïve populations of T cells. The CD3 co-receptor helps activate cytotoxic and T helper cells (i.e. CD8+ T cells and CD4+ T cells). CD3 is a protein complex composed of four distinct chains. In mammals, the complex contains a CD3γ chain, a CD3δ chain, and two CD3ε chains, which associate with the T cell receptor (TCR) and the ζ chain to generate an activation signal in T lymphocytes. Together, the TCR, the ζ-chain and CD3 molecules comprise the TCR complex. The intracellular tail of CD3 contains a conserved motif known as the immunoreceptor tyrosine-based activation motif (ITAM), which is essential for the signaling capacity of the TCR. Upon phosphorylation of the ITAM, the CD3 chain can bind ZAP70 (zeta associated protein), a kinase involved in the signaling cascade of the T cell. Once activated, the T cells secrete cytokines and rapidly assist in the immune response.

### Hematopoietic Stem Cells

The hematopoietic stem cell is the common ancestor of all blood cells. As multipotent cells, they can differentiate into multiple cell lineages, but not all the lineages derived from the three germ layers. Hematopoietic stem cell differentiation gives rise to the lymphoid and myeloid cell lineages, the two major branches of hematopoiesis. (Kondo, M. “Lymphoid and myeloid lineage commitment in multipotent hematopoietic progenitors,” Immunol. Rev. 2010 November; 238 (1): 37-46). Lymphoid lineage cells include T, B, and natural killer (NK) cells. The myeloid lineage includes megakaryocytes and erythrocytes (MegE) as well as different subsets of granulocytes (neutrophils, eosinophils and basophils), monocytes, macrophages, and mast cells (GM), which belong to the myeloid lineage (Id. citing Kondo M, et al. Biology of hematopoietic stem cells and progenitors: implications for clinical application. Ann. Rev Immunol. 2003; 21:759-806, Weissman IL. Translating stem and progenitor cell biology to the clinic: barriers and opportunities. Science (New York, NY. 2000 Feb. 25;287 (5457): 1442-6); see also Iwaskaki, H. and Akashi, K. “Myeloid lineage commitment from the hematopoietic stem cell,” Immunity 26 (6) June 2007, 726-40).

HSCs present self-renewal potential and differentiation capacity into blood lineages; i.e., when stem cells divide, 50% of the daughter cells, on average, are committed with a cell lineage, while the remaining 50% do not differentiate. The process maintains the same number of stem cells by asymmetric cell division, so that each dividing stem cell originates one new stem cell and one differentiated cell. In contrast, in symmetric division, the stem cells originate 100% of identical stem cells. (Gordon, M. Stem cells and haemopoiesis. In: Hoffbrand, V., Catovsky, D., Tuddenham, E. G., 5th ed. Blackwell Publishing, (2005): Differential niche and Wnt requirements during acute myeloid leukemia, pp. 1-12. New York).

The lymphoid and myeloid lineages are separable at the progenitor level. Common lymphoid progenitors (CLPs) can differentiate into all types of lymphocytes without noticeable myeloid potential under physiological conditions (Kondo M, Scherer D C, Miyamoto T, King A G, Akashi K, Sugamura K. et al. Cell-fate conversion of lymphoid committed progenitors by instructive actions of cytokines. Nature. 2000 Sep. 21;407 (6802): 383-6), although some myeloid related genes might be detected in CLPs, depending on the experimental conditions (Delogu A, Schebesta A, Sun Q, Aschenbrenner K, Perlot T, Busslinger M. Gene repression by Pax5 in B cells is essential for blood cell homeostasis and is reversed in plasma cells. Immunity. 2006 March;24 (3): 269-81).

Similarly, common myeloid progenitors (CMPs) can give rise to all classes of myeloid cells with no or extensively low levels of B-cell potential (Akashi K, Traver D, Miyamoto T, Weissman IL. A clonogenic common myeloid progenitor that gives rise to all myeloid lineages. Nature. 2000 Mar. 9;404 (6774): 193-7). Another cell type, dendritic cells (DCs), is not clearly grouped either in lymphoid or myeloid lineage, because DC can arise from either CLPs or CMPs (Manz M G, Traver D, Miyamoto T, Weissman I L, Akashi K. Dendritic cell potentials of early lymphoid and myeloid progenitors. Blood. 2001 Jun. 1;97 (11): 3333-41, Traver D, Akashi K, Manz M, Merad M, Miyamoto T, Engleman E G, et al. Development of CD8alpha-positive dendritic cells from a common myeloid progenitor. Science (New York, NY. 2000 Dec. 15;290 (5499): 2152-4)). CMPs can proliferate and differentiate into megakaryocyte-erythrocyte (MegE) progenitors and granulocyte-monocyte (GM) progenitors, which further give rise to megakaryocytes, erythrocytes, granulocytes, monocytes and others. (Iwasaki H, Akashi K. Myeloid lineage commitment from the hematopoietic stem cell. Immunity. 2007; 26:726-740).

It is likely that differences in the expression levels of transcription factors determine the lineage affiliation of a differentiating cell. The transcription factors PU. 1 and GATA-1 have been implicated in myeloid and erythroid/megakaryocyte lineage differentiation, respectively (Gordon, M. Stem cells and haemopoiesis. In: Hoffbrand, V., Catovsky, D., Tuddenham, E. G., 5th ed. Blackwell Publishing, (2005): Differential niche and Wnt requirements during acute myeloid leukemia, pp. 1-12. New York).

### Characterization of HSCs

HSCs are undifferentiated and resemble small lymphocytes. A large fraction of HSCs is quiescent, in the G0 phase of the cell cycle, which protects them from the action of cell cycle-dependent drugs. The quiescent state of stem cells is maintained by transforming growth factor-β (TGF-β). The activity of TGF-β is mediated by p53, a tumor suppressor gene that regulates cell proliferation and targets the cyclin-dependent kinase inhibitor p21 (Gordon, M. Stem cells and haemopoiesis. In: Hoffbrand, V., Catovsky, D., Tuddenham, E. G., 5th ed. Blackwell Publishing, (2005): Differential niche and Wnt requirements during acute myeloid leukemia, pp. 1-12. New York). Quiescence of HSCs is critical not only for protecting the stem cell compartment and sustaining stem cell pools during long periods of time, but also for minimizing the accumulation of replication associated mutations. Many of the intrinsic transcriptional factors that maintain HSCs quiescence are found to be associated with leukemias. For example, chromosomal translocations resulting in the fusion of FoxOs and myeloid/lymphoid or mixed lineage leukemia have been reported in acute myeloid leukemias (See, e.g., Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1).

The majority of normal HSCs are present among the CD34+/CD38−/CD90+ bone marrow cell fractions with some HSCs also observed among CD34−/Lin− cells. CD34+/CD38+ cell fractions contain some HSCs endowed with short-term repopulating activity. Other recognized markers include the tyrosine kinase receptor c-kit (CD117) coupled with a lack of terminal differentiation markers such as CD4 and CD8 (Rossi et al., Methods in Molecular Biology (2011) 750 (2): 47-59).

### Classification of HSCs

The hematopoietic stem cell pool can be subdivided into three main groups: (1) short-term HSCs, capable of generating clones of differentiating cells for only 4-6 weeks; (2) intermediate-term HSCs, capable of sustaining a differentiating cell progeny for 6-8 months before becoming extinct; and (3) long-term HSCs, capable of maintaining hematopoiesis indefinitely. (Testa U. Annals of Hematology (2011) 90 (3): 245-271)

### Hematopoiesis

Hematopoiesis is a highly coordinated process wherein HSCs differentiate into mature blood cells supported by a specialized regulatory microenvironment, consisting of components which control the fate specification of stem and progenitor cells, as well as maintaining their development by supplying the requisite factors (“niche”). The term “bone marrow (BM) niche” as used herein refers to a well-organized architecture composed of elements (e.g., osteoblasts, osteoclasts, bone marrow endothelial cells, stromal cells, adipocytes and extracellular matrix proteins (ECM)) that play an essential role in the survival, growth and differentiation of diverse lineages of blood cells. The bone marrow niche is an important post-natal microenvironment in which HSCs proliferate, mature and give rise to myeloid and lymphoid progenitors.

Bone marrow (BM) is present in the medullary cavities of all animal bones. It consists of a variety of precursor and mature cell types, including hematopoietic cells (the precursors of mature blood cells) and stromal cells (the precursors of a broad spectrum of connective tissue cells), both of which appear to be capable of differentiating into other cell types. The mononuclear fraction of bone marrow contains stromal cells, hematopoietic precursors, and endothelial precursors.

Unlike secondary lymphoid organs such as spleen with distinct gross structures including red and white pulp, BM has no clear structural features, except for the endosteum that contains osteoblasts. The endosteum region comes in contact with calcified hard bones and provides a special microenvironment which is necessary for the maintenance of HSC activity (Kondo M, Immunology Reviews (2010) 238 (1): 37-46; Bydlowski and de Lara Janz (2012)). Hematopoietic Stem Cell in Acute Myeloid Leukemia Development, Advances in Hematopoietic Stem Cell Research, Dr. Rosana Pelayo (Ed.), ISBN: 978-953-307-930-1).

Within the niche, HSCs are believed to receive support and growth signals originating from several sources, including: fibroblasts, endothelial and reticular cells, adipocytes, osteoblasts and mesenchymal stem cells (MSCs). The main function of the niche is to integrate local changes in nutrients, oxygen, paracrine and autocrine signals and to change HSCs quiescence, trafficking, and/or expansion in response to signals from the systemic circulation (Broner, F. & Carson, MC. Topics in bone biology. Springer. 2009; 4: pp. 2-4. New York, USA.)

Although the nature of true MSCs remains misunderstood, CXC chemokine ligand 12 (CXCL12)-expressing CD146 MSCs were recently reported to be self-renewing progenitors that reside on the sinusoidal surfaces and contribute to organization of the sinusoidal wall structure, produce angiopoietin-1 (Ang-1), and are capable of generating osteoblasts that form the endosteal niche (Konopleva, M Y, & Jordan, C T, Biology and Therapeutic Targeting (2011) 9 (5): 591-599). These CXCL12 reticular cells may serve as a transit pathway for shuttling HSCs between the osteoblastic and vascular niches where essential but different maintenance signals are provided.

Cytokines and chemokines produced by bone marrow MSCs concentrate in particular niches secondary to varying local production and through the effects of cytokine binding glycosaminoglycans. Of these, CXCL12/stromal cell-derived factor-1 alpha positively regulates HSCs homing, while transforming growth factors FMS-like tyrosine kinase 3 (Flt3) ligand and Ang-1 act as quiescence factors (See, e.g., Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1). CXCL12-CXCR4 signaling is involved in homing of HSCs into BM during ontogeny as well as survival and proliferation of colony-forming progenitor cells. The CXCR4-selective antagonist-induced mobilization of HSCs into the peripheral blood further indicates a role for CXCL12 in retaining HSCs in hematopoietic organs. BM engraftment involves subsequent cell-to-cell interactions through the BMSC-produced complex extracellular matrix. Thus, vascular cell adhesion molecule-1 (VCAM-1) or fibronectin is critical for adhesion to the BM derived MSCs. In this way, the control of hematopoietic stem cell proliferation kinetics is critically important for the regulation of correct hematopoietic cell production. These control mechanisms could be classified as intrinsic or extrinsic to the stem cells, or a combination of both (See, e.g., Sergio Paulo Bydlowski and Felipe de Lara Janz (2012). Hematopoietic Stem Cell in Acute Myeloid Leukemia Development, Advances in Hematopoietic Stem Cell Research, Dr Rosana Pelayo (Ed.), ISBN: 978-953-307-930-1).

HSC self-renewal and differentiation can be controlled by external factors (extrinsic control), such as cell-cell interactions in the hematopoietic microenvironment or cytokines, such as SCF (stem cell factor) and its receptor c-kit, Flt-3 ligand, TGF-β, TNF-α and others. Cytokines regulate a variety of hematopoietic cell functions through the activation of multiple signal transduction pathways. The major pathways relevant to cell proliferation and differentiation are the Janus kinase (Jak)/signal transducers and activators of transcription (STATs), the mitogen-activated protein (MAP) kinase and the phosphatidylinositol (PI) 3-kinase pathways (Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1).

In addition, expression of other transcription factors, such as, stem cell leukemia (SCL) hematopoietic transcription factor; GATA-2; and gene products involved in cell cycle control, such as the cyclin dependent kinase inhibitors (CKIs) p16, p21 and p27 have been shown to be essential for hematopoietic cell development from the earliest stages (intrinsic control), (Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1).

Notch-1-Jagged pathway may serve to integrate extracellular signals with intracellular signaling and cell cycle control. Notch-1 is a surface receptor on hematopoietic stem cell membranes that binds to its ligand. Jagged, on stromal cells. This results in cleavage of the cytoplasmic portion of Notch-1, which can then act as a transcription factor (Gordon, M. Stem cells and haemopoiesis. In: Hoffbrand, V., Catovsky, D., Tuddenham, E. G., 5th ed. Blackwell Publishing, (2005): Differential niche and Wnt requirements during acute myeloid leukemia, pp. 1-12. New York).

Disorders that are Treated Using BM/HSC Transplantation

Disorders that are treated using Bone Marrow (BM)/Hematopoietic Stem Cell (HSC) transplantation include, without limitation, Acute Myeloid Leukemia (AML), Acute Lymphoblastic Leukemia (ALL), Chronic Lymphocytic Leukemia (CLL), Chronic Myeloid Leukemia (CML), Blastic plasmacytoid dendritic cell neoplasm (BPDCN), peripheral T cell lymphoma, follicular lymphoma, diffuse large B cell lymphoma, Hodgkin lymphoma, non-Hodgkin lymphoma, neuroblastoma, non-malignant inherited and acquired marrow disorders (e.g. sickle cell anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, or congenital thrombocytopenia), multiple myeloma, and Severe Combined Immunodeficiency (SCID).

### Hematopoietic Malignancies

Most hematopoietic malignancies comprise functionally heterogeneous cells, with only a subset, known as cancer stem cells, responsible for tumor maintenance. Cancer stem cells are so named because they possess qualities reminiscent of normal tissue stem cells including self-renewal, prolonged survival, and the ability to give rise to cells with more differentiated characteristics (Jones R J and Armstrong S A, Biol Blood Marrow Transplant. 2008 January; 14 (Supplement 1): 12-16).

A transforming event in hematopoietic stem cells can produce several different malignancies, including, without limitation, chronic myeloid leukemia, myelodysplastic syndrome, acute myeloid leukemia, and probably even acute lymphocytic leukemia, depending on the degree of differentiation associated with the oncogenic hit (Jones R J and Armstrong S A, Biol Blood Marrow Transplant. 2008 January; 14 (Supplement 1): 12-16).

The cancer stem cell concept is based on the idea that tumors of a specific tissue often appear to “attempt” to recapitulate the cellular heterogeneity found in the tissues of origin, and thus there are cells in the tumor that are stem-cell like giving rise to the varied cell types. A fundamental test for this hypothesis is whether tumor cells can be separated into those that have the ability to regenerate the tumor, and those that do not possess this ability. This cellular hierarchy has been most clearly demonstrated in acute myelogenous leukemias where some AMLs possess cells with a unique immunophenotype that are able to initiate leukemias in immunodeficient mice whereas most cells are unable to initiate leukemia development. Furthermore, the cells that initiate leukemias also give rise to cells that have lost tumor-initiating activity and thus recapitulate the cellular heterogeneity found in the original tumor (Lapidot T et al., Nature. 1994; 367:645-648; Bonnet D et al., Nat Med. 1997; 3:730-737).

### Acute Myeloid Leukemia

Acute myeloid leukemia (AML) is a clonal disorder characterized by arrest of differentiation in the myeloid lineage coupled with an accumulation of immature progenitors in the bone marrow, resulting in hematopoietic failure (Poll yea D A et al., British Journal of Haematology (2011) 152 (5): 523-542). There is wide patient-to-patient heterogeneity in the appearance of the leukemic blasts. The discovery of leukemia-initiating cells in acute myeloid leukemias (AMLs) started with the discovery that the large majority of AML blasts do not proliferate and only a small minority is capable of forming new colonies (Testa U, Annals of Hematology (2011) 90 (3): 245-271). A common feature to all AML cases is the arrested aberrant differentiation leading to an accumulation of more than 20% blast cells in the bone marrow (Gilliland, D G and Tallman M S, Cancer Cell (2002) 1 (5): 417-420).

More than 80% of myeloid leukemias are associated with at least one chromosomal rearrangement (Pandolfi P P, Oncogene (2001) 20 (40): 5726-5735), and over 100 different chromosomal translocations have been cloned (Gilliland, D G and Tallman M S, Cancer Cell (2002) 1 (5): 417-420). These translocations frequently involve genes encoding transcription factors that have been shown to play an important role in hematopoietic lineage development. Thus, alteration of the transcriptional machinery appears to be a common mechanism leading to arrested differentiation (Pandolfi P P, Oncogene (2001) 20 (40): 5726-5735; Tenen D G, Nature Reviews of Cancer (2003) 3 (2): 89-101).

Clinical investigation and experimental animal models suggest that at least two genetic alterations are required for the clinical manifestation of acute leukemia. According to the model proposed by Gilliland & Tallman (Cancer Cell (2002) 1 (5): 417-420), cooperation between class I activating mutations and class II mutations that induce termination of differentiation give rise to AML. The class I mutations, such as mutations in the receptor tyrosine kinase genes FLT3 and KIT, RAS family members, and loss of function of neurofibromin 1, confer proliferative and/or survival advantage to hematopoietic progenitors, typically as a consequence of aberrant activation of signal transduction pathways. The class II mutations lead to a halt in differentiation via interference with transcription factors or co-activators (Frankfurt O et al., Current Opinion in Oncology (2007) 19 (6): 635-649). While the leukemia stem cell (LSC) appears to share many of the cell surface markers previously identified for HSC such as CD34, CD38, HLA-DR, and CD71, several groups have reported surface markers that are differentially expressed in the two populations. For example, CD90 or Thy-1 has been described as potentially specific of the LSC compartment. Thy-1 is downregulated in normal hematopoiesis as the most primitive stem cells progress toward the progenitor stage. (Hope K J et al., Archives of Medical Research (2003) 34 (6): 507-514).

The interaction between CXCL12 (stromal cell-derived factor-1 alpha) and its receptor CXCR4 on leukemic progenitor cells contributes to their homing to the bone marrow microenvironment. CXCR4 levels are significantly elevated in leukemic cells from patients with AML, and CXCR4 expression is associated with poor outcome (Konopleva M Y and Jordan C T, Biology and Therapeutic Targeting (2011) 29 (5): 591-599).

Constitutive activation of the nuclear factor kappa β (NF-kβ) pathway I primary human AML stem cells provided evidence that NF-kβ plays a significant role in the overall survival of LSCs as well as AML cell types in general. (Konopleva M Y and Jordan C T, Biology and Therapeutic Targeting (2011) 29 (5): 591-599).

AML patients have poor clinical prognosis and limited therapeutic options, with myeloablation followed by hematopoietic stem cell transplantation (HSCT) as the only curative treatment. The commonly used conditioning regimens indiscriminately kill all highly proliferative cell types, leading to life threatening side effects, and are also potentially ineffective against quiescent AML subpopulations.

### Lymphoid Malignancies

Self-renewal capacity in most tissues is lost as cells progress through their normal stages of differentiation; for example, myeloid lineage blood cells beyond the level of hematopoietic stem cells no longer possess self-renewal capacity. A notable exception to differentiation-associated loss of self-renewal is the lymphoid system, where self-renewal capacity is preserved until the memory lymphocyte stage in order to maintain life-long immune memory (Fearon D T et al., Science. 2001; 293:248-250; Luckey C J et al., Proc Natl Acad Sci USA. 2006; 103: 3304-3309). Somatic hypermutation serves as a marker for the stage of differentiation at which B cell malignancies arise. In general, the presence of somatic hypermutation identifies a tumor as having arisen in germinal center or post-germinal center B cells, while the absence of mutation identifies pre-germinal center B cells. In contrast to myeloid malignancies but consonant with the lineage's preserved self-renewal capacity, immunoglobulin (Ig) mutation patterns suggest that B cell malignancies can arise from cells throughout the stages of B cell differentiation (Lapidot T et al, Nature. 1994; 367:645-648; Bonnet D and Dick J E, Nat Med. 1997, 3:730-737; Jones R J et al., J Natl Cancer Inst. 2004; 96:583-585).

### Multiple Myeloma

Multiple myeloma (MM) has generally been considered a disease of malignant plasma cells with many of the clinical consequences of the disease resulting from the plasma cell bulk. However, normal plasma cells are terminally differentiated and lack self-renewal capacity and it has been clear for over 30 years that only a minority of cells from mouse and human MM were clonogenic. These rare clonogenic cells have been termed “tumor stem cells” (Park C H et al., J Natl Cancer Inst. 1971; 46:411-422; Hamburger A W and Salmon S E, Science. 1977; 197:461-463). MM plasma cells arise from a small population of self-renewing cancer stem cells that resemble memory B cells. Not only do these clonotypic B cells circulate in most patients but they also are resistant to many standard anti-MM agents, and thus appear to be responsible for most disease relapses (Matsui W H et al., Blood. 2004; 103:2332-2336; Kukreja A et al., J Exp Med. 2006, 203:1859-1865, Jones R J and Armstrong S A, Biol Blood Marrow Transplant. 2008 January; 14 (Supplement 1): 12-16).

### Hodgkin's Lymphoma

Reed-Sternberg (RS) cells, the hallmark of Hodgkin's lymphoma (HL), are the only blood cells other than plasma cells to occasionally express CD138 (Carbone A et al., Blood. 1998; 92:2220-2228). It has been shown that HL cell lines include a small population of cells that lack the RS markers CD15 and CD30 present on the rest of the cells, while expressing markers consistent with a memory B cell phenotype (Newcom S R et al., Int J Cell Cloning. 1988; 6:417-431; Jones R J et al., Blood. 2006; 108. 470). This small subpopulation of phenotypic memory B cells possessed all of the clonogenic capacity within the HL cell lines. Most HL patients, including those with early stage disease, harbor circulating memory B cells with the same clonal Ig gene rearrangement as the patients' RS cells (Jones R J et al., Blood. 2006; 108:470; Jones R J and Armstrong S A, Biol Blood Marrow Transplant. 2008 January; 14 (Supplement 1): 12-16). These data suggest that these clonotypic memory B cells likely represent the HL stem cells.

### Treatment of Hematological Malignancies

Hematopoietic stem cells (HSCs) are used in bone marrow transplantation for treatment of hematological malignancies as well as nonmalignant disorders (Warner et al, Oncogene (2004) 23 (43): 7164-7177). Until researchers discovered which cellular components were responsible for the engraftment of the donor hematopoietic and immune systems in marrow-ablated patients, bone marrow (BM) had been transplanted as an unfractionated cell pool for many years (See, e.g., Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1). Preparation or conditioning of a patient for bone marrow/hematopoietic stem cell (BM/HSC) transplant is a critical element of the procedure. It serves two main purposes: (1) it provides adequate immunosuppression of the patient and clears sufficient niche space in the bone marrow for the transplanted HSC, which allows transplanted cells to engraft in the recipient; and (2) it often helps to eradicate the source of the malignancy.

Conditioning of patients has traditionally been achieved by administering maximally tolerated doses of a cocktail of chemotherapeutic agents with or without radiation. Components of the cocktail are often chosen to have non-overlapping toxicities. All preparative regimens currently in use are toxic and have severe side effects that can be life threatening. Among these side effects are mucositis, nausea and vomiting, alopecia, diarrhea, rash, peripheral neuropathies, infertility, pulmonary toxicities and hepatic toxicities. Many of these side effects are especially dangerous for older and sick patients, and often become a decisive component in deciding whether a patient will receive a transplant.

Thus, a need exists to prepare or condition patients eligible for bone marrow/hematopoietic stem cell (BM/HSC) transplant without these toxicities.

A need also exists to treat hematologic malignancies, such as AML, without these toxicities.

### SUMMARY

Provided herein are bispecific antibodies that bind to FLT3 and CD3 or antigen-binding fragments thereof. Also provided herein are pharmaceutical compositions comprising such antibodies or fragments. Also provided herein are methods for eliminating hematopoietic stem cells/hematopoietic progenitors (HSC/HP or, together, HSPC) and/or treating cancer in a subject using such antibodies or fragments.

In some aspects, the disclosure provides a bispecific humanized antibody or antigen binding fragment thereof that binds to human FLT3 and human CD3, wherein the antibody or fragment comprises:
(i) a first light chain variable region (VL1) comprising VL1 complementarity determining region (CDR) 1, VL1 CDR2 and VL1 CDR3, said VL1 CDR1, VL1 CDR2 and VL1 CDR3 being the CDRs of a light chain variable region (VL) that comprises (or substantially consists of or consists of) the amino acid sequence of SEQ ID NO:9; and/or
(ii) a first heavy chain variable region (VH1) comprising VH1 complementarity determining region (CDR) 1, VH1 CDR2 and VH1 CDR3, said VH1 CDR1, VH1 CDR2 and VH1 CDR3 being the CDRs of a heavy chain variable region (VH) that comprises (or substantially consists of or consists of) the amino acid sequence of SEQ ID NO:10; wherein the VL1 and the VH1 bind to human FLT3; and further comprising a second VL (VL2) and a second VH (VH2) that bind to human CD3.

In some of the foregoing or related aspects, the VL1 CDR1 comprises (or substantially consists of or consists of) the amino acid sequence QEISGY (SEQ ID NO:31), the VL1 CDR2 comprises (or substantially consists of or consists of) the amino acid sequence AAS (SEQ ID NO: 32), and the VL1 CDR3 comprises (or substantially consists of or consists of) the amino acid sequence LQYASYPLT (SEQ ID NO:37); and the VH1 CDR1 comprises (or substantially consists of or consists of) the amino acid sequence GFSLSRSTMG (SEQ ID NO:38), the VH1 CDR2 comprises (or substantially consists of or consists of) the amino acid sequence IKWNDSK (SEQ ID NO: 39), and the VH1 CDR3 comprises (or substantially consists of or consists of) the amino acid sequence ARIVYYSTYVGYFDV (SEQ ID NO:36). In some of these embodiments, the CDRs are as determined by Kabat.

In some of the foregoing or related aspects, the VL1 CDR1 comprises (or substantially consists of or consists of) the amino acid sequence RASQEISGYLS (SEQ ID NO:71), the VL1 CDR2 comprises (or substantially consists of or consists of) the amino acid sequence AASTLHS (SEQ ID NO:72), and the VL1 CDR3 comprises (or substantially consists of or consists of) the amino acid sequence LQYASYPLT (SEQ ID NO:37); and the VH1 CDR1 comprises (or substantially consists of or consists of) the amino acid sequence GFSLSRSTMGVG (SEQ ID NO: 73), the VH1 CDR2 comprises (or substantially consists of or consists of) the amino acid sequence HIKWNDSKYYNPALKS (SEQ ID NO:74), and the VH1 CDR3 comprises (or substantially consists of or consists of) the amino acid sequence IVYYSTYVGYFDV (SEQ ID NO: 75). In some of these embodiments, the CDRs are as defined by Martin (Enhanced Chothia) Numbering Scheme, as described in http://bioinf.org.uk/abs/info.html#cdrid, which is incorporated herein by reference in its entirety. In some of these embodiments, the CDRs are as defined by “How to identify the CDRs by looking at a sequence” section in http://bioinf.org.uk/abs/info.html#cdrid, which is incorporated herein by reference in its entirety.

In some of the foregoing or related aspects,
(iii) the VL2 comprises VL2 CDR1, VL2 CDR2 and VL2 CDR3, said VL2 CDR1, VL2 CDR2 and VL2 CDR3 being the CDRs of a VL that comprises (or substantially consists of or consists of) an amino acid sequence selected from the group consisting of: SEQ ID NO:4, SEQ ID NO:6, SEQ ID NO: 14, SEQ ID NO:16, SEQ ID NO:24, and SEQ ID NO:29; and/or
(iv) the VH2 comprises VH2 CDR1, VH2 CDR2 and VH2 CDR3, said VH2 CDR1, VH2 CDR2 and VH2 CDR3 being the CDRs of a VH that comprises (or substantially consists of or consists of) an amino acid sequence selected from the group consisting of: SEQ ID NO:5, SEQ ID NO:7, SEQ ID NO:12, SEQ ID NO:18, SEQ ID NO:20 and SEQ ID NO:22; wherein the VL2 and the VH2 bind to human CD3.

In some of the foregoing or related aspects,
the VL2 CDR1 comprises (or substantially consists of or consists of) the amino acid sequence of TGAVTTSNY (SEQ ID NO:40), the VL2 CDR2 comprises (or substantially consists of or consists of) the amino acid sequence of GTN (SEQ ID NO:41), and the VL2 CDR3 comprises (or substantially consists of or consists of) an amino acid sequence selected from the group consisting of: ALWYSNLWV (SEQ ID NO:42), ALWFSNHWV (SEQ ID NO:46), and ALWYSNHWV (SEQ ID NO:47); and
the VH2 CDR1 comprises (or substantially consists of or consists of) the amino acid sequence of GFTFNTYA (SEQ ID NO:43), the VH2 CDR2 comprises (or substantially consists of or consists of) the amino acid sequence of IRSKYNNYAT (SEQ ID NO:44), and the VH2 CDR3 comprises (or substantially consists of or consists of) an amino acid sequence selected from the group consisting of: VRHGNFGNSYVSWFAY (SEQ ID NO:45), VRHGNFGTSYVSWFAY (SEQ ID NO: 48), VRHGHFGTSYVSWFAY (SEQ ID NO:49), VRHGMFGTSYVSWFAY (SEQ ID NO: 50), and VRHGQFGTSYVSWFAY (SEQ ID NO:51). In some of these embodiments, the CDRs are as defined by Kabat.

In some of the foregoing or related aspects,
the VL2 CDR1 comprises (or substantially consists of or consists of) the amino acid sequence of TGAVTTSNY (SEQ ID NO:40), the VL2 CDR2 comprises (or substantially consists of or consists of) the amino acid sequence of GTN (SEQ ID NO:41), and the VL2 CDR3 comprises (or substantially consists of or consists of) an amino acid sequence selected from the group consisting of: ALWYSNLWV (SEQ ID NO:42), ALWFSNHWV (SEQ ID NO:46), and ALWYSNHWV (SEQ ID NO:47); and/or
the VH2 CDR1 comprises (or substantially consists of or consists of) the amino acid sequence of GFTFNTYA (SEQ ID NO:43), the VH2 CDR2 comprises (or substantially consists of or consists of) the amino acid sequence of IRSKYNNYAT (SEQ ID NO:44), and the VH2 CDR3 comprises (or substantially consists of or consists of) an amino acid sequence selected from the group consisting of: HGNFGNSYVSWFAY (SEQ ID NO:83), HGNFGTSYVSWFAY (SEQ ID NO: 84), HGHFGTSYVSWFAY (SEQ ID NO:85), HGMFGTSYVSWFAY (SEQ ID NO:86), and HGQFGTSYVSWFAY (SEQ ID NO:87).

In some of the foregoing or related aspects, the VL2 CDR1 comprises (or substantially consists of or consists of) the amino acid sequence of GSSTGAVTTSNYAN (SEQ ID NO:76), the VL2 CDR2 comprises (or substantially consists of or consists of) an amino acid sequence selected from the group consisting of: GTNKRSS (SEQ ID NO:77), GTNKRVS (SEQ ID NO:78), and GTNKRSS (SEQ ID NO:79) and GTNKRAS (SEQ ID NO:80); and the VL2 CDR3; and the VL2 CDR3 comprises (or substantially consists of or consists of) an amino acid sequence selected from the group consisting of: ALWYSNLWV (SEQ ID NO:42), ALWFSNHWV (SEQ ID NO: 46), and ALWYSNHWV (SEQ ID NO:47); and/or
the VH2 CDR1 comprises (or substantially consists of or consists of) the amino acid sequence of GFTENTYAMN (SEQ ID NO:81), the VH2 CDR2 comprises (or substantially consists of or consists of) the amino acid sequence of RIRSKYNNYATYYADSVKG (SEQ ID NO:82), and the VH2 CDR3 comprises (or substantially consists of or consists of) an amino acid sequence selected from the group consisting of: HGNFGNSYVSWFAY (SEQ ID NO:83), HGNFGTSYVSWFAY (SEQ ID NO:84), HGHFGTSYVSWFAY (SEQ ID NO:85), HGMFGTSYVSWFAY (SEQ ID NO: 86), and HGQFGTSYVSWFAY (SEQ ID NO:87). In some of these embodiments, the CDRs are as defined by Martin (Enhanced Chothia) Numbering Scheme, as described in http://bioinf.org.uk/abs/info.html#cdrid, which is incorporated herein by reference in its entirety. In some of these embodiments, the CDRs are as defined by “How to identify the CDRs by looking at a sequence” section in http://bioinf.org.uk/abs/info.html#cdrid, which is incorporated herein by reference in its entirety.

In some of the foregoing or related aspects, the VL2 CDR1 comprises (or substantially consists of or consists of) the amino acid sequence of GSSTGAVTTSNYAN (SEQ ID NO:76), the VL2 CDR2 comprises (or substantially consists of or consists of) an amino acid sequence selected from the group consisting of: GTNKRSS (SEQ ID NO:77), GTNKRVS (SEQ ID NO:78), and GTNKRSS (SEQ ID NO.79) and GTNKRAS (SEQ ID NO:80); and the VL2 CDR3; and the VL2 CDR3 comprises (or substantially consists of or consists of) an amino acid sequence selected from the group consisting of: ALWYSNLWV (SEQ ID NO:42), ALWFSNHWV (SEQ ID NO: 46), and ALWYSNHWV (SEQ ID NO:47); and/or
the VH2 CDR1 comprises (or substantially consists of or consists of) the amino acid sequence of GFTFNTYAMN (SEQ ID NO:81), the VH2 CDR2 comprises (or substantially consists of or consists of) the amino acid sequence of RIRSKYNNYATYYADSVKG (SEQ ID NO:82), and the VH2 CDR3 comprises (or substantially consists of or consists of) an amino acid sequence selected from the group consisting of: VRHGNFGNSYVSWFAY (SEQ ID NO:45), VRHGNFGTSYVSWFAY (SEQ ID NO:48), VRHGHFGTSYVSWFAY (SEQ ID NO:49), VRHGMFGTSYVSWFAY (SEQ ID NO:50), and VRHGQFGTSYVSWFAY (SEQ ID NO:51). In some of these embodiments, any of the CDRs are as determined by Kabat.

In some of the foregoing or related aspects,
the VL2 CDR1, VL2 CDR2 and VL2 CDR3 are the CDRs of a VL that comprises (or substantially consists of or consists of) the amino acid sequence of SEQ ID NO:4 and/or the VH2 CDR1, VH2 CDR2 and VH2 CDR3 are the CDRs of a VH that comprises (or substantially consists of or consists of) the amino acid sequence of SEQ ID NO:5. In any of the foregoing or related aspects, the VL2 CDR1 comprises (or substantially consists of or consists of) the amino acid sequence TGAVTTSNY (SEQ ID NO:40), the VL2 CDR2 comprises (or substantially consists of or consists of) the amino acid sequence GTN (SEQ ID NO:41), and the VL2 CDR3 comprises (or substantially consists of or consists of) the amino acid sequence ALWYSNLWV (SEQ ID NO:42); and/or
the VH2 CDR1 comprises (or substantially consists of or consists of) the amino acid sequence GFTFNTYA (SEQ ID NO:43), the VH2 CDR2 comprises (or substantially consists of or consists of) the amino acid sequence IRSKYNNYAT (SEQ ID NO:44), and the VH2 CDR3 comprises (or substantially consists of or consists of) the amino acid sequence VRHGNFGNSYVSWFAY (SEQ ID NO: 45). In some of these embodiments, the CDRs are as defined by Kabat. In other embodiments, the CDRs are as defined by Martin (Enhanced Chothia) Numbering Scheme or “How to identify the CDRs by looking at a sequence” section in http://bioinf.org.uk/abs/info.html#cdrid.

In some of the foregoing or related aspects,
the VL2 CDR1, VL2 CDR2 and VL2 CDR3 are the CDRs of a VL that comprises (or substantially consists of or consists of) the amino acid sequence of SEQ ID NO:4, and/or the VL2 CDR1 comprises (or substantially consists of or consists of) the amino acid sequence TGAVTTSNY (SEQ ID NO:40), the VL2 CDR2 comprises (or substantially consists of or consists of) the amino acid sequence GTN (SEQ ID NO:41), and the VL2 CDR3 comprises (or substantially consists of or consists of) the amino acid sequence ALWYSNLWV (SEQ ID NO:42); and/or
the VH2 CDR1, VH2 CDR2 and VH2 CDR3 are the CDRs of a VH that comprises (or substantially consists of or consists of) the amino acid sequence of SEQ ID NO:5, and/or the VH2 CDR1 comprises (or substantially consists of or consists of) the amino acid sequence GFTFNTYA (SEQ ID NO: 43), the VH2 CDR2 comprises (or substantially consists of or consists of) the amino acid sequence IRSKYNNYAT (SEQ ID NO:44), and the VH2 CDR3 comprises (or substantially consists of or consists of) the amino acid sequence HGNFGNSYVSWFAY (SEQ ID NO:83).

In some of the foregoing or related aspects,
the VL2 CDR1, VL2 CDR2 and VL2 CDR3 are the CDRs of a VL that comprises (or substantially consists of or consists of) the amino acid sequence of SEQ ID NO:4, and/or the VL2 CDR1 comprises (or substantially consists of or consists of) the amino acid sequence TGAVTTSNY (SEQ ID NO:40), the VL2 CDR2 comprises (or substantially consists of or consists of) the amino acid sequence GTN (SEQ ID NO:41), and the VL CDR3 comprises (or substantially consists of or consists of) the amino acid sequence ALWYSNLWV (SEQ ID NO:42); and/or
the VH2 CDR1, VH2 CDR2 and VH2 CDR3 are the CDRs of a VH that comprises (or substantially consists of or consists of) the amino acid sequence of SEQ ID NO: 12, and/or the VH2 CDR1 comprises (or substantially consists of or consists of) the amino acid sequence GFTENTYA (SEQ ID NO: 43), the VH2 CDR2 comprises (or substantially consists of or consists of) the amino acid sequence IRSKYNNYAT (SEQ ID NO:44), and the VH2 CDR3 comprises (or substantially consists of or consists of) the amino acid sequence VRHGNFGTSYVSWFAY (SEQ ID NO:48).

In some of the foregoing or related aspects,
the VL2 CDR1, VL2 CDR2 and VL2 CDR3 are the CDRs of a VL that comprises (or substantially consists of or consists of) an amino acid sequence of SEQ ID NO: 14 or SEQ ID NO: 16, and/or the VL2 CDR1 comprises (or substantially consists of or consists of) the amino acid sequence TGAVTTSNY (SEQ ID NO:40), the VL2 CDR2 comprises (or substantially consists of or consists of) the amino acid sequence GTN (SEQ ID NO:41), and the VL CDR3 comprises (or substantially consists of or consists of) the amino acid sequence ALWFSNHWV (SEQ ID NO:46); and/or
the VH2 CDR1, VH2 CDR2 and VH2 CDR3 are the CDRs of a VH that comprises (or substantially consists of or consists of) the amino acid sequence of SEQ ID NO: 12, and/or the VH2 CDR1 comprises (or substantially consists of or consists of) the amino acid sequence GFTFNTYA (SEQ ID NO: 43), the VH2 CDR2 comprises (or substantially consists of or consists of) the amino acid sequence IRSKYNNYAT (SEQ ID NO:44), and the VH2 CDR3 comprises (or substantially consists of or consists of) the amino acid sequence VRHGNFGTSYVSWFAY (SEQ ID NO:45).

In some of the foregoing or related aspects, the VL2 CDR1, VL2 CDR2 and VL2 CDR3 are the CDRs of a VL that comprises (or substantially consists of or consists of) the amino acid sequence of SEQ ID NO:14, and/or the VL2 CDR1 comprises (or substantially consists of or consists of) the amino acid sequence TGAVTTSNY (SEQ ID NO:40), the VL2 CDR2 comprises (or substantially consists of or consists of) the amino acid sequence GTN (SEQ ID NO:41), and the VL CDR3 comprises (or substantially consists of or consists of) the amino acid sequence ALWFSNHWV (SEQ ID NO:46); and/or
the VH2 CDR1, VH2 CDR2 and VH2 CDR3 are the CDRs of a VH that comprises (or substantially consists of or consists of) the amino acid sequence of SEQ ID NO.18, and/or the VH2 CDR1 comprises (or substantially consists of or consists of) the amino acid sequence GFTFNTYA (SEQ ID NO: 43), the VH2 CDR2 comprises (or substantially consists of or consists of) the amino acid sequence IRSKYNNYAT (SEQ ID NO:44), and the VH2 CDR3 comprises (or substantially consists of or consists of) the amino acid sequence VRHGHFGTSYVSWFAY (SEQ ID NO:49).

In some of the foregoing or related aspects, the VL2 CDR1, VL2 CDR2 and VL2 CDR3 are the CDRs of a VL that comprises (or substantially consists of or consists of) the amino acid sequence of SEQ ID NO: 14, and/or the VL2 CDR1 comprises (or substantially consists of or consists of) the amino acid sequence TGAVTTSNY (SEQ ID NO:40), the VL2 CDR2 comprises (or substantially consists of or consists of) the amino acid sequence GTN (SEQ ID NO:41), and the VL CDR3 comprises (or substantially consists of or consists of) the amino acid sequence ALWFSNHWV (SEQ ID NO:46); and/or
the VH2 CDR1, VH2 CDR2 and VH2 CDR3 are the CDRs of a VH that comprises (or substantially consists of or consists of) the amino acid sequence of SEQ ID NO:20, and/or the VH2 CDR1 comprises (or substantially consists of or consists of) the amino acid sequence GFTFNTYA (SEQ ID NO: 43), the VH2 CDR2 comprises (or substantially consists of or consists of) the amino acid sequence IRSKYNNYAT (SEQ ID NO:44), and the VH2 CDR3 comprises (or substantially consists of or consists of) the amino acid sequence VRHGMFGTSYVSWFAY (SEQ ID NO:50).

In some of the foregoing or related aspects,
the VL2 CDR1, VL2 CDR2 and VL2 CDR3 are the CDRs of a VL that comprises (or substantially consists of or consists of) the amino acid sequence of SEQ ID NO:14, and/or the VL2 CDR1 comprises (or substantially consists of or consists of) the amino acid sequence TGAVTTSNY (SEQ ID NO:40), the VL2 CDR2 comprises (or substantially consists of or consists of) the amino acid sequence GTN (SEQ ID NO:41), and the VL CDR3 comprises (or substantially consists of or consists of) the amino acid sequence ALWFSNHWV (SEQ ID NO.46), and/or
the VH2 CDR1, VH2 CDR2 and VH2 CDR3 are the CDRs of a VH that comprises (or substantially consists of or consists of) the amino acid sequence of SEQ ID NO:22, and/or the VH2 CDR1 comprises (or substantially consists of or consists of) the amino acid sequence GFTFNTYA (SEQ ID NO: 43), the VH2 CDR2 comprises (or substantially consists of or consists of) the amino acid sequence IRSKYNNYAT (SEQ ID NO:44), and the VH2 CDR3 comprises (or substantially consists of or consists of) the amino acid sequence VRHGQFGTSYVSWFAY (SEQ ID NO:51).

In some of the foregoing or related aspects, the VL2 CDR1, VL2 CDR2 and VL2 CDR3 are the CDRs of a VL that comprises (or substantially consists of or consists of) the amino acid sequence of SEQ ID NO:24 or SEQ ID NO:29, and/or the VL2 CDR1 comprises (or substantially consists of or consists of) the amino acid sequence TGAVTTSNY (SEQ ID NO:40), the VL2 CDR2 comprises (or substantially consists of or consists of) the amino acid sequence GTN (SEQ ID NO: 41), and the VL CDR3 comprises (or substantially consists of or consists of) the amino acid sequence ALWYSNHWV (SEQ ID NO:47); and/or
the VH2 CDR1, VH2 CDR2 and VH2 CDR3 are the CDRs of a VH that comprises (or substantially consists of or consists of) the amino acid sequence of SEQ ID NO:5, and/or the VH2 CDR1 comprises (or substantially consists of or consists of) the amino acid sequence GFTENTYA (SEQ ID NO: 43), the VH2 CDR2 comprises (or substantially consists of or consists of) the amino acid sequence IRSKYNNYAT (SEQ ID NO:44), and the VH2 CDR3 comprises (or substantially consists of or consists of) the amino acid sequence VRHGNFGNSYVSWFAY (SEQ ID NO:45).

In some aspects, the disclosure provides a bispecific humanized antibody or antigen binding fragment thereof that binds to human FLT3 and human CD3, wherein the antibody or fragment comprises:
a VL2 comprising VL2 CDR1, VL2 CDR2 and VL2 CDR3, said VL2 CDR1, VL2 CDR2 and VL2 CDR3 being the CDRs of a VL that comprises (or substantially consists of or consists of) an amino acid sequence selected from the group consisting of: SEQ ID NO:14, SEQ ID NO: 16, SEQ ID NO: 24, and SEQ ID NO:29; and/or a VH2 comprising VH2 CDR1, VH2 CDR2 and VH2 CDR3, said VH2 CDR1, VH2 CDR2 and VH2 CDR3 being the CDRs of a VH that comprises (or substantially consists of or consists of) an amino acid sequence selected from the group consisting of: SEQ ID NO:12, SEQ ID NO:18, SEQ ID NO.20, and SEQ ID NO:22; wherein the VL2 and the VH2 bind to human CD3; and further comprising a VL1 and a VH1 that bind to human FLT3.

In some of the foregoing or related aspects, the VL2 CDR1 comprises (or substantially consists of or consists of) the amino acid sequence TGAVTTSNY (SEQ ID NO:40), the VL2 CDR2 comprises (or substantially consists of or consists of) the amino acid sequence GTN (SEQ ID NO: 41), and the VL2 CDR3 comprises (or substantially consists of or consists of) the amino acid sequence ALWFSNHWV (SEQ ID NO:46) or ALWYSNHWV (SEQ ID NO:47); and/or the VH2 CDR1 comprises the amino acid sequence GFTFNTYA (SEQ ID NO:43), the VH2 CDR2 comprises (or substantially consists of or consists of) the amino acid sequence IRSKYNNYAT (SEQ ID NO:44), and the VH2 CDR3 comprises (or substantially consists of or consists of) an amino acid sequence selected from the group consisting of: VRHGNFGTSYVSFAY (SEQ ID NO: 48), VRHGHFGTSYVSFAY (SEQ ID NO:49), VRHGMFGTSYVSFAY (SEQ ID NO:50), and VRHGQFGTSYVSFAY (SEQ ID NO:51).

In some of the foregoing or related aspects, the VL1 comprises (or substantially consists of or consists of) the amino acid sequence of SEQ ID NO:9.

In some of the foregoing or related aspects, the VH1 comprises (or substantially consists of or consists of) the amino acid sequence of SEQ ID NO: 10.

In some of the foregoing or related aspects, the bispecific humanized anti-FLT3/CD3 antibodies and fragments described herein comprise a single chain variable fragment (scFv), wherein the scFv comprises the VL1 and the VH1, such as any VL1 and VH1 described herein. In some embodiments, scFv comprises (or substantially consists of or consists of) the amino acid sequence of SEQ ID NO:52.

In some of the foregoing or related aspects, the bispecific humanized anti-FLT3/CD3 antibodies described herein comprise a heavy chain (HC) and a light chain (LC), wherein the HC comprises the VL1, the VH1, and the VH2. In some embodiments, the VL1 is joined to the VH1 by a first linker, and the VH1 is joined to the VH2 by a second linker. In some embodiments, the C-terminus of the VL1 is joined to the N-terminus of the VH1 by a first linker, and the C-terminus of the VH1 is joined to the N-terminus of the VH2 by a second linker. In some embodiments, the first linker and the second linker have the formula (Gly3-4-Ser)1-4. In some embodiments, the first linker has the formula (Gly4-Ser)4. In some embodiments, the second linker has the formula (Gly4-Ser)3.

In some of the foregoing or related aspects, the bispecific humanized anti-FLT3/CD3 antibodies described herein comprise an HC that comprises (or substantially consists of or consists of) an amino acid sequence selected from the group consisting of: SEQ ID NO:53, SEQ ID NO:54, SEQ ID NO:55, SEQ ID NO:56 and SEQ ID NO:57.

In some embodiments, the HC comprises (or substantially consists of or consists of) the amino acid sequence of SEQ ID NO:53.

In some embodiments, the HC comprises (or substantially consists of or consists of) the amino acid sequence of SEQ ID NO:54.

In some embodiments, the HC comprises (or substantially consists of or consists of) the amino acid sequence of SEQ ID NO:55.

In some embodiments, the HC comprises (or substantially consists of or consists of) the amino acid sequence of SEQ ID NO:56.

In some embodiments, the HC comprises (or substantially consists of or consists of) the amino acid sequence of SEQ ID NO:57.

In some of the foregoing or related aspects, the bispecific humanized anti-FLT3/CD3 antibodies described herein comprise an LC comprising the VH2.

In some of the foregoing or related aspects, the LC comprises (or substantially consists of or consists of) an amino acid sequence selected from the group consisting of: SEQ ID NO:4, SEQ ID NO: 14, SEQ ID NO:16, SEQ ID NO:24 and SEQ ID NO:29.

In some embodiments, the LC comprises (or substantially consists of or consists of) the amino acid sequence of SEQ ID NO:4.

In some embodiments, the LC comprises (or substantially consists of or consists of) the amino acid sequence of SEQ ID NO: 14.

In some embodiments, the LC comprises (or substantially consists of or consists of) the amino acid sequence of SEQ ID NO:16.

In some embodiments, the LC comprises (or substantially consists of or consists of) the amino acid sequence of SEQ ID NO:24.

In some embodiments, the LC comprises (or substantially consists of or consists of) the amino acid sequence of SEQ ID NO:29.

In some aspects, the disclosure provides a bispecific humanized antibody or antigen binding fragment thereof that binds to human FLT3 and human CD3, wherein the antibody or fragment comprises:
(i) a first light chain variable region (VL1), wherein the VL1 comprises (or substantially consists of or consists of) the amino acid sequence of SEQ ID NO:1; and/or
(ii) a first heavy chain variable region (VH1), wherein the VH1 comprises (or substantially consists of or consists of) amino acid sequence of SEQ ID NO:2; wherein the VL1 and the VH1 bind to human FLT3; and further comprising a second light chain variable region (VL2) and a second heavy chain variable region (VH2) that bind to human CD3.

In some of the foregoing or related aspects, the VL1 comprises (or substantially consists of or consists of) the amino acid sequence of SEQ ID NO:1, and the VH1 comprises (or substantially consists of or consists of) the amino acid sequence of SEQ ID NO:2.

In some of the foregoing or related aspects, the bispecific humanized anti-FLT3/CD3 antibodies and fragments described herein comprise a single chain variable fragment (scFv), wherein the scFv comprises the VL1 and the VH1, and wherein the scFv comprises (or substantially consists of or consists of) the amino acid sequence of SEQ ID NO:3.

In some of the foregoing or related aspects,
(iii) the VL2 comprises the (or substantially consists of or consists of) amino acid sequence of SEQ ID NO:4 or SEQ ID NO:6, and/or
(iv) the VH2 comprises the (or substantially consists of or consists of) amino acid sequence of SEQ ID NO:5 or SEQ ID NO:7.

In some of the foregoing or related aspects, the VL2 comprises (or substantially consists of or consists of) the amino acid sequence of SEQ ID NO:4, and the VH2 comprises (or substantially consists of or consists of) the amino acid sequence of SEQ ID NO:5.

In some of the foregoing or related aspects, the VL2 comprises (or substantially consists of or consists of) the amino acid sequence of SEQ ID NO.6, and the VH2 comprises (or substantially consists of or consists of) the amino acid sequence of SEQ ID NO:7.

In some aspects, the disclosure provides a bispecific humanized antibody or antigen binding fragment thereof that binds to human FLT3 and human CD3, wherein the antibody or fragment comprises: a VL2 that comprises (or substantially consists of or consists of) the amino acid sequence of SEQ ID NO:4 or SEQ ID NO:6; and/or a VH2 that comprises (or substantially consists of or consists of) the amino acid sequence of SEQ ID NO:5 or SEQ ID NO:7; wherein the VL2 and the VH2 bind to human CD3; and further comprising a VL1 and a VH1 that bind to human FLT3. In some embodiments, the VL2 comprises (or substantially consists of or consists of) the amino acid sequence of SEQ ID NO:4 and the VH2 (or substantially consists of or consists of) the amino acid sequence of SEQ ID NO:5. In some embodiments, the VL2 comprises (or substantially consists of or consists of) the amino acid sequence of SEQ ID NO:6 and the VH2 (or substantially consists of or consists of) the amino acid sequence of SEQ ID NO:7.

In some of the foregoing or related aspects, the antibody comprises a heavy chain (HC) and/or a light chain (LC). In some embodiments, the LC comprises a constant domain. In some embodiments, the constant domain comprises (or substantially consists of or consists of) the amino acid sequence of SEQ ID NO:58.

In some of the foregoing or related aspects, the HC comprises an Fc region. In some embodiments, the Fc region is an IgG. In some embodiments, the IgG is a human IgG1, a human IgG2, a human IgG3, or a human IgG4. In some embodiments, the IgG is IgG1.

In some of the foregoing or related aspects, the Fc region comprises (or substantially consists of or consists of) the amino acid sequence of SEQ ID NO:59. In some of the foregoing or related aspects, the Fc region comprises (or substantially consists of or consists of) the amino acid sequence of SEQ ID NO:27.

In some of the foregoing or related aspects, the antibody comprises an LC comprising (or substantially consisting of or consisting of) the amino acid sequence of SEQ ID NO:8, and/or an HC comprising (or substantially consisting of or consisting of) the amino acid sequence of SEQ ID NO: 11.

In some of the foregoing or related aspects, the antibody comprises an LC comprising (or substantially consisting of or consisting of) the amino acid sequence of SEQ ID NO:8, and/or an HC comprising (or substantially consisting of or consisting of) the amino acid sequence of SEQ ID NO: 13.

In some of the foregoing or related aspects, the antibody comprises an LC comprising (or substantially consisting of or consisting of) the amino acid sequence of SEQ ID NO: 15, and/or an HC comprising (or substantially consisting of or consisting of) the amino acid sequence of SEQ ID NO: 13.

In some of the foregoing or related aspects, the antibody comprises an LC comprising (or substantially consisting of or consisting of) the amino acid sequence of SEQ ID NO:17, and/or an HC comprising (or substantially consisting of or consisting of) the amino acid sequence of SEQ ID NO: 13.

In some of the foregoing or related aspects, the antibody comprises an LC comprising (or substantially consisting of or consisting of) the amino acid sequence of SEQ ID NO: 17, and/or an HC comprising (or substantially consisting of or consisting of) the amino acid sequence of SEQ ID NO: 19.

In some of the foregoing or related aspects, the antibody comprises an LC comprising (or substantially consisting of or consisting of) the amino acid sequence of SEQ ID NO:17, and/or an HC comprising (or substantially consisting of or consisting of) the amino acid sequence of SEQ ID NO: 21.

In some of the foregoing or related aspects, the antibody comprises an LC comprising (or substantially consisting of or consisting of) the amino acid sequence of SEQ ID NO:17, and/or an HC comprising (or substantially consisting of or consisting of) the amino acid sequence of SEQ ID NO: 23.

In some of the foregoing or related aspects, the antibody comprises an LC comprising (or substantially consisting of or consisting of) the amino acid sequence of SEQ ID NO:25, and/or an HC comprising (or substantially consisting of or consisting of) the amino acid sequence of SEQ ID NO: 28.

In some of the foregoing or related aspects, the antibody comprises an LC comprising (or substantially consisting of or consisting of) the amino acid sequence of SEQ ID NO:30, and/or an HC comprising (or substantially consisting of or consisting of) the amino acid sequence of SEQ ID NO: 28.

In some of the foregoing or related aspects, the bispecific humanized anti-FLT3/CD3 antibody or fragment is a monoclonal antibody. In some of the foregoing or related aspects, the antibody or fragment is purified.

In some of the foregoing or related aspects, the antibody or fragment has half-life of 1 day to 14 days in a human. In some of the foregoing or related aspects, the antibody or fragment has half-life of 4 days to 7 days in a human.

In some aspects, provided is a pharmaceutical composition that comprises a therapeutically effective amount of the antibody or fragment of any one of the foregoing or related aspects and a pharmaceutically acceptable excipient. In some of the foregoing or related aspects, the pharmaceutical composition may further comprise an anti-tumor agent.

In some aspects, the disclosure provides a method of treating a hematologic cancer in a subject in need thereof, wherein the method comprises administering to the subject (e.g., a therapeutically effective amount of): (i) an antibody or fragment of any one of the foregoing or related aspects, or (ii) the pharmaceutical composition of any of the foregoing or related aspects.

In some aspects, the disclosure provides a method for preparing or conditioning a subject in need thereof for hematopoietic cell transplantation, wherein the method comprises administering to the subject (e.g. a therapeutically effective amount of): (i) an antibody or fragment of any one of the foregoing or related aspects (ii) the pharmaceutical composition of any one of the foregoing or related aspects. In some embodiments, the administering occurs prior to the hematopoietic cell transplantation. In some embodiments, the subject in need thereof has a hematologic cancer.

In some embodiments of the methods, administering of a therapeutically effective amount of antibody or fragment reduces the cell population expressing one or more of CD34, FLT3, CD33, CD11b, CD16, CD15, and CD66b by at least 90%. In some embodiments, administering of a therapeutically effective amount of antibody or fragment reduces the cell population expressing one or more of CD34, FLT3 by at least 90%.

In some embodiments of the methods, the method further comprises performing hematopoietic cell transplantation to the subject after the administering. In some embodiments, the hematopoietic cell transplantation comprises transplantation to the subject of hematopoietic stem cells and/or hematopoietic progenitor cells. In some embodiments, the performing of the hematopoietic cell transplantation occurs 5 days to 5 weeks after the administering. In some embodiments, the performing of the hematopoietic cell transplantation occurs about 2 to 3 weeks after the administering.

In some embodiments of the methods, the hematologic cancer is acute myeloid leukemia (AML), acute lymphoblastic leukemia (ALL), chronic myeloid leukemia (CML), chronic lymphocytic leukemia (CLL), peripheral T cell lymphoma, follicular lymphoma, diffuse large B cell lymphoma, Hodgkin lymphoma, non-Hodgkin lymphoma, neuroblastoma, a non-malignant inherited or acquired marrow disorder, multiple myeloma, a dendritic cell neoplasm, or blastic plasmacytoid dendritic cell neoplasm (BPDCN). In some embodiments, the hematologic cancer is AML. In some embodiments, the hematologic cancer is a dendritic cell neoplasm. In some embodiments, the hematologic cancer is blastic plasmacytoid dendritic cell neoplasm (BPDCN). In some embodiments the hematologic cancer is a non-malignant inherited or acquired marrow disorder, and wherein the non-malignant inherited or acquired marrow disorder is selected from sickle anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, or congenital thrombocytopenia.

In some embodiments of the methods, the amount of the antibody or fragment administered to a subject or therapeutically effective amount is from about 0.01 mg/kg to about 2 mg/kg. In some embodiments, the amount of the antibody or fragment administered to a subject or therapeutically effective amount is from about 0.1 mg/kg to about 0.3 mg/kg.

In some embodiments of the methods, the administering is once a single dose.

In some embodiments of the methods, the administering is every 1-14 days for about 1 to 4 weeks. In some embodiments of the methods, the administering is every 3-7 days for 2 to 3 weeks.

In some embodiments of the methods, the administering is intravenous administration (e.g., by infusion into the subject).

In some embodiments of the pharmaceutical compositions, the composition further comprises a checkpoint inhibitor.

In some embodiments of the methods, the method further comprises administration of a checkpoint inhibitor.

In some of the foregoing or related aspects, the checkpoint inhibitor is an anti-PD1 antagonist, an anti-PD-L1 antagonist and/or an anti-CTLA4 antagonist (e.g., an antagonistic antibody). In some of the foregoing or related aspects, the checkpoint inhibitor is an anti-PD1 antibody.

In some of the foregoing or related aspects, the administering of the antibody or fragment is concomitant with administration of the checkpoint inhibitor. In some of the foregoing or related aspects, the administering of the antibody or fragment is prior to administration of the checkpoint inhibitor. In some of the foregoing or related aspects, the administering of the antibody or fragment is after administration of the checkpoint inhibitor.

In some of the foregoing or related aspects, the subject is a human.

### Definitions

As used herein, the term “about,” when used to modify a numeric value, indicate that deviations of up to 10% above and below the numeric value remain within the intended meaning of the recited value.

As used herein, the term “VL” refers to the light chain variable region of an antibody.

As used herein, the term “VH” refers to the heavy chain variable region of an antibody.

As used herein, the term “percent (%) amino acid sequence identity” or “percent sequence identity” with respect to a reference polypeptide sequence is defined as the percentage of amino acid residues in a candidate sequence that are identical with the amino acid residues in the reference polypeptide sequence. Percent sequence identity is determined after aligning the sequences and introducing gaps, if necessary, to achieve the maximum percent sequence identity. Alignment for purposes of determining percent amino acid sequence identity can be achieved in various ways that are known in the art. Example alignment tools include but are not limited to BLASTp, BLAST-2, ALIGN (e.g., ALIGN-2) or Megalign (DNASTAR) software.

### BRIEF DESCRIPTION OF THE DRAWINGS

FIGS. 1A-1C show binding affinities of 118BA (FIG. 1A), 1B11E7 (FIG. 1B) and 281A (FIG. 1C) to FLT3 expressing REH cell line.

FIGS. 2A-2C show binding affinities of 118BA, 1B11E7 and 281A to FLT3 expressing REH cell line with and without pre-treatment of 10 nM FLT3L.

FIGS. 3A-3F show binding affinities of humanized variants 1 and 5 of 118BA, 7 and 10 of 1B11E7 and 1 and 5 of 281A to FLT3 expressing REH cell line, respectively.

FIGS. 4A-4C show binding profiles of 281A variant 1, 1B11E7 variant 7 and 118BA variant 1 in HEK293T cells transiently expressing rhesus FLT3 (FIG. 4A), human FLT3 (FIG. 4B) and mock (FIG. 4C) transfected cells.

FIGS. 5A and 5B show binding of anti-FLT3 humanized variants (281A variant 1, 1B11E7 variant 7 and 118BA variant 1) to FLT3 expressing CD34⁺ human and rhesus bone marrow cells and CD34 control bone marrow cells.

FIG. 6 shows binding of chimeric monoclonal anti-CD3 IgG SP34 clone to primary human cord blood T cells.

FIG. 7 shows binding of fully humanized variants 2 and 6 of monoclonal anti-CD3 IgG SP34 clone to Jurkat cells.

FIG. 8 shows binding of fully humanized variants 2 and 6 of monoclonal anti-CD3 IgG SP34 clone to around 58% of rhesus mononuclear cells in peripheral blood.

FIGS. 9A-9D show the structures of 281A #1, 1B11E7 #2, 118BA #3 and 118BA #4 bispecific antibodies, respectively, indicating FLT3 binding and CD3 binding domains.

FIGS. 10A-10D show binding affinities of 281A #1, 1B11E7 #2, 118BA #3 and 118BA #4 to FLT3 expressing REH cells, respectively.

FIGS. 11A-11D show binding affinities of 281A #1, 1B11E7 #2, 118BA #3 and 118BA #4 to FLT3 expressing Jurkat cell line, respectively.

FIGS. 12A-12D show % total apoptotic and dead target REH cells as a function of concentration of bispecific antibodies 281A #1, 1B11E7 #2, 118BA #3 and 118BA #4, respectively. The EC50 of cytotoxicity against REH cells is also indicated for each antibody.

FIG. 12E show representative flow plots of CellTrace Violet REH cells gated for Annexin V+ 7AAD+ dead cells and Annexin V+ 7AAD− apoptotic cells under various antibody concentrations in the presence and absence of effector T cells.

FIG. 12F shows % activated FSC-A high T cells as a function of concentration of bispecific antibody 118BA #3 in the presence and absence of REH target cells.

FIG. 12G shows representative flow plots of CellTrace Violet⁺ T cells gated for FSC-A high activated T cells under various antibody concentrations in the presence and absence of target REH cells.

FIG. 13 shows binding affinities of 118BA #3 to FLT3 expressing cell lines MOLM-13, OCI-AML, HL-60, NOMO-1, THP-1, MV4-11 and REH. Cells were categorized into high (EC50<1 nM), medium (1<EC50<4 nM) and low (EC50>4 nM) FLT3 expression levels.

FIG. 14 shows % total apoptotic and dead target cells (MOLM-13, OCI-AML, HL-60, NOMO-1, THP-1, MV4-11 or REH cells) as a function of concentration of bispecific antibody 118BA #3. The EC50 of cytotoxicity against each target cell line is also indicated.

FIG. 15 shows half-lives of 118BA #3 and FcRnKO variants 118BA #5 and 118BA #6 in C57BL/6 mice on a linear scale (top) and log scale (bottom).

FIGS. 16A-16D show binding affinities of 118BA #3, and its three variants 118BA #3A, 118BA #3B and 118BA #3C to FLT3 expressing REH cell line, respectively.

FIGS. 16E-16H show binding affinities of 118BA #3, and its three variants 118BA #3A, 118BA #3B and 118BA #3C to CD3 expressing human T cells, respectively.

FIGS. 17A-17D show % total apoptotic and dead target REH cells as a function of concentration of bispecific antibodies 118BA #3, 118BA #3A, 118BA #3B and 118BA #3C, respectively. The EC50 of cytotoxicity against REH cells is also indicated for each antibody if determined.

FIGS. 18A-18D show % activated FSC-A high T cells as a function of concentration of bispecific antibody 118BA #3, 118BA #3A, 118BA #3B and 118BA #3C, respectively, in the presence and absence of REH target cells.

FIGS. 19A-19D show % total apoptotic and dead target REH cells as a function of concentration of bispecific antibodies 118BA #3, 118BA #3a1, 118BA #3a2 and 118BA #3a3, respectively. The EC50 of cytotoxicity against REH cells is also indicated for each antibody if determined.

FIGS. 20A-20D show % activated FSC-A high T cells as a function of concentration of bispecific antibody 118BA #3, 118BA #3a1, 118BA #3a2 and 118BA #3a3, respectively, in the presence and absence of REH target cells.

FIGS. 21A and 21B show binding affinities of 118BA #3 and #3a1 to FLT3 expressing REH cell line, respectively. FIGS. 21C and 21D show binding affinities of 118BA #3 and #3a1 to CD3 expressing primary human T, respectively. FIG. 21E shows binding affinities of 118BA #3 and #3a1 to recombinant human CD3e coated ELISA plates.

FIGS. 22A-22E show a timeline of NOG mouse humanization and subsequent treatment schedule. A total of 3 doses were administered with 3 treatment groups and 1 control group (22A); representative images of femurs from all treatment groups at day 16, with regions of hypocellularity denoted by the arrows (22B); total numbers of mononuclear cells (MNCs) within bone marrow of femur and tibias of humanized mice. Each data point represents a single mouse and values are normalized to average counts within untreated controls (22C); total MNCs were stratified into mouse (left) and human (right) based on frequencies determined by flow cytometry. Values were normalized to average counts in untreated mice (22D); and total numbers of human CD34⁺ HSPCs from bone marrow of femur and tibias of humanized mice (22E).

FIGS. 23A-23D show a timeline of MOLM-13 engraftment and subsequent treatment schedule with 118BA #3 at doses of 0.1 and 0.01 mg/kg (23A); survival curves of all treatment groups (23B), frequency of EGFP⁺ MOLM-13 as a percentage of all mononuclear cells in peripheral blood over time for the indicated treatment group determined by flow cytometry. Each curve represents a single mouse with squares representing time of death (23C); and human T cell frequency (hCD45⁺, CD3⁺) in peripheral blood measured every two weeks up to day 30 by flow cytometry (23D).

FIGS. 24A-24D show a timeline of MV4-11 xenograft and subsequent treatment schedule with 118BA #3 at doses of 0.1 and 0.01 mg/kg (24A); survival curves of all treatment groups (24B); frequency of EGFP⁺ MOLM-13 as a percentage of all mononuclear cells in peripheral blood over time for the indicated treatment group determined by flow cytometry. Each curve represents a single mouse with squares representing time of death (24C); and human T cell frequency (hCD45⁺, CD3⁺) in peripheral blood measured every two weeks up to day 30 by flow cytometry (24D).

FIGS. 25A-25F show a timeline of NOG mouse humanization followed by EGFP-MV4-11 engraftment and treatment schedule (25A); survival curves of all treatment groups (25B); peripheral blood frequencies of EGFP-MV4-11 cells at week 4 and week 6 (25C); peripheral blood frequencies of human engraftment as a frequency of total mononuclear cells (25D); representative histograms of PD1 expression on (hCD45⁺, CD3⁺) T cells in peripheral blood 4-weeks post xenograft (25E); and peripheral blood T cell frequencies at indicated timepoint (25F).

FIGS. 26A-26C show a timeline of MV4-11 xenograft and subsequent treatment schedule with 118BA #3 variants at doses of 0.1 and 0.5 mg/kg (26A); survival curve of all treatment groups at 0.1 mg/kg (top) and 0.5 mg/kg (bottom) (26B); peripheral blood (hCD45⁺, CD3⁺) T cell frequencies at indicated timepoint for 0.1 mg/kg (top) and 0.5 mg/kg (bottom) doses (26C).

FIG. 27 shows epitope mapping on human FLT3 performed for 1B11sL3-1 antibody: difference and uptake plots comparing difference in D20 uptake between FLT3+1B11sL3-1 complex and FLT3 alone.

FIG. 28 shows epitope mapping on human FL T3 performed for 1B11sL3-1 antibody: a heat map comparing D₂O uptake between FLT3+1B11sL3-1 complex and FLT3.

FIG. 29 shows epitope mapping on human FLT3 performed for 1-18BAC1 antibody: difference and uptake plots comparing difference in D20 uptake between FLT3+1-18BAC1 complex and FLT3.

FIG. 30 shows epitope mapping on human FLT3 performed for 1-18BAC1 antibody: a heat map comparing D₂O uptake between FLT3+1-18BAC1 complex and FLT3.

### DETAILED DESCRIPTION

Provided herein are antibodies or antigen-binding fragments thereof that specifically bind to CD3 and Fms-like Tyrosine Kinase 3 (FLT3) expressing cells. Without being bound by theory, anti-CD3/anti-FLT3 antibodies described herein bind to and activate T cells and target them to FLT3 expressing target cells.

In one aspect, provided herein are bispecific antibodies or antigen binding fragments thereof specifically binding FLT3 and CD3 (such as immunoglobulins, heavy chain variable regions (VH), light chain variable regions (VL), single chain fragments (such as scFvs) and other fragments). In certain embodiments, the bispecific anti-FLT3/CD3 antibodies and antigen binding fragments thereof provided herein specifically bind human and monkey (e.g. Rhesus macaque) FLT3. In certain embodiments, the bispecific anti-FLT3/CD3 antibodies and antigen binding fragments thereof provided herein specifically bind human FLT3.

Also provided herein are pharmaceutical compositions comprising a bispecific anti-FLT3/CD3 antibody or fragment thereof described herein. In some embodiments, the pharmaceutical compositions comprise a therapeutically effective amount of the bispecific antibody or fragment (e.g., an amount used to prepare a subject for bone marrow transplantation, or for treating cancer).

Also provided herein are nucleic acids encoding the bispecific anti-FLT3/CD3 antibodies and antigen binding fragments thereof described herein. Also provided are methods of making such antibodies and fragments.

In yet another aspect, provided herein are methods of use of the bispecific anti-FLT3/CD3 antibodies and antigen binding fragments thereof described herein. In some embodiments, provided herein are methods of treatment of hematological malignancies (e.g., AML) using the bispecific antibodies and fragments described herein (e.g., by administering the antibodies or fragments to a human). In some embodiments, provided herein are methods of HSC transplant conditioning using the bispecific antibodies and fragments described herein (e.g., by administering the antibodies or fragments to a human). In some embodiments, the methods of HSC transplant conditioning can be followed by hematopoietic cell transplantation.

In any of the embodiments described herein, the bispecific anti-FLT3/CD3 antibodies and antigen binding fragments thereof can be humanized bispecific antibodies and fragments (such as antibodies and fragments comprising humanized VH and/or VL mediating binding of FLT3 and/or mediating binding of CD3). In some embodiments, the bispecific anti-FLT3/CD3 antibody or antigen binding fragment thereof comprises humanized VH mediating binding of FLT3 (such as any VH described herein). In some embodiments, the bispecific anti-FLT3/CD3 antibody or antigen binding fragment thereof comprises humanized VL mediating binding of FLT3 (such as any VL described herein). In some embodiments, the bispecific anti-FLT3/CD3 antibody or antigen binding fragment thereof comprises humanized VH and humanized VL mediating binding of FLT3 (such as any VH and VL described herein). In some embodiments, the bispecific anti-FLT3/CD3 antibody or antigen binding fragment thereof comprises humanized VH mediating binding of CD3 (such as any VH described herein). In some embodiments, the bispecific anti-FLT3/CD3 antibody or antigen binding fragment thereof comprises humanized VL mediating binding of CD3 (such as any VL described herein). In some embodiments, the bispecific anti-FLT3/CD3 antibody or antigen binding fragment thereof comprises humanized VH and humanized VL mediating binding of CD3 (such as any VH and VL described herein). In some embodiments, the bispecific anti-FLT3/CD3 antibody or antigen binding fragment thereof comprises humanized VH and humanized VL mediating binding of FLT3 and humanized VH and humanized VL mediating binding of CD3 (such as any VH and VL described herein).

### Antibodies

Provided herein are bispecific antibodies or antigen-binding fragments thereof that bind to FLT3 and CD3. In some embodiments, the bispecific antibody or fragment binds human and/or rhesus monkey FLT3. In some embodiments, the bispecific antibody or fragment binds human and/or rhesus monkey CD3. In some embodiments, the bispecific antibody or fragment binds human FLT3 and human CD3. In some embodiments, provides herein are antibodies and fragments thereof that specifically bind human FLT3 and human CD3. The antibodies and fragments described herein may display cross-reactivity with a FLT3 and/or CD3 from one or more other species (in addition to human and rhesus monkey). In some embodiments, also contemplated are antibodies and fragments thereof that specifically bind human and/or monkey (e.g., rhesus monkey) FLT3 and CD3, and do not display cross-reactivity with FLT3 from other species.

In some embodiments, the contemplated bispecific anti-FLT3/anti-CD3 antibodies and fragments comprise any CDRs described herein. In some embodiments, the contemplated bispecific anti-FLT3/anti-CD3 antibodies and fragments comprise any light chain variable region described herein and/or any heavy chain variable region described herein. In some embodiments, the contemplated bispecific anti-FLT3/anti-CD3 antibodies and fragments comprise any scFvs described herein. In some embodiments, the contemplated bispecific anti-FLT3/anti-CD3 antibodies comprise any heavy chains and/or light chains described herein. In some embodiments, the contemplated bispecific anti-FLT3/anti-CD3 antibodies comprise any constant domains described herein.

In some embodiments, the contemplated bispecific anti-FLT3/anti-CD3 antibodies and fragments comprise CDRs having at least 95% identity (e.g., amino acid identity) to any CDRs described herein. In some embodiments, the contemplated bispecific anti-FLT3/anti-CD3 antibodies and fragments comprise light chain variable region described herein and/or any heavy chain variable region having at least 95% identity (e.g., amino acid identity) to any light chain variable region described herein and/or any heavy chain variable region described herein. In some embodiments, the contemplated bispecific anti-FLT3/anti-CD3 antibodies and fragments comprise scFv having at least 95% identity (e.g., amino acid identity) to any scFvs described herein. In some embodiments, the contemplated bispecific anti-FLT3/anti-CD3 antibodies comprise heavy chain and/or light chain having 95% identity (or at least 95% identity) to any heavy chains and/or light chains described herein. In some embodiments, the contemplated bispecific anti-FLT3/anti-CD3 antibodies comprise constant domains having 95% identity (or at least 95% identity) to any constant domains described herein.

References to “fragments” throughout refer to antigen-binding fragments of the antibodies described herein.

In some embodiments, the anti-FLT3/anti-CD3 bispecific antibodies and fragments thereof described herein do not compete with FLT3 ligand for binding to FLT3.

In some embodiments, the anti-FLT3/anti-CD3 bispecific antibodies and fragments thereof described herein are bivalent on both FLT3 and CD3. In some embodiments, the anti-FLT3/anti-CD3 bispecific antibodies and fragments thereof described herein bind bivalently to FLT3 and/or CD3. In some embodiments, the anti-FLT3/anti-CD3 bispecific antibodies and fragments thereof described herein comprise 6 CDRs (3 VL and 3 VH) mediating binding to FLT3 and/or comprise 6 CDRs (3 VL and 3 VH) mediating binding to CD3, where the CDRs can be any CDRs described herein. In some embodiments, the anti-FLT3/anti-CD3 bispecific antibodies and fragments thereof described herein bind bivalently to FLT3 and CD3.

In any of the embodiments described herein, the anti-FLT3/anti-CD3 bispecific antibodies and fragments thereof can be humanized anti-FLT3/anti-CD3 bispecific antibodies and fragments thereof. The meaning of the term “humanized” is well-known in the art. A humanized antibody or fragment includes human framework regions and one or more CDRs from a non-human (e.g., a mouse, rat, or a synthetic sequence).

In some embodiments, provided herein is an anti-FLT3 1-18BA-v1 scFv (the sequence of such scFv is described herein, see the corresponding sequence in the Sequence Listing or SEQ ID NO: 3). In some embodiments, provided herein is an anti-CD3 humSP34-v6 VL (the sequence of such VL is described herein, see the corresponding sequence in the Sequence Listing or SEQ ID NO: 4). In some embodiments, provided herein is an anti-CD3 humSP34-v6 VH (the sequence of such VH is described herein, see the corresponding sequence in the Sequence Listing or SEQ ID NO.5). In some embodiments, provided herein is an anti-CD3 humSP34-v2 VL (the sequence of such VL is described herein, see SEQ ID NO:6). In some embodiments, provided herein is an anti-CD3 humSP34-v2 VH (the sequence of such VH is described herein, see the corresponding sequence in the Sequence Listing or SEQ ID NO:7). In some embodiments, provided herein are antibodies having an amino acid sequence with at least 80%, at least 85%, at least 90%, at least 95%, at least 97%, at least 98%, at least 99% amino acid identity (e.g., at least 95% identity) to any one of: anti-FLT3 1-18BA-v1 scFv, anti-CD3 humSP34-v6 VL, anti-CD3 humSP34-v6 VH, anti-CD3 humSP34-v2 VL, anti-CD3 humSP34-v2 VH. In some embodiments, the amino acid substitutions are conservative substitutions. In some embodiments, the amino acid substitutions are not in the CDR regions.

In some embodiments, provided herein is an antibody 118BA #3 WT (which is also referenced herein as #3 or #3 WT) or any antigen binding fragment thereof (the sequence of such antibody and its fragments including VH, VL and scFv are as described herein, see, e.g., the corresponding sequences in the Sequence Listing, e.g., SEQ ID NO: 8 and SEQ ID NO:11). In some embodiments, provided herein is an antibody 118BA 3A (which is also referenced herein as 3A or #3A) or any antigen binding fragment thereof (the sequence of such antibody and its fragments including VH, VL and scFv are as described herein, see, e.g., the corresponding sequences in the Sequence Listing, e.g., SEQ ID NO: 8 and SEQ ID NO:13). In some embodiments, provided herein is an antibody 118BA 3B (which is also referenced herein as 3B or #3B) or any antigen binding fragment thereof (the sequence of such antibody and its fragments including VH, VL and scFv are as described herein, see, e.g., the corresponding sequences in the Sequence Listing, e.g., SEQ ID NO: 15 and SEQ ID NO:13). In some embodiments, provided herein is an antibody 118BA 3C (which is also referenced herein as 3C or #3C) or any antigen binding fragment thereof (the sequence of such antibody and its fragments including VH, VL and scFv are as described herein, see, e.g., the corresponding sequences in the Sequence Listing, e.g., SEQ ID NO: 17 and SEQ ID NO:13). In some embodiments, provided herein is an antibody 118BA 3a2 (which is also referenced herein as 3a2 or #3a2) or any antigen binding fragment thereof (the sequence of such antibody and its fragments including VH, VL and scFv are as described herein, see, e.g., the corresponding sequences in the Sequence Listing, e.g., SEQ ID NO: 17 and SEQ ID NO: 19). In some embodiments, provided herein is an antibody 118BA 3a1 (which is also referenced herein as 3a1 or #3a1) or any antigen binding fragment thereof (the sequence of such antibody and its fragments including VH, VL and scFv are as described herein, see, e.g., the corresponding sequences in the Sequence Listing, e.g., SEQ ID NO: 17 and SEQ ID NO:21). In some embodiments, provided herein is an antibody 118BA 3a3 (which is also referenced herein as 3a3 or #3a3) or any antigen binding fragment thereof (the sequence of such antibody and its fragments including VH, VL and scFv are as described herein, see, e.g., the corresponding sequences in the Sequence Listing, e.g., SEQ ID NO: 17 and SEQ ID NO:23). In some embodiments, provided herein is an antibody 118BA #6 (which is also referenced herein as #6) or any antigen binding fragment thereof (the sequence of such antibody and its fragments including VH, VL and scFv are as described herein, see, e.g., the corresponding sequences in the Sequence Listing, e.g., SEQ ID NO: 25 and SEQ ID NO:28). In some embodiments, provided herein is an antibody 118BA #5 (which is also referenced herein as #5) or any antigen binding fragment thereof (the sequence of such antibody and its fragments including VH, VL and scFv are as described herein, see, e.g., the corresponding sequences in the Sequence Listing, e.g., SEQ ID NO: 30 and SEQ ID NO:28). Also provided herein are the 6 CDRs of any of these antibodies, including the 6 CDRs of any one of: #3WT, 3A, 3B, 3C, 3a2, 3a1, 3a3, #6 and #5 antibodies described herein. Such CDRs can be used in a different variable region framework and with different constant domains than those in any of #3WT, 3A, 3B, 3C, 3a2, 3a1, 3a3, #6 and #5 (e.g., with other variable region framework and/or constant domain(s) described herein or known in the art). In some embodiments, provided herein are antibodies having an amino acid sequence with at least 80%, at least 85%, at least 90%, at least 95%, at least 97%, at least 98%, at least 99% amino acid identity (e.g., at least 95% identity) to any one of: #3WT, 3A, 3B, 3C, 3a2, 3a1, 3a3, #6 and #5 antibodies described herein. In some embodiments, the amino acid substitutions are conservative substitutions. In some embodiments, the amino acid substitutions are not in the CDR regions.

### Complementarity-Determining Regions

In some embodiments, the CDRs of an antibody are defined according to the Chothia System. The Chothia system is based on the location of immunoglobulin structural loop regions (see, e.g., Tramontano A et al. (1990) J Mol Biol 215 (1): 175-82; Chothia C & Lesk A M, (1987), J Mol Biol 196. 901-917, U.S. Pat. No. 7,709,226; Al-Lazikani B et al., (1997) J Mol Biol 273:927-948; and Chothia C et al. (1992) J Mol Biol 227:799-817). The term “Chothia CDRs,” and like terms are recognized in the art and refer to antibody CDR sequences as determined according to the method of Chothia and Lesk, 1987, J. Mol. Biol., 196:901-917 (see also, e.g., U.S. Pat. No. 7,709,226 and Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001)). In some embodiments, the CDRs of the antibodies described herein are determined using the Chothia system.

In some embodiments, the CDRs of an antibody are defined according to the AbM System. The AbM system is based on hypervariable regions that represent a compromise between the Kabat CDRs and Chothia structural loops, and where CDRs are determined using Oxford Molecular's AbM antibody modeling software (Oxford Molecular Group, Inc.). In some embodiments, the CDRs of the antibodies described herein are determined using the AbM numbering system.

In some embodiments, the CDRs of an antibody are defined according to the IMGT system (see “IMGT®, the international ImMunoGeneTics information system® website imgt.org, founder and director: Marie-Paule Lefranc, Montpellier, France, see, e.g., Lefranc, M.-P. et al, 1999, Nucleic Acids Res., 27:209-212 and Lefranc, M.-P., 1999, The Immunologist, 7:132-136 and Lefranc, M-P. et al., 1999, Nucleic Acids Res., 27:209-212). In some embodiments, the CDRs of the antibodies described herein are determined using the IMGT system.

In some embodiments, the CDRs of an antibody are defined according to the Contact system. The Contact definition is based on an analysis of the available complex crystal structures (bioinf.org.uk/abs) (see e.g., Martin A. “Protein Sequence and Structure Analysis of Antibody Variable Domains,” in Antibody Engineering, Kontermann and Diibel, eds., Chapter 31, pp. 422-439, Springer-Verlag, Berlin (2001), and MacCallum R M et al., (1996) J Mol Biol 5:732-745). In some embodiments, the CDRs of the antibodies described herein are determined using the Contact system.

The Kabat, Chothia, AbM, IMGT and/or Contact CDR positions may vary depending on the antibody, and may be determined according to methods known in the art.

In some embodiments, the CDRs of an antibody are defined according to Martin (Enhanced Chothia) Numbering Scheme, as described in http://bioinf.org.uk/abs/info.html#cdrid, which is incorporated herein by reference in its entirety. In some embodiments, the CDRs of an antibody are defined according to “How to identify the CDRs by looking at a sequence” section in http://bioinf.org.uk/abs/info.html#cdrid, which is incorporated herein by reference in its entirety. In some embodiments, the CDR-L1 starts at approximately residue 24, and is 10 to 17 residues in length; CDR-L2 beings 16 residues after the end of CDR-L1, and is 7 residues in length; CDR-L3 begins 33 residues after the end of CDR-L2, and is 7 to 11 residues in length; CDR-H1 starts at approximately residue 26, and is 10 to 12 residues in length; CDR-H2 begins 15 residues after the end of the CDR-H1 determined by Kabat numbering, and is 16 to 19 residues in length; CDR-H3 begins 30 or 33 residues after the end of CDR-H2, and is 3 to 25 residues in length. In some embodiments, the CDRs of the antibodies described herein are determined using any of the systems described in http://bioinf.org.uk/abs/info.html#cdrid.

In some embodiments, provided herein are bispecific anti-FLT3/CD3 antibodies and fragments comprising: (i) one, two or three of the following anti-FLT3 light chain variable region CDRs (CDRs that enable binding to FLT3): a complementarity determining region 1 (CDR-L1) comprising the amino acid sequence of SEQ ID NO: 31, a complementarity determining region 2 (CDR-L2) comprising the amino acid sequence of SEQ ID NO: 32, a complementarity determining region 3 (CDR-L3) comprising the amino acid sequence of SEQ ID NO: 37, and/or (ii) one, two or three of the following anti-FLT3 heavy chain variable region CDRs (CDRs that enable binding to FLT3): a complementarity determining region 1 (CDR-H1) comprising the amino acid sequence of SEQ ID NO: 38, a complementarity determining region 2 (CDR-H2) comprising the amino acid sequence of SEQ ID NO: 39, a complementarity determining region 3 (CDR-H3) comprising the amino acid sequence of SEQ ID NO: 36. In some embodiments, a bispecific anti-FLT3/CD3 antibody or fragment comprises all 6 of these CDRs. In some embodiments, the CDRs are as defined by Kabat. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-FLT3 light chain variable region CDRs (CDRs that enable binding to FLT3): a complementarity determining region 1 (CDR-L1) comprising the amino acid sequence of SEQ ID NO: 71, a complementarity determining region 2 (CDR-L2) comprising the amino acid sequence of SEQ ID NO: 72, a complementarity determining region 3 (CDR-L3) comprising the amino acid sequence of SEQ ID NO: 37, and/or (ii) one, two or three of the following anti-FLT3 heavy chain variable region CDRs (CDRs that enable binding to FLT3): a complementarity determining region 1 (CDR-H1) comprising the amino acid sequence of SEQ ID NO: 73, a complementarity determining region 2 (CDR-H2) comprising the amino acid sequence of SEQ ID NO: 74, a complementarity determining region 3 (CDR-H3) comprising the amino acid sequence of SEQ ID NO: 75. In some embodiments, a bispecific anti-FLT3/CD3 antibody or fragment comprises all 6 of these CDRs. In some embodiments, the CDRs are as defined by Martin (Enhanced Chothia) Numbering Scheme as described in http://bioinf.org.uk/abs/info.html#cdrid, or as determined by “How to identify the CDRs by looking at a sequence” section in http://bioinf.org.uk/abs/info.html#cdrid. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO: 40, a complementarity determining region 2 (CDR-L2) comprising the amino acid sequence of SEQ ID NO: 41, a complementarity determining region 3 (CDR-L3) comprising the amino acid sequence of SEQ ID NO: 42, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID Nos: 45 or 48. In some of these embodiments, the CDR-H3 comprises an amino acid sequence of SEQ ID NO:45. In some of these embodiments, the CDR-H3 comprises an amino acid sequence of SEQ ID NO:48. In some embodiments, a bispecific anti-FLT3/CD3 antibody or fragment comprises all 6 of these CDRs. In some embodiments, the CDRs are as defined by Kabat. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO: 40, a complementarity determining region 2 (CDR-L2) comprising the amino acid sequence of SEQ ID NO: 41, a complementarity determining region 3 (CDR-L3) comprising the amino acid sequence of SEQ ID NOs: 46 or 47, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID Nos: 48-51. In some embodiments, a bispecific anti-FLT3/CD3 antibody or fragment comprises all 6 of these CDRs. In some embodiments, the CDRs are as defined by Kabat. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO: 40, a complementarity determining region 2 (CDR-L2) comprising the amino acid sequence of SEQ ID NO: 41, a complementarity determining region 3 (CDR-L3) comprising the amino acid sequence of SEQ ID NOs: 46, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID Nos: 48. In some embodiments, a bispecific anti-FLT3/CD3 antibody or fragment comprises all 6 of these CDRs. In some embodiments, the CDRs are as defined by Kabat. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO: 40, a complementarity determining region 2 (CDR-L2) comprising the amino acid sequence of SEQ ID NO: 41, a complementarity determining region 3 (CDR-L3) comprising the amino acid sequence of SEQ ID NOs: 46, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID Nos: 49. In some embodiments, a bispecific anti-FLT3/CD3 antibody or fragment comprises all 6 of these CDRs. In some embodiments, the CDRs are as defined by Kabat. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO: 40, a complementarity determining region 2 (CDR-L2) comprising the amino acid sequence of SEQ ID NO: 41, a complementarity determining region 3 (CDR-L3) comprising the amino acid sequence of SEQ ID NOs: 46, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID Nos: 50. In some embodiments, a bispecific anti-FLT3/CD3 antibody or fragment comprises all 6 of these CDRs. In some embodiments, the CDRs are as defined by Kabat. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO: 40, a complementarity determining region 2 (CDR-L2) comprising the amino acid sequence of SEQ ID NO: 41, a complementarity determining region 3 (CDR-L3) comprising the amino acid sequence of SEQ ID NOs: 46, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID Nos: 51. In some embodiments, a bispecific anti-FLT3/CD3 antibody or fragment comprises all 6 of these CDRs. In some embodiments, the CDRs are as defined by Kabat. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO: 40, a complementarity determining region 2 (CDR-L2) comprising the amino acid sequence of SEQ ID NO: 41, a complementarity determining region 3 (CDR-L3) comprising the amino acid sequence of SEQ ID NOs: 47, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID Nos: 45. In some embodiments, a bispecific anti-FLT3/CD3 antibody or fragment comprises all 6 of these CDRs. In some embodiments, the CDRs are as defined by Kabat. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO: 40, a complementarity determining region 2 (CDR-L2) comprising the amino acid sequence of SEQ ID NO: 41, a complementarity determining region 3 (CDR-L3) comprising the amino acid sequence of any one of SEQ ID NOs: 42, 46 or 47, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID NOs: 83-87. In some embodiments, a bispecific anti-FLT3/CD3 antibody or fragment comprises all 6 of these CDRs. In some embodiments, the CDRs are as defined by Kabat. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO: 76, a complementarity determining region 2 (CDR-L2) comprising the amino acid sequence of any one of SEQ ID NOs: 77-80, a complementarity determining region 3 (CDR-L3) comprising the amino acid sequence of any one of SEQ ID NOs: 42-47, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 81, a complementarity determining region 2 (CDR-H2) comprising the amino acid sequence of SEQ ID NO: 82, a complementarity determining region 3 (CDR-H3) comprising the amino acid sequence of any one of SEQ ID NOs: 83-87. In some embodiments, a bispecific anti-FLT3/CD3 antibody or fragment comprises all 6 of these CDRs. In some embodiments, the CDRs are as defined by Martin (Enhanced Chothia) Numbering Scheme as described in http://bioinf.org.uk/abs/info.html#cdrid, or as determined by “How to identify the CDRs by looking at a sequence” section in http://bioinf.org.uk/abs/info.html#cdrid. In some embodiments, the bispecific anti-FLT3/CD3 antibody or fragment is humanized.

In some embodiments, provided herein are humanized bispecific anti-FLT3/CD3 antibodies or fragments thereof, comprising (i) a VL CDR1 comprising the amino acid sequence QEISGY (SEQ ID NO:31), a CDR2 comprising the amino acid sequence AAS (SEQ ID NO:32), and a CDR3 comprising the amino acid sequence LQYASYPLT (SEQ ID NO:37); and/or (ii) a VH CDR1 comprising the amino acid sequence GFSLSRSTMG (SEQ ID NO:38), a CDR2 comprising the amino acid sequence IKWNDSK (SEQ ID NO:39), and CDR3 comprising the amino acid sequence ARIVYYSTYVGYFDV (SEQ ID NO:36).

In some embodiments, provided herein are humanized bispecific anti-FLT3/CD3 antibodies or fragments thereof, comprising (i) a VL CDR1 comprising the amino acid sequence RASQEISGYLS (SEQ ID NO:71), a CDR2 comprising the amino acid sequence AASTLHS (SEQ ID NO: 72), and a CDR3 comprising the amino acid sequence LQYASYPLT (SEQ ID NO:37); and/or (ii) a VH CDR1 comprising the amino acid sequence GFSLSRSTMGVG (SEQ ID NO:73), a CDR2 comprising the amino acid sequence HIKWNDSKYYNPALKS (SEQ ID NO:74), and CDR3 comprising the amino acid sequence IVYYSTYVGYFDV (SEQ ID NO:75).

In some embodiments, provided herein are humanized bispecific anti-FLT3/CD3 antibodies or fragments thereof, comprising (i) a VL CDR1 comprising the amino acid sequence TGAVTTSNY (SEQ ID NO:40), a CDR2 comprising the amino acid sequence GTN (SEQ ID NO: 41), and a CDR3 comprising the amino acid sequence selected from the group consisting of: ALWYSNLWV (SEQ ID NO:42), ALWFSNHWV (SEQ ID NO:46), and ALWYSNHWV (SEQ ID NO: 47); and/or (ii) a VH CDR1 comprising the amino acid sequence GFTFNTYA (SEQ ID NO.43), a CDR2 comprising the amino acid sequence IRSKYNNYAT (SEQ ID NO:44), and CDR3 comprising the amino acid sequence selected from the group consisting of: HGNFGNSYVSWFAY (SEQ ID NO:83), HGNFGTSYVSWFAY (SEQ ID NO:84), HGHFGTSYVSWFAY (SEQ ID NO:85), HGMFGTSYVSWFAY (SEQ ID NO:86), and HGQFGTSYVSWFAY (SEQ ID NO:87).

In some embodiments, provided herein are humanized bispecific anti-FLT3/CD3 antibodies or fragments thereof, comprising (i) the VL2 CDR1 comprising the amino acid sequence of GSSTGAVTTSNYAN (SEQ ID NO:76), the VL2 CDR2 comprising an amino acid sequence selected from the group consisting of: GTNKRSS (SEQ ID NO:77), GTNKRVS (SEQ ID NO: 78), and GINKRSS (SEQ ID NO:79) and GTNKRAS (SEQ ID NO:80), and the VL2 CDR3 comprising an amino acid sequence selected from the group consisting of: ALWYSNLWV (SEQ ID NO:42), ALWFSNHWV (SEQ ID NO:46), and ALWYSNHWV (SEQ ID NO:47); and/or (ii) the VH2 CDR1 comprising the amino acid sequence of GFTFNTYAMN (SEQ ID NO: 81), the VH2 CDR2 comprising the amino acid sequence of RIRSKYNNYATYYADSVKG (SEQ ID NO:82), and the VH2 CDR3 comprising an amino acid sequence selected from the group consisting of: HGNFGNSYVSWFAY (SEQ ID NO:83), HGNFGTSYVSWFAY (SEQ ID NO: 84), HGHFGTSYVSWFAY (SEQ ID NO:85), HGMFGTSYVSWFAY (SEQ ID NO:86), and HGQFGTSYVSWFAY (SEQ ID NO:87).

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO:40, a complementarity determining region 2 (CDR-L2) comprising the amino acid sequence of SEQ ID NO:41, a complementarity determining region 3 (CDR-L3) comprising the amino acid sequence of SEQ ID NOs: 42, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID NOs: 83. In some embodiments, a bispecific anti-FLT3/CD3 antibody or fragment comprises all 6 of these CDRs. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO:40, a complementarity determining region 2 (CDR-L2) comprising the amino acid sequence of SEQ ID NO:41, a complementarity determining region 3 (CDR-L3) comprising the amino acid sequence of SEQ ID NOs: 42, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID NOs: 48. In some embodiments, a bispecific anti-FLT3/CD3 antibody or fragment comprises all 6 of these CDRs. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO:40, a complementarity determining region 2 (CDR-L2) comprising the amino acid sequence of SEQ ID NO:41, a complementarity determining region 3 (CDR-L3) comprising the amino acid sequence of SEQ ID NOs: 46, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID NOs: 45. In some embodiments, a bispecific anti-FLT3/CD3 antibody or fragment comprises all 6 of these CDRs. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO:40, a complementarity determining region 2 (CDR-L2) comprising the amino acid sequence of SEQ ID NO:41, a complementarity determining region 3 (CDR-L3) comprising the amino acid sequence of SEQ ID NOs: 46, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of SEQ ID NO:49. In some embodiments, a bispecific anti-FLT3/CD3 antibody or fragment comprises all 6 of these CDRs. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO:40, a complementarity determining region 2 (CDR-L2) comprising the amino acid sequence of SEQ ID NO:41, a complementarity determining region 3 (CDR-L3) comprising the amino acid sequence of SEQ ID NOs: 46, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of any one of SEQ ID NOs: 50. In some embodiments, a bispecific anti-FLT3/CD3 antibody or fragment comprises all 6 of these CDRs. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO:40, a complementarity determining region 2 (CDR-L2) comprising the amino acid sequence of SEQ ID NO.41, a complementarity determining region 3 (CDR-L3) comprising the amino acid sequence of SEQ ID NOs: 46, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of SEQ ID NO:51. In some embodiments, a bispecific anti-FLT3/CD3 antibody or fragment comprises all 6 of these CDRs. In some embodiments, the bispecific anti-FLT3/CD3 antibody or fragment is humanized.

In some embodiments, provided herein are bispecific anti-FLT3/CD3 antibodies and fragments comprising: (i) one, two or three of the following anti-CD3 light chain variable region CDRs (CDRs that enable binding to CD3): a complementarity determining region 1 (CDR-L1) comprising the amino acid sequence of SEQ ID NO:40, a complementarity determining region 2 (CDR-L2) comprising the amino acid sequence of SEQ ID NO:41, a complementarity determining region 3 (CDR-L3) comprising the amino acid sequence of SEQ ID NOs: 47, and/or (ii) one, two or three of the following anti-CD3 heavy chain variable region CDRs (CDRs that enable binding to CD3): a complementarity determining region 1 (CDR-H1) comprising the amino acid sequence of SEQ ID NO: 43, a complementarity determining region 2 (CDR-H2) comprising the amino acid sequence of SEQ ID NO: 44, a complementarity determining region 3 (CDR-H3) comprising the amino acid sequence of SEQ ID NO:45. In some embodiments, a bispecific anti-FLT3/CD3 antibody or fragment comprises all 6 of these CDRs. In some embodiments, the bispecific anti-FLT3/CD3 antibody or fragment is humanized.

In some embodiments, provided herein are bispecific anti-FLT3/CD3 antibodies and fragments comprising the CDRs of any of the VL and VH described herein individually and in combination.

### Additional CDR Disclosures

In some embodiments, provided herein are anti-FLT3 antibodies or fragments thereof having a light chain variable region comprising a complementarity determining region 1 (CDR-L1) having the amino acid sequence of SEQ ID NO: 71. In some embodiments, provided herein are anti-FLT3 antibodies or fragments thereof having a light chain variable region comprising a complementarity determining region 2 (CDR-L2) having the amino acid sequence of SEQ ID NO: 72.

In some embodiments, provided herein are anti-FLT3 antibodies or fragments thereof having a heavy chain variable region comprising a complementarity determining region 1 (CDR-H1) having the amino acid sequence of SEQ ID NO: 73. In some embodiments, provided herein are anti-FLT3 antibodies or fragments thereof having a heavy chain variable region comprising a complementarity determining region 2 (CDR-H2) having the amino acid sequence of SEQ ID NO. 74. In some embodiments, provided herein are anti-FLT3 antibodies or fragments thereof having a heavy chain variable region comprising a complementarity determining region 3 (CDR-H3) having the amino acid sequence of SEQ ID NO: 75.

In some embodiments, provided herein are anti-CD3 antibodies or fragments thereof having a light chain variable region comprising a complementarity determining region 1 (CDR-L1) having the amino acid sequence of SEQ ID NO: 76. In some embodiments, provided herein are anti-CD3 antibodies or fragments thereof having a light chain variable region comprising a complementarity determining region 2 (CDR-L2) having the amino acid sequence selected from the group comprising: SEQ ID NOs: 77-80. In some embodiments, provided herein are anti-CD3 antibodies or fragments thereof having a light chain variable region comprising a complementarity determining region 2 (CDR-L2) having the amino acid sequence of SEQ ID NO: 77. In some embodiments, provided herein are anti-CD3 antibodies or fragments thereof having a light chain variable region comprising a complementarity determining region 2 (CDR-L2) having the amino acid sequence of SEQ ID NO: 78. In some embodiments, provided herein are anti-CD3 antibodies or fragments thereof having a light chain variable region comprising a complementarity determining region 2 (CDR-L2) having the amino acid sequence of SEQ ID NO: 79. In some embodiments, provided herein are anti-CD3 antibodies or fragments thereof having a light chain variable region comprising a complementarity determining region 2 (CDR-L2) having the amino acid sequence of SEQ ID NO: 80.

In some embodiments, provided herein are anti-CD3 antibodies or fragments thereof having a heavy chain variable region comprising a complementarity determining region 1 (CDR-H1) having the amino acid sequence of SEQ ID NO: 81. In some embodiments, provided herein are anti-CD3 antibodies or fragments thereof having a heavy chain variable region comprising a complementarity determining region 2 (CDR-H2) having the amino acid sequence of SEQ ID NO: 82. In some embodiments, provided herein are anti-CD3 antibodies or fragments thereof having a heavy chain variable region comprising a complementarity determining region 3 (CDR-H3) having the amino acid sequence selected from the group comprising SEQ ID NOs: 83-87. In some embodiments, provided herein are anti-CD3 antibodies or fragments thereof having a heavy chain variable region comprising a complementarity determining region 3 (CDR-H3) having the amino acid sequence of SEQ ID NO: 83. In some embodiments, provided herein are anti-CD3 antibodies or fragments thereof having a heavy chain variable region comprising a complementarity determining region 3 (CDR-H3) having the amino acid sequence of SEQ ID NO: 84. In some embodiments, provided herein are anti-CD3 antibodies or fragments thereof having a heavy chain variable region comprising a complementarity determining region 3 (CDR-H3) having the amino acid sequence of SEQ ID NO: 85. In some embodiments, provided herein are anti-CD3 antibodies or fragments thereof having a heavy chain variable region comprising a complementarity determining region 3 (CDR-H3) having the amino acid sequence of SEQ ID NO: 86. In some embodiments, provided herein are anti-CD3 antibodies or fragments thereof having a heavy chain variable region comprising a complementarity determining region 3 (CDR-H3) having the amino acid sequence of SEQ ID NO: 87.

In some embodiments, provided herein are humanized anti-FLT3 antibodies or fragments thereof having a light chain variable region comprising a complementarity determining region 1 (CDR-L1) having the amino acid sequence of SEQ ID NO: 31. In some embodiments, provided herein are humanized anti-FLT3 antibodies or fragments thereof having a light chain variable region comprising a complementarity determining region 2 (CDR-L2) having the amino acid sequence of SEQ ID NO: 32. In some embodiments, provided herein are humanized anti-FLT3 antibodies or fragments thereof having a light chain variable region comprising a complementarity determining region 3 (CDR-L3) having the amino acid sequence of SEQ ID NO: 33. In some embodiments, provided herein are anti-FLT3 antibodies or fragments thereof having a light chain variable region comprising a complementarity determining region 3 (CDR-L3) having the amino acid sequence of SEQ ID NO: 37.

In some embodiments, provided herein are humanized anti-FLT3 antibodies or fragments thereof having a heavy chain variable region comprising a complementarity determining region 1 (CDR-H1) having the amino acid sequence of SEQ ID NO: 34. In some embodiments, provided herein are humanized anti-FLT3 antibodies or fragments thereof having a heavy chain variable region comprising a complementarity determining region 1 (CDR-H1) having the amino acid sequence of SEQ ID NO: 38. In some embodiments, provided herein are humanized anti-FLT3 antibodies or fragments thereof having a heavy chain variable region comprising a complementarity determining region 2 (CDR-H2) having the amino acid sequence of SEQ ID NO: 35. In some embodiments, provided herein are anti-FLT3 antibodies or fragments thereof having a heavy chain variable region comprising a complementarity determining region 2 (CDR-H2) having the amino acid sequence of SEQ ID NO: 39. In some embodiments, provided herein are humanized anti-FLT3 antibodies or fragments thereof having a heavy chain variable region comprising a complementarity determining region 3 (CDR-H3) having the amino acid sequence of SEQ ID NO: 36.

In some embodiments, provided herein are humanized anti-CD3 antibodies or fragments thereof having a light chain variable region comprising a complementarity determining region 1 (CDR-L1) having the amino acid sequence of SEQ ID NO: 40. In some embodiments, provided herein are humanized anti-CD3 antibodies or fragments thereof having a light chain variable region comprising a complementarity determining region 2 (CDR-L2) having the amino acid sequence of SEQ ID NO: 41. In some embodiments, provided herein are humanized anti-CD3 antibodies or fragments thereof having a light chain variable region comprising a complementarity determining region 2 (CDR-L2) having the amino acid sequence of SEQ ID NO: 42. In some embodiments, provided herein are anti-CD3 antibodies or fragments thereof having a light chain variable region comprising a complementarity determining region 2 (CDR-L2) having the amino acid sequence of SEQ ID NO: 46. In some embodiments, provided herein are anti-CD3 antibodies or fragments thereof having a light chain variable region comprising a complementarity determining region 2 (CDR-L2) having the amino acid sequence of SEQ ID NO: 47.

In some embodiments, provided herein are humanized anti-CD3 antibodies or fragments thereof having a heavy chain variable region comprising a complementarity determining region 1 (CDR-H1) having the amino acid sequence of SEQ ID NO: 43. In some embodiments, provided herein are humanized anti-CD3 antibodies or fragments thereof having a heavy chain variable region comprising a complementarity determining region 2 (CDR-H2) having the amino acid sequence of SEQ ID NO: 44. In some embodiments, provided herein are humanized anti-CD3 antibodies or fragments thereof having a heavy chain variable region comprising a complementarity determining region 3 (CDR-H3) having the amino acid sequence of SEQ ID NO: 45. In some embodiments, provided herein are anti-CD3 antibodies or fragments thereof having a heavy chain variable region comprising a complementarity determining region 3 (CDR-H3) having the amino acid sequence of SEQ ID NO: 48. In some embodiments, provided herein are anti-CD3 antibodies or fragments thereof having a heavy chain variable region comprising a complementarity determining region 3 (CDR-H3) having the amino acid sequence of SEQ ID NO: 49. In some embodiments, provided herein are anti-CD3 antibodies or fragments thereof having a heavy chain variable region comprising a complementarity determining region 3 (CDR-H3) having the amino acid sequence of SEQ ID NO: 50. In some embodiments, provided herein are anti-CD3 antibodies or fragments thereof having a heavy chain variable region comprising a complementarity determining region 3 (CDR-H3) having the amino acid sequence of SEQ ID NO: 51.

In some embodiments, provided herein are anti-FLT3 antibodies or fragments thereof having a light chain variable region comprising CDR-L1, CDR-L2 and CDR-L3 having SEQ ID NOs: 31, 32, and 37, respectively. In certain embodiments, the anti-FLT3 antibodies or fragments are humanized.

In some embodiments, provided herein are anti-FLT3 antibodies or fragments thereof having a heavy chain variable region comprising CDR-H1, CDR-H2 and CDR-H3 having SEQ ID NOs: 38, 39, and 36, respectively. In certain embodiments, the anti-FLT3 antibodies or fragments are humanized.

In some embodiments, provided herein are humanized bispecific antibodies or fragments thereof, comprising a VL CDR1 comprising the amino acid sequence QEISGY (SEQ ID NO:31), a CDR2 comprising the amino acid sequence AAS (SEQ ID NO:32), and a CDR3 comprising the amino acid sequence LQYASYPLT (SEQ ID NO:37); and a VH CDR1 comprising the amino acid sequence GFSLSRSTMG (SEQ ID NO:38), a CDR2 comprising the amino acid sequence IKWNDSK (SEQ ID NO:39), and CDR3 comprising the amino acid sequence ARIVYYSTYVGYFDV (SEQ ID NO:36).

In some embodiments, provided herein are anti-FLT3 antibodies or fragments thereof having a light chain variable region comprising CDR-L1, CDR-L2 and CDR-L3 having SEQ ID NOs: 71, 72, and 37, respectively. In certain embodiments, the anti-FLT3 antibodies or fragments are humanized.

In some embodiments, provided herein are anti-FLT3 antibodies or fragments thereof having a heavy chain variable region comprising CDR-H1, CDR-H2 and CDR-H3 having SEQ ID NOs: 73, 74, and 75, respectively. In certain embodiments, the anti-FLT3 antibodies or fragments are humanized.

In some embodiments, provided herein are humanized bispecific antibodies or fragments thereof, comprising a VL CDR1 comprising the amino acid sequence RASQEISGYLS (SEQ ID NO: 71), a CDR2 comprising the amino acid sequence AASTLHS (SEQ ID NO:72), and a CDR3 comprising the amino acid sequence LQYASYPLT (SEQ ID NO:37); and a VH CDR1 comprising the amino acid sequence GFSLSRSTMGVG (SEQ ID NO: 73), a CDR2 comprising the amino acid sequence HIKWNDSKYYNPALKS (SEQ ID NO:74), and CDR3 comprising the amino acid sequence IVYYSTYVGYFDV (SEQ ID NO:75).

In some embodiments, provided herein are anti-CD3 antibodies or fragments thereof having a light chain variable region comprising CDR-L1, CDR-L2 and CDR-L3 having SEQ ID NOs: 40, 41, and 42, respectively. In some embodiments, provided herein are anti-CD3 antibodies or fragments thereof having a light chain variable region comprising CDR-L1, CDR-L2 and CDR-L3 having SEQ ID NOs: 40, 41, and 46, respectively. In some embodiments, provided herein are anti-CD3 antibodies or fragments thereof having a light chain variable region comprising CDR-L1, CDR-L2 and CDR-L3 having SEQ ID NOs: 40, 41, and 47, respectively. In certain embodiments, the anti-CD3 antibodies or fragments are humanized.

In some embodiments, provided herein are anti-CD3 antibodies or fragments thereof having a heavy chain variable region comprising CDR-H1, CDR-H2 and CDR-H3 having SEQ ID NOs: 43, 44, and 45, respectively. In some embodiments, provided herein are anti-CD3 antibodies or fragments thereof having a heavy chain variable region comprising CDR-H1, CDR-H2 and CDR-H3 having SEQ ID NOs: 43, 44, and 48, respectively. In some embodiments, provided herein are anti-CD3 antibodies or fragments thereof having a heavy chain variable region comprising CDR-H1, CDR-H2 and CDR-H3 having SEQ ID NOs: 43, 44, and 49, respectively. In some embodiments, provided herein are anti-CD3 antibodies or fragments thereof having a heavy chain variable region comprising CDR-H1, CDR-H2 and CDR-H3 having SEQ ID NOs: 43, 44, and 50, respectively. In some embodiments, provided herein are anti-CD3 antibodies or fragments thereof having a heavy chain variable region comprising CDR-H1, CDR-H2 and CDR-H3 having SEQ ID NOs: 43, 44, and 51, respectively. In certain embodiments, the anti-CD3 antibodies or fragments are humanized.

In some embodiments, provided herein are humanized bispecific antibodies or fragments thereof, comprising a VL CDR1 comprising the amino acid sequence TGAVTTSNY (SEQ ID NO: 40), a CDR2 comprising the amino acid sequence GTN (SEQ ID NO:41), and a CDR3 comprising the amino acid sequence selected from the group consisting of: ALWYSNLWV (SEQ ID NO: 42), ALWFSNHWV (SEQ ID NO:46), and ALWYSNHWV (SEQ ID NO.47), and a VH CDR1 comprising the amino acid sequence GFTFNTYA (SEQ ID NO:43), a CDR2 comprising the amino acid sequence IRSKYNNYAT (SEQ ID NO:44), and CDR3 comprising the amino acid sequence selected from the group consisting of: HGNFGNSYVSWFAY (SEQ ID NO:83), HGNFGTSYVSWFAY (SEQ ID NO:84), HGHFGTSYVSWFAY (SEQ ID NO:85), HGMFGTSYVSWFAY (SEQ ID NO:86), and HGQFGTSYVSWFAY (SEQ ID NO:87).

In some embodiments, provided herein are anti-CD3 antibodies or fragments thereof having a light chain variable region comprising CDR-L1, CDR-L2 and CDR-L3 having SEQ ID NOs: 76, 77, and 42, respectively. In some embodiments, provided herein are anti-CD3 antibodies or fragments thereof having a light chain variable region comprising CDR-L1, CDR-L2 and CDR-L3 having SEQ ID NOs: 76, 78, and 42, respectively. In some embodiments, provided herein are anti-CD3 antibodies or fragments thereof having a light chain variable region comprising CDR-L1, CDR-L2 and CDR-L3 having SEQ ID NOs: 76, 79, and 42, respectively. In some embodiments, provided herein are anti-CD3 antibodies or fragments thereof having a light chain variable region comprising CDR-L1, CDR-L2 and CDR-L3 having SEQ ID NOs: 76, 80, and 42, respectively. In some embodiments, provided herein are anti-CD3 antibodies or fragments thereof having a light chain variable region comprising CDR-L1, CDR-L2 and CDR-L3 having SEQ ID NOs: 76, 77, and 46, respectively. In some embodiments, provided herein are anti-CD3 antibodies or fragments thereof having a light chain variable region comprising CDR-L1, CDR-L2 and CDR-L3 having SEQ ID NOs: 76, 78, and 46, respectively. In some embodiments, provided herein are anti-CD3 antibodies or fragments thereof having a light chain variable region comprising CDR-L1, CDR-L2 and CDR-L3 having SEQ ID NOs: 76, 79, and 46, respectively. In some embodiments, provided herein are anti-CD3 antibodies or fragments thereof having a light chain variable region comprising CDR-L1, CDR-L2 and CDR-L3 having SEQ ID NOs: 76, 80, and 46, respectively. In some embodiments, provided herein are anti-CD3 antibodies or fragments thereof having a light chain variable region comprising CDR-L1, CDR-L2 and CDR-L3 having SEQ ID NOs: 76, 77, and 47, respectively. In some embodiments, provided herein are anti-CD3 antibodies or fragments thereof having a light chain variable region comprising CDR-L1, CDR-L2 and CDR-L3 having SEQ ID NOs: 76, 78, and 47, respectively. In some embodiments, provided herein are anti-CD3 antibodies or fragments thereof having a light chain variable region comprising CDR-L1, CDR-L2 and CDR-L3 having SEQ ID NOs: 76, 79, and 47, respectively. In some embodiments, provided herein are anti-CD3 antibodies or fragments thereof having a light chain variable region comprising CDR-L1, CDR-L2 and CDR-L3 having SEQ ID NOs: 76, 80, and 47, respectively. In certain embodiments, the anti-CD3 antibodies or fragments are humanized.

In some embodiments, provided herein are anti-CD3 antibodies or fragments thereof having a heavy chain variable region comprising CDR-H1, CDR-H2 and CDR-H3 having SEQ ID NOs: 43, 44, and 45, respectively. In some embodiments, provided herein are anti-CD3 antibodies or fragments thereof having a heavy chain variable region comprising CDR-H1, CDR-H2 and CDR-H3 having SEQ ID NOs: 43, 44, and 48, respectively. In some embodiments, provided herein are anti-CD3 antibodies or fragments thereof having a heavy chain variable region comprising CDR-H1, CDR-H2 and CDR-H3 having SEQ ID NOs: 43, 44, and 49, respectively. In some embodiments, provided herein are anti-CD3 antibodies or fragments thereof having a heavy chain variable region comprising CDR-H1, CDR-H2 and CDR-H3 having SEQ ID NOs: 43, 44, and 50, respectively. In some embodiments, provided herein are anti-CD3 antibodies or fragments thereof having a heavy chain variable region comprising CDR-H1, CDR-H2 and CDR-H3 having SEQ ID NOs: 43, 44, and 51, respectively. In certain embodiments, the anti-CD3 antibodies or fragments are humanized.

In some embodiments, provided herein are anti-CD3 antibodies or fragments thereof having a heavy chain variable region comprising CDR-H1, CDR-H2 and CDR-H3 having SEQ ID NOs: 81, 82, and 45, respectively. In some embodiments, provided herein are anti-CD3 antibodies or fragments thereof having a heavy chain variable region comprising CDR-H1, CDR-H2 and CDR-H3 having SEQ ID NOs: 81, 82, and 48, respectively. In some embodiments, provided herein are anti-CD3 antibodies or fragments thereof having a heavy chain variable region comprising CDR-H1, CDR-H2 and CDR-H3 having SEQ ID NOs: 81, 82, and 49, respectively. In some embodiments, provided herein are anti-CD3 antibodies or fragments thereof having a heavy chain variable region comprising CDR-H1, CDR-H2 and CDR-H3 having SEQ ID NOs: 81, 82, and 50, respectively. In some embodiments, provided herein are anti-CD3 antibodies or fragments thereof having a heavy chain variable region comprising CDR-H1, CDR-H2 and CDR-H3 having SEQ ID NOs: 81, 82, and 51, respectively. In certain embodiments, the anti-CD3 antibodies or fragments are humanized.

In certain embodiments, any of the antibodies or fragments contemplated herein may comprise any of the above-described CDRs. In some embodiments, any such antibodies or fragments can be humanized antibodies or fragments. In some embodiments, any such antibodies or fragments can be anti-FLT3/CD3 bispecific antibodies or fragments. In some embodiments, any such antibodies or fragments can be humanized and anti-FLT3/CD3 bispecific antibodies or fragments.

### VL and VH

In some embodiments, the bispecific humanized antibody or antigen binding fragment binds to human FLT3 and human CD3, wherein the antibody or fragment comprises:
(i) a first light chain variable region (VL1); and
(ii) a first heavy chain variable region (VH1); wherein the VL1 and the VH1 bind to human FLT3; and further comprising a second VL (VL2) and a second VH (VH2) that bind to human CD3.

In some embodiments, the humanized bispecific anti-FLT3/CD3 antibodies and fragments contemplated herein comprise any VL described herein.

In some embodiments, the humanized bispecific anti-FLT3/CD3 antibodies and fragments contemplated herein comprise any VH described herein.

In some embodiments, the humanized bispecific anti-FLT3/CD3 antibodies and fragments contemplated herein comprise an anti-FLT3 VL selected from SEQ ID NOs: 1 and 9. In some embodiments, the VL comprises SEQ ID NO: 1. In some embodiments, the VL comprises an amino acid sequence having at least 80%, at least 85%, at least 90%, at least 95%, or at least 99% sequence identity to SEQ ID NO:1 (e.g., at least 95% identity). In some embodiments, the VL comprises SEQ ID NO: 9. In some embodiments, the VL comprises an amino acid sequence having at least 80%, at least 85%, at least 90%, at least 95%, or at least 99% sequence identity to SEQ ID NO: 9 (e.g., at least 95% identity). In certain embodiments, substitutions, insertions or deletions in these sequences occur in regions outside the CDRs (i.e., in the framework regions). In certain embodiments, provided herein are antibodies or fragments thereof comprising (i) a VL1 comprising an amino acid sequence having at least 95%, at least 96%, at least 97%, at least 98%, or at least 99% identity to the amino acid sequence of SEQ ID NO: 9, with at least 95% (or at least 96%, 97%, 98%, 99% or 100%) identity in the framework regions and at least 97% (or at least 98%, 99% or 100% identity) in the CDR regions.

In some embodiments, the anti-FLT3 light chain variable region (VL) comprises one or more of the following mutations: L36Y, G41E, 144P, R46S, Q55H, R66G, S69T, Y7IF, F95L, T116A in SEQ ID NO: 1 (e.g., as in SEQ ID NO:9).

In some embodiments, the humanized bispecific anti-FLT3/CD3 antibodies and fragments contemplated herein comprise an anti-FLT3 VH selected from SEQ ID NOs: 2 and 10. In some embodiments, the VH comprises SEQ ID NO: 2. In some embodiments, the VH comprises an amino acid sequence having at least 80%, at least 85%, at least 90%, at least 95%, or at least 99% sequence identity to SEQ ID NO:2 (e.g., at least 95% identity). In some embodiments, the VH comprises SEQ ID NO: 10. In some embodiments, the VH comprises an amino acid sequence having at least 80%, at least 85%, at least 90%, at least 95%, or at least 99% sequence identity to SEQ ID NO:10 (e.g., at least 95% identity). In certain embodiments, substitutions, insertions or deletions in these sequences occur in regions outside the CDRs (i.e., in the framework regions). In certain embodiments, provided herein are a antibodies or fragments thereof comprising (i) a VH1 comprising an amino acid sequence having at least 95%, at least 96%, at least 97%, at least 98%, or at least 99% identity to the amino acid sequence of SEQ ID NO: 10, with at least 95% (or at least 96%, 97%, 98%, 99% or 100%) identity in the framework regions and at least 97% (or at least 98%, 99% or 100% identity) in the CDR regions.

In certain embodiments, provided herein are the humanized bispecific anti-FLT3/CD3 antibodies and fragments comprising: (i) a first heavy chain variable region (VH1) comprising the amino acid sequence of SEQ ID NO: 10, and/or (ii) a first light chain variable region (VL1) comprising the amino acid sequence of SEQ ID NO: 9. In some embodiments, provided herein are bispecific humanized anti-FLT3/anti-CD3 antibodies or fragments thereof comprising (i) a VH1 comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 10, and/or (ii) a VL1 comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 9. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a VH1 comprising an amino acid sequence having at least 95%, at least 96%, at least 97%, at least 98%, or at least 99% identity to the amino acid sequence of SEQ ID NO: 10, with at least 95% (or at least 96%, 97%, 98%, 99% or 100%) identity in the framework regions and at least 97% (or at least 98%, 99% or 100% identity) in the CDR regions, and/or (ii) a VL1 comprising an amino acid sequence having at least 95%, at least 96%, at least 97%, at least 98%, or at least 99% identity to the amino acid sequence of SEQ ID NO: 9, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof (e.g., scFv) comprising both the VH1 and the VL1 comprising the sequences specified in this paragraph. In some embodiments, provided herein are bispecific humanized anti-FLT3/anti-CD3 antibodies or fragments thereof comprising the amino acid sequence of SEQ ID NO:52.

In some embodiments, the anti-FLT3 heavy chain variable region (VH) comprises one or more of the following mutations: T157R, L180K, R186Y, S190A, T198S, K204N, T244L in SEQ ID NO. 2 (e.g., as in SEQ ID NO.10).

In some embodiments, the humanized bispecific anti-FLT3/CD3 antibodies and fragments contemplated herein comprise an anti-CD3 VL selected from SEQ ID NOs: 4, 6, 14, 16, 24, and 29. In some embodiments, the VL comprises an amino acid sequence having at least 80%, at least 85%, at least 90%, at least 95%, or at least 99% sequence identity to a sequence selected from SEQ ID NOs: 4, 6, 14, 16, 24, and 29 (e.g., at least 95% identity). In some embodiments, the VL comprises SEQ ID NO: 4. In some embodiments, the VL comprises SEQ ID NO: 6. In some embodiments, the VL comprises SEQ ID NO. 14. In some embodiments, the VL comprises SEQ ID NO: 16. In some embodiments, the VL comprises SEQ ID NO: 24. In some embodiments, the VL comprises SEQ ID NO: 29. In some embodiments, the VL comprises an amino acid sequence having at least 80%, at least 85%, at least 90%, at least 95%, or at least 99% sequence identity to the sequence of SEQ ID NO: 4 (e.g., at least 95% identity). In some embodiments, the VL comprises an amino acid sequence having at least 80%, at least 85%, at least 90%, at least 95%, or at least 99% sequence identity to the sequence of SEQ ID NO: 6 (e.g., at least 95% identity). In some embodiments, the VL comprises an amino acid sequence having at least 80%, at least 85%, at least 90%, at least 95%, or at least 99% sequence identity to the sequence of SEQ ID NO. 14 (e.g., at least 95% identity). In some embodiments, the VL comprises an amino acid sequence having at least 80%, at least 85%, at least 90%, at least 95%, or at least 99% sequence identity to the sequence of SEQ ID NO: 16 (e.g., at least 95% identity). In some embodiments, the VL comprises an amino acid sequence having at least 80%, at least 85%, at least 90%, at least 95%, or at least 99% sequence identity to the sequence of SEQ ID NO: 24 (e.g., at least 95% identity). In some embodiments, the VL comprises an amino acid sequence having at least 80%, at least 85%, at least 90%, at least 95%, or at least 99% sequence identity to the sequence of SEQ ID NO: 29 (e.g., at least 95% identity).

In some embodiments, the anti-CD3 light chain variable region (VL) comprises one or more (2, 3, 4, 5, 6, 7, 8, 9, 10, 11, 12, 13, 14, 15, 16, of any combination of, or all) of the following mutations: L10F, S11T, T41K, G48T, R56K, S57H, S58P, G59W, V60T, D62A, D71G, 176L, T77S, A81P, D82E, S85A, and D86E, in SEQ ID NO: 4 (e.g., as in SEQ ID NO: 6).

In some embodiments, the light chain variable region (VL) comprises one or more mutations of: S57V, Y94F, and L97H in respect to SEQ ID NO: 4 (e.g., as in SEQ ID NO: 14).

In some embodiments, the light chain variable region (VL) comprises one or more mutations of: Y94F and L97H in respect to SEQ ID NO. 4 (e.g., as in SEQ ID NO: 16).

In some embodiments, the light chain variable region (VL) comprises one or more mutations of: S67A and L97H in respect to SEQ ID NO: 4 (e.g., as in SEQ ID NO: 29).

In some embodiments, the humanized bispecific anti-FLT3/CD3 antibodies and fragments contemplated herein comprise an anti-CD3 VH selected from SEQ ID NOs: 5, 7, 12, 18, 20 and 22. In some embodiments, the VH comprises SEQ ID NO: 5. In some embodiments, the VH comprises SEQ ID NO: 7. In some embodiments, the VH comprises SEQ ID NO: 12. In some embodiments, the VH comprises SEQ ID NO: 18. In some embodiments, the VH comprises SEQ ID NO: 20. In some embodiments, the VH comprises SEQ ID NO: 22. In some embodiments, the VH comprises an amino acid sequence having at least 80%, at least 85%, at least 90%, at least 95%, or at least 99% sequence identity to the sequence of SEQ ID NO: 5 (e.g., at least 95% identity). In some embodiments, the VH comprises an amino acid sequence having at least 80%, at least 85%, at least 90%, at least 95%, or at least 99% sequence identity to the sequence of SEQ ID NO. 7 (e.g., at least 95% identity). In some embodiments, the VH comprises an amino acid sequence having at least 80%, at least 85%, at least 90%, at least 95%, or at least 99% sequence identity to the sequence of SEQ ID NO: 12 (e.g., at least 95% identity). In some embodiments, the VH comprises an amino acid sequence having at least 80%, at least 85%, at least 90%, at least 95%, or at least 99% sequence identity to the sequence of SEQ ID NO: 18 (e.g., at least 95% identity). In some embodiments, the VL comprises an amino acid sequence having at least 80%, at least 85%, at least 90%, at least 95%, or at least 99% sequence identity to the sequence of SEQ ID NO: 20 (e.g., at least 95% identity). In some embodiments, the VL comprises an amino acid sequence having at least 80%, at least 85%, at least 90%, at least 95%, or at least 99% sequence identity to the sequence of SEQ ID NO: 22 (e.g., at least 95% identity).

In some embodiments, the anti-CD3 heavy chain variable region (VH) comprises the mutation N79S in SEQ ID NO: 5 (e.g., as in SEQ ID NO: 7).

In some embodiments, the anti-CD3 heavy chain variable region (VH) comprises the mutation N106T in SEQ ID NO: 5 (e.g., as in SEQ ID NO: 12).

In some embodiments, the anti-CD3 heavy chain variable region (VH) comprises an N103H and/or N106T mutation in SEQ ID NO: 5 (as in SEQ ID NO: 18).

In some embodiments, the anti-CD3 heavy chain variable region (VH) comprises an N103M and/or N106T mutation in SEQ ID NO: 5 (as in SEQ ID NO: 20).

In some embodiments, the anti-CD3 heavy chain variable region (VH) comprises an N103Q and/or N106T mutation in SEQ ID NO: 5 (e.g., as in SEQ ID NO: 22).

In some embodiments, the VL2 comprises the amino acid sequence selected from the group consisting of SEQ ID NO:14, SEQ ID NO:16, SEQ ID NO:24, and SEQ ID NO:29. In some embodiments, the VH2 comprises an amino acid sequence selected from the group consisting of SEQ ID NO:12, SEQ ID NO:18, SEQ ID NO:20, and SEQ ID NO:22.

In some embodiments, the bispecific humanized anti-FLT3/anti-CD3 antibodies or fragments thereof comprise (i) a first light chain (VL1) variable region comprising the amino acid sequence of SEQ ID NO: 4 or SEQ ID NO: 6; and/or (ii) a first light chain variable region (VH1) comprising the amino acid sequence of SEQ ID NO:5 or SEQ ID NO:7; wherein the VL1 and the VH1 bind to human CD3; and further comprises a VL2 and VH2 that bind to human FLT3.

In some embodiments, the bispecific humanized anti-FLT3/anti-CD3 antibodies or fragments thereof comprise (i) a first light chain variable region (VL1) comprising the amino acid sequence of SEQ ID NO: 4; and/or (ii) a first light chain variable region (VH1) comprising the amino acid sequence of SEQ ID NO:5; wherein the VL1 and the VH1 bind to human CD3; and further comprises a VL2 and VH2 that bind to human FLT3.

In some embodiments, the bispecific humanized anti-FLT3/anti-CD3 antibodies or fragments thereof comprise (i) a first light chain variable region (VL1) comprising the amino acid sequence of SEQ ID NO: 6; and/or (ii) a first light chain variable region (VH1) comprising the amino acid sequence of SEQ ID NO:7; wherein the VL1 and the VH1 bind to human CD3; and further comprises a VL2 and VH2 that bind to human FLT3.

In some embodiments, the bispecific humanized anti-FLT3/anti-CD3 antibody or fragment comprises a variable region sequence, wherein the variable region comprises the amino acid sequences in the following order of SEQ ID NO: 9, the linker of SEQ ID NO: 60, SEQ ID NO: 10, the linker of SEQ ID NO: 26, and SEQ ID NO:5. In some embodiments, the bispecific humanized antibody comprises the amino acid sequence of SEQ ID NO 53.

In some embodiments, the bispecific humanized anti-FLT3/anti-CD3 antibody or fragment comprises a variable region sequence, wherein the variable region comprises the amino acid sequences in the following order of SEQ ID NO: 9, the linker of SEQ ID NO: 60, SEQ ID NO: 10, the linker of SEQ ID NO: 26, and SEQ ID NO:12. In some embodiments, the bispecific humanized antibody comprises the amino acid sequence of SEQ ID NO 54.

In some embodiments, the bispecific humanized anti-FLT3/anti-CD3 antibody or fragment comprises a variable region sequence, wherein the variable region comprises the amino acid sequences in the following order of SEQ ID NO: 9, the linker of SEQ ID NO: 60, SEQ ID NO: 10, the linker of SEQ ID NO: 26, and SEQ ID NO:18. In some embodiments, the bispecific humanized antibody comprises the amino acid sequence of SEQ ID NO 55.

In some embodiments, the bispecific humanized anti-FLT3/anti-CD3 antibody or fragment comprises a variable region sequence, wherein the variable region comprises the amino acid sequences in the following order of SEQ ID NO. 9, the linker of SEQ ID NO: 60, SEQ ID NO. 10, the linker of SEQ ID NO: 26, and SEQ ID NO:20. In some embodiments, the bispecific humanized antibody comprises the amino acid sequence of SEQ ID NO 56.

In some embodiments, the bispecific humanized anti-FLT3/anti-CD3 antibody or fragment comprises a variable region sequence, wherein the variable region comprises the amino acid sequences in the following order of SEQ ID NO: 9, the linker of SEQ ID NO. 60, SEQ ID NO: 10, the linker of SEQ ID NO: 26, and SEQ ID NO:22. In some embodiments, the bispecific humanized antibody comprises the amino acid sequence of SEQ ID NO 57.

### LC and HC

In some embodiments, the bispecific anti-FLT3/anti-CD3 antibodies described herein comprise a heavy chain (HC) and a light chain (LC). In some embodiments, the bispecific humanized anti-FLT3/anti-CD3 antibodies or fragments described herein comprise a heavy chain (HC). In some embodiments, the bispecific anti-FLT3/anti-CD3 antibodies or fragments described herein comprise a light chain (LC).

In some embodiments, the LC comprises a constant domain (such as any constant domain known in the art or described herein). In some embodiments, provided herein are anti-FLT3/anti-CD3 antibodies or fragments thereof comprising a light chain constant domain comprising an amino acid sequence of SEQ ID NO: 58. In some embodiments, the light chain constant domain comprises an amino acid sequence having at least 80%, at least 85%, at least 90%, at least 95%, or at least 99% sequence identity to SEQ ID NO:58 (e.g., at least 95% identity).

In some embodiments, the HC comprises a constant domain (such as any constant domain known in the art or described herein). In some embodiments, the HC comprises an Fc region (such as any Fc region known in the art or described herein). In some embodiments, the Fc region is an IgG (e.g., a human IgG). In some embodiments, the Fc region is an IgG1 (e.g., a human IgG1), an IgG2 (e.g., a human IgG2), an IgG3 (e.g., a human IgG3), or an IgG4 (e.g., a human IgG4). In some embodiments, the Fc region is an IgG1 (e.g., a human IgG1). In some embodiments, the Fc region comprises an amino acid sequence having at least 80%, at least 85%, at least 90%, at least 95%, or at least 99% sequence identity to human IgG1 (e.g., at least 95% identity).

In some embodiments, provided herein are anti-FLT3/anti-CD3 bispecific antibodies or fragments thereof comprising a heavy chain constant domain comprising an amino acid sequence of SEQ ID NO: 59. In some embodiments, the heavy chain constant domain comprises an amino acid sequence having at least 80%, at least 85%, at least 90%, at least 95%, or at least 99% sequence identity to SEQ ID NO:59 (e.g., at least 95% identity). In some embodiments, provided herein are anti-FLT3/anti-CD3 bispecific antibodies or fragments thereof comprising a heavy chain constant domain comprising one or more (e.g., 2 or 3) of the following amino acid mutations: I136A, S137A, and H318A, in SEQ ID NO: 59 (e.g., as in SEQ ID NO:27). In some embodiments, provided herein are anti-FLT3/anti-CD3 bispecific antibodies or fragments thereof comprising a heavy chain constant domain comprising an amino acid sequence of SEQ ID NO: 27.

In some embodiments, provided herein are anti-FLT3/anti-CD3 bispecific antibodies or fragments thereof comprising a light chain comprising an amino acid sequence selected from any one of SEQ ID NOs: 8, 15, 17, 25, and 30. In some embodiments, provided herein are anti-FLT3/anti-CD3 bispecific antibodies or fragments thereof comprising a light chain comprising the amino acid sequence of SEQ ID NOs: 8. In some embodiments, provided herein are anti-FLT3/anti-CD3 bispecific antibodies or fragments thereof comprising a light chain comprising the amino acid sequence of SEQ ID NO: 15. In some embodiments, provided herein are anti-FLT3/anti-CD3 bispecific antibodies or fragments thereof comprising a light chain comprising the amino acid sequence of SEQ ID NOs: 17. In some embodiments, provided herein are anti-FLT3/anti-CD3 bispecific antibodies or fragments thereof comprising a light chain comprising the amino acid sequence of SEQ ID NOs: 25. In some embodiments, provided herein are anti-FLT3/anti-CD3 bispecific antibodies or fragments thereof comprising a light chain comprising the amino acid sequence of SEQ ID NOs: 30.

In some embodiments, the light chain comprises any variable light chain (VL) described herein and the light chain constant domain comprising SEQ ID NO. 58. In some embodiments, the light chain comprises the variable light chain of SEQ ID NO: 4, and the light chain constant domain comprising SEQ ID NO: 58. In some embodiments, the light chain comprises SEQ ID NO: 8. In some embodiments, the light chain comprises the variable light chain of SEQ ID NO: 14, and the light chain constant domain comprising SEQ ID NO: 58. In some embodiments, the light chain comprises SEQ ID NO: 15. In some embodiments, the light chain comprises the variable light chain of SEQ ID NO: 16, and the light chain constant domain comprising SEQ ID NO: 58. In some embodiments, the light chain comprises SEQ ID NO: 17. In some embodiments, the light chain comprises the variable light chain of SEQ ID NO: 24, and the light chain constant domain comprising SEQ ID NO: 58. In some embodiments, the light chain comprises SEQ ID NO: 25. In some embodiments, the light chain comprises the variable light chain of SEQ ID NO: 29, and the light chain constant domain comprising SEQ ID NO: 58. In some embodiments, the light chain comprises SEQ ID NO: 30.

In some embodiments, provided herein are anti-FLT3/anti-CD3 bispecific antibodies or fragments thereof comprising a heavy chain comprising an amino acid sequence selected from any one of SEQ ID NOs: 53, 54, 55, 56, or 57. In some embodiments, provided herein are anti-FLT3/anti-CD3 bispecific antibodies or fragments thereof comprising a heavy chain comprising the amino acid sequence of SEQ ID NO: 53. In some embodiments, provided herein are anti-FLT3/anti-CD3 bispecific antibodies or fragments thereof comprising a heavy chain comprising the amino acid sequence of SEQ ID NO: 54. In some embodiments, provided herein are anti-FLT3/anti-CD3 bispecific antibodies or fragments thereof comprising a heavy chain comprising the amino acid sequence of SEQ ID NO: 55. In some embodiments, provided herein are bispecific anti-FLT3/anti-CD3 antibodies or fragments thereof comprising a heavy chain comprising the amino acid sequence of SEQ ID NO: 56. In some embodiments, provided herein are bispecific anti-FLT3/anti-CD3 antibodies or fragments thereof comprising a heavy chain comprising the amino acid sequence of SEQ ID NO: 57.

In some embodiments, the heavy chain comprises any VH described herein and the heavy chain constant domain comprising SEQ ID NO: 59. In some embodiments, the heavy chain comprises any VH described herein and the heavy chain constant domain comprising SEQ ID NO: 27.

In some embodiments, the bispecific antibody or fragment contemplated herein comprises a heavy chain comprising the following domains in the following order. FLT3 VL-linker-FLT3 VH-linker-CD3 VH-HC constant domain. In some embodiments, the heavy chain comprises the following amino acid sequences in the following order: SEQ ID NO: 9-a linker-SEQ ID NO: 10-a linker-SEQ ID NO:5-SEQ ID NO:59. In some embodiments, the heavy chain comprises the following amino acid sequences in the following order: SEQ ID NO: 9-a linker-SEQ ID NO: 10-a linker-SEQ ID NO:12-SEQ ID NO:59. In some embodiments, the heavy chain comprises the following amino acid sequences in the following order: SEQ ID NO: 9-a linker-SEQ ID NO: 10-a linker-SEQ ID NO:18-SEQ ID NO:59. In some embodiments, the heavy chain comprises the following amino acid sequences in the following order: SEQ ID NO: 9-a linker-SEQ ID NO: 10-a linker-SEQ ID NO:20-SEQ ID NO:59. In some embodiments, the heavy chain comprises the following amino acid sequences in the following order: SEQ ID NO: 9-a linker-SEQ ID NO: 10-a linker-SEQ ID NO:22-SEQ ID NO:59. In some embodiments, the heavy chain comprises the following amino acid sequences in the following order: SEQ ID NO: 9-a linker-SEQ ID NO: 10-a linker-SEQ ID NO:5-SEQ ID NO:27. The linker can be any linker known in the art or described herein. In some embodiments, the linker has SEQ ID NO:60. In some embodiments, the linker has SEQ ID NO:26. In some embodiments, the first linker (order-wise) has SEQ ID NO:60 and the second linker has SEQ ID NO:26. In some embodiments, the heavy chain comprises the following amino acid sequences in the following order: SEQ ID NO: 9-the linker of SEQ ID NO: 60-SEQ ID NO: 10-the linker of SEQ ID NO: 26-SEQ ID NO:5-SEQ ID NO:59. In some embodiments, any of the domains indicated above can be replaced by another version (e.g., a mutated version) of the same domain described herein.

In some embodiments, the bispecific antibody or fragment contemplated herein comprises a light chain comprising the following domains in the following order: CD3 VL-LC constant domain. In some embodiments, the light chain comprises the following amino acid sequences in the following order: SEQ ID NO:4-SEQ ID NO: 58. In some embodiments, the light chain comprises the following amino acid sequences in the following order: SEQ ID NO:14-SEQ ID NO: 58. In some embodiments, the light chain comprises the following amino acid sequences in the following order: SEQ ID NO:16-SEQ ID NO: 58. In some embodiments, the light chain comprises the following amino acid sequences in the following order: SEQ ID NO:24-SEQ ID NO: 58. In some embodiments, the light chain comprises the following amino acid sequences in the following order: SEQ ID NO:29-SEQ ID NO. 58. In some embodiments, any of the domains indicated above can be replaced by another version (e.g., a mutated version) of the same domain described herein.

In some embodiments, the bispecific humanized antibody comprises a heavy chain, wherein the heavy chain comprises the amino acid sequences in the following order: SEQ ID NO: 9, the linker of SEQ ID NO: 60, SEQ ID NO: 10, the linker of SEQ ID NO: 26, SEQ ID NO:5, and SEQ ID NO:59.

In some embodiments, provided herein are bispecific humanized anti-FLT3/anti-CD3 antibodies or fragments thereof comprising (i) a HC comprising the amino acid sequence of SEQ ID NO: 11, and/or (ii) a LC comprising the amino acid sequence of SEQ ID NO: 8. In some embodiments, provided herein are bispecific humanized anti-FLT3/anti-CD3 antibodies or fragments thereof comprising (i) a HC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 11, and/or (ii) a LC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 8. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a HC comprising an amino acid sequence having at least 95%, at least 96%, at least 97%, at least 98%, or at least 99% identity to the amino acid sequence of SEQ ID NO: 11, with at least 95% (or at least 96%, 97%, 98%, 99% or 100%) identity in the framework regions and at least 97% (or at least 98%, 99% or 100% identity) in the CDR regions, and/or (ii) a LC comprising an amino acid sequence having at least 95%, at least 96%, at least 97%, at least 98%, or at least 99% identity to the amino acid sequence of SEQ ID NO: 8, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof comprising both the HC and the LC comprising the sequences specified in this paragraph.

In some embodiments, the bispecific humanized antibody comprises a heavy chain, wherein the heavy chain comprises the amino acid sequences in the following order: SEQ ID NO. 9, the linker of SEQ ID NO: 60, SEQ ID NO: 10, the linker of SEQ ID NO: 26, SEQ ID NO:12, and SEQ ID NO:59.

In some embodiments, provided herein are bispecific humanized anti-FLT3/anti-CD3 antibodies or fragments thereof comprising (i) a HC comprising the amino acid sequence of SEQ ID NO: 13, and/or (ii) a LC comprising the amino acid sequence of SEQ ID NO: 8. In some embodiments, provided herein are bispecific humanized anti-FLT3/anti-CD3 antibodies or fragments thereof comprising (i) a HC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 13, and/or (ii) a LC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 8. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a HC comprising an amino acid sequence having at least 95%, at least 96%, at least 97%, at least 98%, or at least 99% identity to the amino acid sequence of SEQ ID NO: 13, with at least 95% (or at least 96%, 97%, 98%, 99% or 100%) identity in the framework regions and at least 97% (or at least 98%, 99% or 100% identity) in the CDR regions, and/or (ii) a LC comprising an amino acid sequence having at least 95%, at least 96%, at least 97%, at least 98%, or at least 99% identity to the amino acid sequence of SEQ ID NO: 8, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof comprising both the HC and the LC comprising the sequences specified in this paragraph.

In some embodiments, provided herein are bispecific humanized anti-FLT3/anti-CD3 antibodies or fragments thereof comprising (i) a HC comprising the amino acid sequence of SEQ ID NO: 13, and/or (ii) a LC comprising the amino acid sequence of SEQ ID NO: 15. In some embodiments, provided herein are bispecific humanized anti-FLT3/anti-CD3 antibodies or fragments thereof comprising (i) a HC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 13, and/or (ii) a LC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 15. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a HC comprising an amino acid sequence having at least 95%, at least 96%, at least 97%, at least 98%, or at least 99% identity to the amino acid sequence of SEQ ID NO: 13, with at least 95% (or at least 96%, 97%, 98%, 99% or 100%) identity in the framework regions and at least 97% (or at least 98%, 99% or 100% identity) in the CDR regions, and/or (ii) a LC comprising an amino acid sequence having at least 95%, at least 96%, at least 97%, at least 98%, or at least 99% identity to the amino acid sequence of SEQ ID NO: 15, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof comprising both the HC and the LC comprising the sequences specified in this paragraph.

In some embodiments, provided herein are bispecific humanized anti-FLT3/anti-CD3 antibodies or fragments thereof comprising (i) a HC comprising the amino acid sequence of SEQ ID NO: 13, and/or (ii) a LC comprising the amino acid sequence of SEQ ID NO: 17. In some embodiments, provided herein are bispecific humanized anti-FLT3/anti-CD3 antibodies or fragments thereof comprising (i) a HC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 13, and/or (ii) a LC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 17. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a HC comprising an amino acid sequence having at least 95%, at least 96%, at least 97%, at least 98%, or at least 99% identity to the amino acid sequence of SEQ ID NO: 13, with at least 95% (or at least 96%, 97%, 98%, 99% or 100%) identity in the framework regions and at least 97% (or at least 98%, 99% or 100% identity) in the CDR regions, and/or (ii) a LC comprising an amino acid sequence having at least 95%, at least 96%, at least 97%, at least 98%, or at least 99% identity to the amino acid sequence of SEQ ID NO: 17, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof comprising both the HC and the LC comprising the sequences specified in this paragraph.

In some embodiments, the bispecific humanized antibody comprises a heavy chain, wherein the heavy chain comprises the amino acid sequences in the following order of SEQ ID NO: 9, the linker of SEQ ID NO: 60, SEQ ID NO: 10, the linker of SEQ ID NO: 26, SEQ ID NO: 18, and SEQ ID NO:59.

In some embodiments, provided herein are bispecific humanized anti-FLT3/anti-CD3 antibodies or fragments thereof comprising (i) a HC comprising the amino acid sequence of SEQ ID NO: 19, and/or (ii) a LC comprising the amino acid sequence of SEQ ID NO: 17. In some embodiments, provided herein are bispecific humanized anti-FLT3/anti-CD3 antibodies or fragments thereof comprising (i) a HC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 19, and/or (ii) a LC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 17. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a HC comprising an amino acid sequence having at least 95%, at least 96%, at least 97%, at least 98%, or at least 99% identity to the amino acid sequence of SEQ ID NO: 19, with at least 95% (or at least 96%, 97%, 98%, 99% or 100%) identity in the framework regions and at least 97% (or at least 98%, 99% or 100% identity) in the CDR regions, and/or (ii) a LC comprising an amino acid sequence having at least 95%, at least 96%, at least 97%, at least 98%, or at least 99% identity to the amino acid sequence of SEQ ID NO: 17, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof comprising both the HC and the LC comprising the sequences specified in this paragraph.

In some embodiments, the bispecific humanized antibody comprises a heavy chain, wherein the heavy chain comprises the amino acid sequences in the following order of SEQ ID NO. 9, the linker of SEQ ID NO: 60, SEQ ID NO: 10, the linker of SEQ ID NO: 26, SEQ ID NO: 20, and SEQ ID NO:59.

In some embodiments, provided herein are bispecific humanized anti-FLT3/anti-CD3 antibodies or fragments thereof comprising (i) a HC comprising the amino acid sequence of SEQ ID NO: 21, and/or (ii) a LC comprising the amino acid sequence of SEQ ID NO: 17. In some embodiments, provided herein are bispecific humanized anti-FLT3/anti-CD3 antibodies or fragments thereof comprising (i) a HC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 21, and/or (ii) a LC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 17. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a HC comprising an amino acid sequence having at least 95%, at least 96%, at least 97%, at least 98%, or at least 99% identity to the amino acid sequence of SEQ ID NO: 21, with at least 95% (or at least 96%, 97%, 98%, 99% or 100%) identity in the framework regions and at least 97% (or at least 98%, 99% or 100% identity) in the CDR regions, and/or (ii) a LC comprising an amino acid sequence having at least 95%, at least 96%, at least 97%, at least 98%, or at least 99% identity to the amino acid sequence of SEQ ID NO: 17, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof comprising both the HC and the LC comprising the sequences specified in this paragraph.

In some embodiments, the bispecific humanized antibody comprises a heavy chain, wherein the heavy chain comprises the amino acid sequences in the following order of SEQ ID NO: 9, the linker of SEQ ID NO: 60, SEQ ID NO: 10, the linker of SEQ ID NO: 26, SEQ ID NO: 22, and SEQ ID NO:59.

In some embodiments, provided herein are bispecific humanized anti-FLT3/anti-CD3 antibodies or fragments thereof comprising (i) a HC comprising the amino acid sequence of SEQ ID NO: 23, and/or (ii) a LC comprising the amino acid sequence of SEQ ID NO: 17. In some embodiments, provided herein are bispecific humanized anti-FLT3/anti-CD3 antibodies or fragments thereof comprising (i) a HC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 23, and/or (ii) a LC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 17. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a HC comprising an amino acid sequence having at least 95%, at least 96%, at least 97%, at least 98%, or at least 99% identity to the amino acid sequence of SEQ ID NO: 23, with at least 95% (or at least 96%, 97%, 98%, 99% or 100%) identity in the framework regions and at least 97% (or at least 98%, 99% or 100% identity) in the CDR regions, and/or (ii) a LC comprising an amino acid sequence having at least 95%, at least 96%, at least 97%, at least 98%, or at least 99% identity to the amino acid sequence of SEQ ID NO: 17, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof comprising both the HC and the LC comprising the sequences specified in this paragraph.

In some embodiments, the bispecific humanized antibody comprises a heavy chain, wherein the heavy chain comprises the amino acid sequences in the following order of SEQ ID NO: 9, the linker of SEQ ID NO: 60, SEQ ID NO: 10, the linker of SEQ ID NO: 26, SEQ ID NO: 5, and SEQ ID NO:27.

In some embodiments, provided herein are bispecific humanized anti-FLT3/anti-CD3 antibodies or fragments thereof comprising (i) a HC comprising the amino acid sequence of SEQ ID NO: 28, and/or (ii) a LC comprising the amino acid sequence of SEQ ID NO: 25. In some embodiments, provided herein are bispecific humanized anti-FLT3/anti-CD3 antibodies or fragments thereof comprising (i) a HC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 28, and/or (ii) a LC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 25. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a HC comprising an amino acid sequence having at least 95%, at least 96%, at least 97%, at least 98%, or at least 99% identity to the amino acid sequence of SEQ ID NO: 28, with at least 95% (or at least 96%, 97%, 98%, 99% or 100%) identity in the framework regions and at least 97% (or at least 98%, 99% or 100% identity) in the CDR regions, and/or (ii) a LC comprising an amino acid sequence having at least 95%, at least 96%, at least 97%, at least 98%, or at least 99% identity to the amino acid sequence of SEQ ID NO: 25, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof comprising both the HC and the LC comprising the sequences specified in this paragraph.

In some embodiments, provided herein are bispecific humanized anti-FLT3/anti-CD3 antibodies or fragments thereof comprising (i) a HC comprising the amino acid sequence of SEQ ID NO: 28, and/or (ii) a LC comprising the amino acid sequence of SEQ ID NO: 30. In some embodiments, provided herein are bispecific humanized anti-FLT3/anti-CD3 antibodies or fragments thereof comprising (i) a HC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 28, and/or (ii) a LC comprising an amino acid sequence having at least 85%, at least 90%, at least 91%, at least 92%, at least 93%, at least 94%, at least 95%, at least 96%, at least 97%, at least 98%, or at least 99% identity to the amino acid sequence of SEQ ID NO: 30. In certain embodiments, substitutions, insertions or deletions in these sequences occur in regions outside the CDRs (i.e., in the framework regions). In certain embodiments, provided herein are bispecific humanized anti-FLT3/anti-CD3 antibodies or fragments thereof comprising (i) a HC comprising an amino acid sequence having at least 95%, at least 96%, at least 97%, at least 98%, or at least 99% identity to the amino acid sequence of SEQ ID NO: 28, with at least 95% (or at least 96%, 97%, 98%, 99% or 100%) identity in the framework regions and at least 97% (or at least 98%, 99% or 100% identity) in the CDR regions, and/or (ii) a LC comprising an amino acid sequence having at least 95%, at least 96%, at least 97%, at least 98%, or at least 99% identity to the amino acid sequence of SEQ ID NO: 30, with at least 95% (or at least 96%, 97%, 98%, 99% or 100%) identity in the framework regions and at least 97% (or at least 98%, 99% or 100% identity) in the CDR regions. In some embodiments, provided herein are bispecific humanized anti-FLT3/anti-CD3 antibodies or fragments thereof comprising both the HC and the LC comprising the sequences specified in this paragraph

### Antigen Binding Fragments of Antibodies

In some embodiments, provided herein are fragments of the humanized anti-FLT3 and anti-CD3 antibodies described herein. In certain embodiments, provided herein are scFv fragments comprising any VH and/or VL described herein, including any VH and VL pairs described herein (including those mediating binding to FLT3 and those mediating binding to CD3). Methods of making single chain variable fragment antibodies are known in the art. For example, an scFv of antibody can be made by fusing a heavy chain variable region (VH) with a light chain variable region via a short peptide linker. Suitable short peptide linkers are known in the art, and exemplary linkers are described herein.

In some embodiments, provided herein are fragments of the antibodies described herein comprising any of the VH and/or VL mediating binding to FLT3 (including any VH and VL pairs described herein) and a VH mediating binding to CD3. In some embodiments, provided herein are fragments of the antibodies described herein comprising in the following order: a VH and a VL mediating binding to FLT3 (including any VH and VL pairs described herein, bound by any linker described herein or known in the art), a linker (such as any linker described herein or known in the art) and a VH mediating binding to CD3 (such as any anti-CD3 VH described herein).

In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity, to an amino acid sequence selected from any one of SEQ ID NOs: 3 and 52. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence selected from any one of SEQ ID NOs: 3 and 52, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising the amino acid sequence of SEQ ID NO: 3. In certain embodiments, provided herein is an anti-FLT3 scFv fragment comprising the amino acid sequence of SEQ ID NO: 52.

In some embodiments, provided herein are anti-FLT3/CD3 fragments comprising an amino acid sequence having at least 85%, at least 90%, at least 91%, at least 92%, at least 93%, at least 94%, at least 95%, at least 96%, at least 97%, at least 98%, or at least 99% identity, to an amino acid sequence selected from any one of SEQ ID NOs: 53, 54, 55, 56, and 57. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence selected from any one of SEQ ID NOs: 53, 54, 55, 56, and 57, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3/CD3 fragment comprising the amino acid sequence of SEQ ID NO: 53. In certain embodiments, provided herein is an anti-FLT3/CD3 fragment comprising the amino acid sequence of SEQ ID NO: 54. In certain embodiments, provided herein is an anti-FLT3/CD3 fragment comprising the amino acid sequence of SEQ ID NO. 55. In certain embodiments, provided herein is an anti-FLT3/CD3 fragment comprising the amino acid sequence of SEQ ID NO: 56. In certain embodiments, provided herein is an anti-FLT3/CD3 fragment comprising the amino acid sequence of SEQ ID NO: 57.

Linkers that can be Used to Join Antibody Fragments, e.g., in scFvs

In some embodiments, the disclosure provides anti-FLT3 and/or anti-CD3 single-chain variable fragments (e.g. scFv) comprising one or more linkers linking a VH and a VL. A “linker” is a functional group which covalently attaches two or more polypeptides or nucleic acids so that they are connected to one another. The linker can be any linker known in the art. In some embodiments, the linker comprises hydrophilic amino acids. In some embodiments, the linker comprises glycine and serine.

In some embodiments, the linker has the formula (Gly₃₋₄-Ser)₁₋₄. In some embodiments, the linker is a Gly₄Ser linker, repeated from 1 to 4 times. In some embodiments, the linker is a Gly₃Ser linker, repeated from 1 to 4 times. In some embodiments, the linker comprises Gly₄Ser and Gly₃Ser, each repeated from 1 to 4 times.

In certain embodiments, the linker is 4 to 25 amino acids in length. In certain embodiments, the linker is 4 to 21 amino acids in length. In some embodiments, the linker is 4, 5, 6, 7, 8, 9, 10, 11, 12, 13, 14, 15, 16, 17, 18, 19, 20, or 21 amino acids in length. In some embodiments, the linker is 5 amino acids in length. In some embodiments, the linker is 10 amino acids in length. In some embodiments, the linker is 15 amino acids in length. In some embodiments, the linker is 19 amino acids in length. In some embodiments, the linker is 20 amino acids in length.

In some embodiments, the linker comprises the amino acid sequence of SEQ ID NO:60. In some embodiments, the linker comprises the amino acid sequence of SEQ ID 61. In some embodiments, the linker comprises the amino acid sequence of SEQ ID NO:26.

In some embodiments, a bispecific anti-FLT3/CD3 antibody or antigen binding fragment thereof that binds to human FLT3 and human CD3 comprises:
(i) a first light chain variable region (VL1); and
(ii) a first heavy chain variable region (VH1), wherein the VL1 and the VH1 bind to human FLT3; and further comprising a second VL (VL2) and a second VH (VH2) that bind to human CD3. In some embodiments, the VL1 is joined to the VH1 by a first linker, and wherein the VH1 is joined to the VH2 by a second linker. In some embodiments, the C-terminus of the VL1 is joined to the N-terminus of the VH1 by a first linker. In some embodiments, the C-terminus of the VH1 is joined to the N-terminus of the VH2 by a second linker. In some embodiments, the first linker has the formula (Gly₄-Ser)₄. In some embodiments, the second linker has the formula (Gly₄-Ser)₃. In some embodiments, the first linker has a SEQ ID NO:60 or 61, and/or the second linker has a SEQ ID NO:26.

In some embodiments, a bispecific humanized anti-FLT3/CD3 antibody or antigen binding fragment thereof that binds to human FLT3 and human CD3 comprises:
(i) a first light chain variable region (VL1); and
(ii) a first heavy chain variable region (VH1), wherein the VL1 and the VH1 bind to human FLT3 and are joined by a first linker comprising the formula (Gly₄-Ser)₄; and further comprising a second VL (VL2) and a second VH (VH2) that bind to human CD3 and are joined by a second linker comprising the formula (Gly₄-Ser)₃.

### Additional Anti-FLT3 Anti-CD3 Bispecific Antibodies, Fragments and Characteristics

In some embodiments, the bispecific anti-FLT3/anti-CD3 antibody or fragment described herein is a purified antibody. In some of these embodiments, the antibody or fragment is humanized.

In some embodiments, described herein are anti-FLT3/anti-CD3 bispecific antibodies, wherein the antibody is an immunoglobulin comprising any VH and VL regions described herein. In some embodiments, the bispecific antibodies described herein comprise a heavy chain (HC). In some embodiments, the heavy chain (HC) of an antibody described herein comprises an Fc region. The immunoglobulin molecules that can be used are of any type (e.g., IgG, IgE, IgM, IgD, IgY, IgA). The immunoglobulin molecules that can be used are of any class (e.g., IgG1, IgG2, IgG3, IgG4, IgA1, IgA2). The immunoglobulin molecules that can be used are of any subclass. In some embodiments, the immunoglobulin (i.e. the Fc region) of the HC is IgG. In some embodiments, the IgG of the HC is human IgG1. In some embodiments, the IgG of the HC is human IgG2. In some embodiments, the IgG of the HC is human IgG3. In some embodiments, the IgG of the HC is human IgG4.

In some embodiments, described herein are single domain anti-FLT3/anti-CD3 bispecific antibodies or fragments, having only the heavy chain or only the light chain binding to FLT3 (comprising any VH or VL described herein). Is some embodiments, described herein are single domain anti-FLT3/anti-CD3 bispecific antibodies or fragments having only the heavy chain binding FLT3 (comprising any VH described herein).

In some embodiments, described herein are single domain anti-FLT3/anti-CD3 bispecific antibodies or fragments, having only the heavy chain or only the light chain binding CD3 (comprising any VH or VL described herein). Is some embodiments, described herein are single domain anti-FLT3/anti-CD3 bispecific antibodies or fragments having only the heavy chain binding CD3 (comprising any VH described herein).

In some embodiments, described herein are antigen-binding fragments of anti-FLT3/anti-CD3 bispecific antibodies, which include, without limitation, an Fv fragment, a Fab fragment, a F(ab′) fragment, a F(ab′)₂ fragment or a disulfide-linked Fv (sdFv). In some embodiments, the antigen-binding fragment of an anti-FLT3/anti-CD3 bispecific antibody is an Fv fragment. In some embodiments, the antigen-binding fragment of an anti-FLT3/anti-CD3 bispecific antibody is a Fab fragment. In some embodiments, the antigen-binding fragment of an anti-FLT3/anti-CD3 bispecific antibody is a F(ab′) fragment. In some embodiments, the antigen-binding fragment of an anti-FLT3/anti-CD3 bispecific antibody is a F(ab′)₂ fragment. In some embodiments, the antigen-binding fragment of an anti-FLT3/anti-CD3 bispecific antibody is a disulfide-linked Fv (sdFv).

In some embodiments, described herein are chimeric anti-FLT3/anti-CD3 bispecific antibodies or antigen-binding fragments thereof, where the chimeric antibody has any of the CDRs described herein, murine variable region and a constant region of another species (e.g., human).

In some embodiments, described herein are anti-FLT3/anti-CD3 bispecific antibodies and fragments thereof which have a binding affinity for a FLT3 protein with an EC50 from about 0.1 nM to 100 nM, 0.5 nM to 50 nM, or 1 nM to 10 nM. In some embodiments, described herein are anti-FLT3/anti-CD3 bispecific antibodies and fragments thereof which have a binding affinity for a FLT3 protein with an EC50 that is less than about 100 nM, less than about 75 nM, less than about 50 nM, less than about 25 nM, less than about 10 nM, less than about 5 nM, less than about 3 nM, less than about 2 nM, or less than about 1 nM. In some embodiments, described herein are anti-FLT3/anti-CD3 bispecific antibodies and fragments thereof which have a binding affinity for a FLT3 protein with an EC50 that is less than 15 nM, less than 10 nM, less than 5 nM or less than 2.5 nM.

In some embodiments, described herein are anti-FLT3/anti-CD3 bispecific antibodies and fragments thereof which have a binding affinity for a CD3 protein with an EC50 from about 0.1 nM to 100 nM, 0.5 nM to 50 nM, or 1 nM to 10 nM. In some embodiments, described herein are anti-FLT3/anti-CD3 bispecific antibodies and fragments thereof which have a binding affinity for a CD3 protein with an EC50 that is less than about 100 nM, less than about 75 nM, less than about 50 nM, less than about 25 nM, less than about 10 nM, less than about 5 nM, less than about 3 nM, less than about 2 nM, or less than about 1 nM. In some embodiments, described herein are anti-FLT3/anti-CD3 bispecific antibodies and fragments thereof which have a binding affinity for a CD3 protein with an EC50 that is less than 15 nM, less than 10 nM, less than 5 nM or less than 2.5 nM.

In some embodiments, the anti-FLT3/anti-CD3 bispecific antibodies and fragments thereof described herein do not compete with FLT3 ligand for binding to FLT3.

In some embodiments, the anti-FLT3/anti-CD3 bispecific antibodies and fragments thereof described herein are bivalent on both FLT3 and CD3. In some embodiments, the anti-FLT3/anti-CD3 bispecific antibodies and fragments thereof described herein bind bivalently to FLT3 and/or CD3. In some embodiments, the anti-FLT3/anti-CD3 bispecific antibodies and fragments thereof described herein bind with 6 CDRs (3 VL and 3 VH) to FLT3 and/or 6 CDRs (3 VL and 3 VH) to CD3, where the CDRs can be any CDRs described herein.

In some embodiments, the anti-FLT3/anti-CD3 bispecific antibodies and fragments thereof described herein have a half-life of about 1 to about 14 days in a human. In some embodiments, the anti-FLT3/anti-CD3 bispecific antibodies and fragments thereof described herein have a half-life of about 2 days to about 12 days in a human. In some embodiments, the anti-FLT3/anti-CD3 bispecific antibodies and fragments thereof described herein have a half-life of about 3 days to about 10 days in a human. In some embodiments, the anti-FLT3/anti-CD3 bispecific antibodies and fragments thereof described herein have a half-life of about 4 days to about 8 days in a human. In some embodiments, the anti-FLT3/anti-CD3 bispecific antibodies and fragments thereof described herein have a half-life of about 4 days to about 7 days in a human. In some embodiments, the anti-FLT3/anti-CD3 bispecific antibodies and fragments thereof described herein have a half-life of about 5 days to about 7 days in a human. In some embodiments, the anti-FLT3/anti-CD3 bispecific antibodies and fragments thereof described herein have a half-life of about 5 days to about 6 days in a human. In some embodiments, the anti-FLT3/anti-CD3 bispecific antibodies and fragments thereof described herein have a half-life of more than 1 day in a human. In some embodiments, the anti-FLT3/anti-CD3 bispecific antibodies and fragments thereof described herein have a half-life of more than 2 days in a human. In some embodiments, the anti-FLT3/anti-CD3 bispecific antibodies and fragments thereof described herein have a half-life of more than 3 days in a human. In some embodiments, the anti-FLT3/anti-CD3 bispecific antibodies and fragments thereof described herein have a half-life of more than 4 days in a human. In some embodiments, the anti-FLT3/anti-CD3 bispecific antibodies and fragments thereof described herein have a half-life of more than 5 days in a human. In some embodiments, the anti-FLT3/anti-CD3 bispecific antibodies and fragments thereof described herein have a half-life of more than 6 days in a human. In some embodiments, the anti-FLT3/anti-CD3 bispecific antibodies and fragments thereof described herein have a half-life of less than 14 days in a human. In some embodiments, the anti-FLT3/anti-CD3 bispecific antibodies and fragments thereof described herein have a half-life of less than 12 days in a human. In some embodiments, the anti-FLT3/anti-CD3 bispecific antibodies and fragments thereof described herein have a half-life of less than 10 days in a human. In some embodiments, the anti-FLT3/anti-CD3 bispecific antibodies and fragments thereof described herein have a half-life of more than 8 days in a human. In some embodiments, the anti-FLT3/anti-CD3 bispecific antibodies and fragments thereof described herein have a half-life of more than 7 days in a human. In some embodiments, the anti-FLT3/anti-CD3 bispecific antibodies and fragments thereof described herein have a half-life of about 1 week in a human. In some embodiments, the anti-FLT3/anti-CD3 bispecific antibodies and fragments thereof described herein have a half-life of up to 2 weeks in a human.

### Nucleic Acids, Vectors and Cells

In some aspects, the disclosure provides nucleic acids encoding any of the humanized bispecific antibodies and antigen-binding fragments thereof described herein. In some aspects, the disclosure provides a vector comprising a nucleic acid encoding any of the humanized bispecific antibodies and antigen-binding fragments thereof described herein. Also provided are cells expressing such nucleic acids for producing any of the humanized bispecific antibodies and antigen-binding fragments thereof described herein, and methods of making such antibodies and fragments.

### Making of Antibodies

The humanized bispecific antibodies and antigen-binding fragments thereof described herein can be made by any method known in the art and/or described herein.

Methods of making monoclonal antibodies are known in the art, e.g., using hybridoma technology. See e.g., Harlow E and Lane D, Antibodies: A Laboratory Manual (Cold Spring Harbor Press, 2^{nd} ed. 1988); Hammerling G J et al., in: Monoclonal Antibodies and T-Cell Hybridomas 563 (Elsevier, NY, 1981), or in Kohler G and Milstein C, 1975, Nature 256:495; Goding J W (Ed), Monoclonal Antibodies: Principles and Practice, pp. 59-103 (Academic Press, 1986) In using hybridoma technology, a mouse or another appropriate host animal can be immunized with the target protein (e.g., FLT3) to elicit lymphocytes to produce antibodies that will specifically bind to the target protein, and then the lymphocytes are fused with myeloma cells to form a hybridoma. The hybridoma cells are then grown in a culture medium and assayed for production of antibodies. The binding specificity of antibodies produced by this method can be determined by methods known in the art, e.g., enzyme-linked immunoabsorbent assay (ELISA), immunoprecipitation or radioimmunoassay (RIA). The monoclonal antibodies can be further purified.

Monoclonal antibodies can also be made using recombinant and phage display technologies and using humanized mice. See, e.g., Brinkman U et al., 1995, J. Immunol. Methods 182:41-50; Ames R S et al., 1995, J Immunol. Methods 184.177-186; Laffleur et al., 2012, Methods Mol. Biol. 901:149-59; Persic L. et al., 1997, Gene 187:9-18.

Methods of making chimeric antibodies are known in the art. See, e.g., Morrison S L, 1985, Science 229:1202-7; Gillies S D et al., 1989, J. Immunol. Methods 125:191-202; Oi V T & Morrison S L, 1986, BioTechniques 4:214-221. When making a chimeric antibody, a variable region of one species (e.g., murine) is joined with a constant region of another species (e.g., human).

Methods of making humanized antibodies are known in the art, including without limitation by CDR grafting. See, e.g., Padlan E A (1991) Mol Immunol 28 (4/5): 489-498; Studnicka G M et al, (1994) Prot Engineering 7 (6): 805-814; and Roguska M A et al. (1994) PNAS 91:969-973; Tan P et al, (2002) J Immunol 169:1119-25; Caldas C et al, (2000) Protein Eng. 13 (5): 353-60; Morea V et al. (2000), Methods 20 (3): 267-79; Baca M et al. (1997) J Biol Chem 272 (16): 10678-84; Roguska M A et al. (1996) Protein Eng 9 (10): 895 904; Couto J R et al, (1995) Cancer Res. 55 (23 Supp): 5973s-5977s: Couto J R et al, (1995) Cancer Res 55 (8): 1717-22; Sandhu J S (1994) Gene 150 (2): 409-10, Pedersen J T et al. (1994) J Mol Biol 235 (3): 959-73).

Methods of making human antibodies are known in the art and include phage display methods using antibody libraries derived from human immunoglobulin sequences. See, e.g., International Publication Nos. WO 98/46645, WO 98/50433, WO 98/24893, WO 98/16654, WO 96/34096, WO 96/33735, and WO 91/10741.

Methods of making antibody fragments, including single chain Fv (scFv), are also known in the art. See, e.g., Abmad et al., 2012, Clinical and Developmental Immunology, doi: 10.1155/2012/980250; Wang et al., 2006, Anal. Chem. 78, 997-1004; Pansri et al., 2009, BMC Biotechnology 9:6. For example, scFvs can be constructed by fusing heavy and light chain variable regions via short polypeptide linkers (using recombinant expression techniques), and scFv antibodies having desired antigen-binding properties can be selected by methods known in the art. Further, Fab and F(ab′)₂ fragments can be produced by proteolytic cleavage of immunoglobulin molecules using papain and pepsin, respectively.

Methods of making single domain antibodies (e.g., without light chains) are also known in the art. See, e.g. Riechmann L & Muyldermans S, 1999, J Immunol. 231: 25-38; Nuttall S D et al., 2000, Curr Pharm Biotechnol 1 (3): 253-263; Muyldermans S, 2001, J Biotecnol 74 (4).277-302.

Methods of making bispecific antibodies are well-known in the art. See, e.g, Konterman, 2012, MAbs 4:182-197; Gramer et al., 2013, MAbs 5.962-973.

Methods of making mouse anti-FLT3 antibodies are described in US Patent Pub. No. 20190137464 and US Patent Pub. No. 20190389955, each of which is incorporated herein in its entirety and specifically as describing the making of mouse anti-FLT3 antibodies.

Methods of affinity maturation, optimization and mutagenesis of antibodies are well-known in the art.

Methods of recombinant production of antibodies are also known in the art. In some embodiments, for recombinant production of an anti-FLT3/anti-CD3 antibody (or an antigen-binding fragment thereof), a nucleic acid encoding the antibody (or an antigen-binding fragment thereof) is isolated and inserted into one or more vectors for expression in a host cell. In some embodiments, a method of making the anti-FLT3/anti-CD3 antibody is provided, wherein the method comprises culturing a host cell comprising a nucleic acid encoding the antibody under conditions suitable for expression of the antibody, and recovering the antibody from the host cell (or host cell culture medium) and, optionally further purifying the antibody. In some embodiments, a method of making an antigen binding fragment of the anti-FLT3/anti-CD3 antibody is provided, wherein the method comprises culturing a host cell comprising a nucleic acid encoding said fragment under conditions suitable for expression of the fragment, and recovering the fragment from the host cell (or host cell culture medium) and, optionally further purifying the fragment.

### Pharmaceutical Compositions

Provided herein are pharmaceutical compositions comprising any bispecific humanized anti-FLT3/CD3 antibody or fragment thereof described herein and a pharmaceutically acceptable carrier. Appropriate pharmaceutically acceptable carriers including, but not limited to, excipients and stabilizers are known in the art (see, e.g. Remington's Pharmaceutical Sciences (1990) Mack Publishing Co., Easton, PA).

In some embodiments, pharmaceutically acceptable carriers include but are not limited to an isotonic agent, a buffer, a suspending agent, a dispersing agent, an emulsifying agent, a wetting agent, a sequestering agent, a chelating agent, a pH buffering agent, a solubility enhancer, an antioxidant, an anesthetic, and/or an antimicrobial agent. In some embodiments, the carriers are selected from, but not limited to, one or more of water, saline, phosphate buffered saline, dextrose, glycerol, ethanol and the like, starch, lactose, sucrose, gelatin, malt, propylene, silica gel, sodium stearate, and dextrose as well as combinations thereof. In some embodiments, the pharmaceutically acceptable carriers further comprise auxiliary substances such as wetting or emulsifying agents, preservatives or buffers, which enhance the shelf life or effectiveness of the binding proteins.

In some embodiments, when administered parenterally, the pharmaceutical acceptable carriers include, but are not limited to, physiological saline or phosphate buffered saline (PBS), solutions containing agents such as glucose, polyethylene glycol, polypropylene glycol, or other agents.

In some embodiments, the pharmaceutical composition is formulated to provide rapid, sustained, or delayed release of the active ingredient after administration. Formulations for providing rapid, sustained, or delayed release of the active ingredient after administration are known in the art (Mishra, M. K. (2016). Handbook of encapsulation and controlled release. Boca Raton, CRC Press, Taylor & Francis Group, CRC Press is an imprint of the Taylor & Francis Group, an Informa business, incorporated herein by reference in its entirety).

In some embodiments, a pharmaceutical composition provided herein comprises any bispecific humanized anti-FLT3/CD3 antibody or fragment thereof described herein and one or more other therapeutic agents (e.g., an anti-cancer agent) in a pharmaceutically acceptable carrier.

In some embodiments, a pharmaceutical composition is formulated for any route of administration to a subject. In some embodiments, the pharmaceutical composition is formulated for injection and prepared as a liquid solution, suspension, emulsion, or solid form suitable for making into a solution or suspension prior to injection.

In some embodiments, the bispecific humanized anti-FLT3/CD3 antibody or fragment thereof described herein is present in the pharmaceutical composition in a therapeutically effective amount. Therapeutically effective amounts are determined by clinical techniques known in the art.

### Therapeutic Methods

In some embodiments, the methods described herein comprise administering to a subject any bispecific humanized anti-FLT3/CD3 antibody or fragment thereof described herein that binds to an FLT3 epitope of a cell (e.g., of a target cell such as HSC, HPC, dendritic cell or cancer cell). In some embodiments, the methods described herein comprise administering to a subject any bispecific humanized anti-FLT3/CD3 antibody or fragment thereof described herein that binds to a FLT3 epitope of a cancer cell (e.g., AML cell). In some embodiments, the methods described herein comprise administering to a subject any bispecific humanized anti-FLT3/CD3 antibody or fragment thereof described herein that binds to a CD3 epitope of a T-cell. In some embodiments, the methods described herein comprise administering to a subject a bispecific humanized antibody or fragment thereof described herein that binds to a CD3 epitope (e.g., on an immune cell such as a T cell) and an FLT3 epitope of a target cell (such as an target cell described herein).

### Cancer Treatment

In some embodiments, the disclosure provides methods for treating cancer comprising administering any bispecific humanized anti-FLT3/CD3 antibody or fragment thereof described herein or a pharmaceutical composition comprising such an antibody/fragment.

In some embodiments, the disclosure provides a method of treating cancer that is resistant to other cancer therapy or therapies (e.g., vaccine, chemotherapy, radiotherapy, small molecule therapy, or immunotherapy (such as treatment with another antibody). In some embodiments, the cancer is resistant to vaccine therapy. In some embodiments, the cancer is resistant to chemotherapy. In some embodiments, the cancer is resistant to radiotherapy. In some embodiments, the cancer is resistant to small molecule therapy. In some embodiments, the cancer is resistant to immunotherapy.

The methods described herein are suitable for treating cancers that are expected, known, or determined to express FLT3 on the surface of their cells.

In some embodiments, the administration of any bispecific humanized anti-FLT3/CD3 antibody or fragment thereof described herein or pharmaceutical composition thereof in accordance with the methods described herein is carried out to achieve or result in one or more of the following when administered in combination with one or more of the additional therapies described herein: (i) a decrease in cancer cell frequency or number, (ii) a reduction in the growth of the cancer or increase in the number of cancer cells, (iii) inhibition of the progression of cancer cell growth, (iv) the regression of cancer, (v) inhibition of a recurrence of the cancer, (vi) eradication of the cancer, (vii) reduction or amelioration of the severity or duration of one or more symptoms of the cancer, (viii) the inhibition of the development or onset of one or more symptoms associated with cancer, (ix) the enhancement or improvement of the therapeutic effect of another anti-cancer therapy, (x) increase in life expectancy or survival of a subject, (xi) reduction in hospitalization (e.g. length of hospitalization) in a subject, (xii) improvement in a subject's quality of life, (xiii) a reduction in mortality, (xiv) an increase in a relapse free survival or length of remission in a subject. In some embodiments, the administration is carried out to achieve or result in decrease in tumor burden.

In some embodiments, of any bispecific humanized anti-FLT3/CD3 antibody or fragment thereof described herein is effective to treat cancer in a subject (e.g., decreases tumor burden, cancer cell frequency or number, reduces cancer cell growth or proliferation, increases life expectancy or survival, eradicates cancer, or improves one or more symptoms of cancer), when used alone or in combination with another therapy.

In some embodiments, administration to a subject of a bispecific humanized antibody or fragment thereof described herein or a pharmaceutical composition described herein is effective to reduce cell frequency or number of cancer cells, or eliminate cancer cells. In some embodiments, administration to a subject of a bispecific humanized antibody or fragment thereof described herein or a pharmaceutical composition described herein, is effective to reduce the number or frequency of cancer cells by at least 30%, at least 40%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of cancer cells in the subject before administration of this therapy). In some embodiments, administration to a subject of a bispecific humanized antibody or fragment thereof described herein or a pharmaceutical composition described herein, is effective to reduce the number or frequency of cancer cells by at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of cancer cells in the subject before administration of this therapy) In some embodiments, administration to a subject of a bispecific humanized antibody or fragment thereof described herein or a pharmaceutical composition described herein, is effective to reduce the number or frequency of cancer cells by at least 75%, at least 80%, at least 85%, at least 90%, at least 95%, at least 97%, at least 98%, at least 99% or about 100%, relative to control or baseline (e.g., relative to the level of cancer cells in the subject before administration of this therapy). In some embodiments, administration to a subject of a bispecific humanized antibody or fragment thereof described herein or a pharmaceutical composition described herein, is effective to reduce the number or frequency of cancer cells by at least 90%, at least 95%, at least 97%, at least 98%, at least 99% or about 100%, relative to control or baseline (e.g., relative to the level of cancer cells in the subject before administration of this therapy).

In some embodiments, administration to a subject of a bispecific humanized antibody or fragment described herein or a pharmaceutical composition described herein is effective to increase survival of the subject. In some embodiments, administration to a subject of a bispecific humanized antibody or fragment described herein or a pharmaceutical composition described herein is effective to increase median survival of subjects relative to subjects not treated or treated with a placebo. In some embodiments, administration to a subject of a bispecific humanized antibody or fragment described herein or a pharmaceutical composition described herein is effective to increase median survival of subjects relative to subjects treated with a standard of care therapy.

Examples of the cancer cells that can be reduced in number or eliminated using the methods described herein include, without limitation, blast cells of acute myeloid leukemia (AML), lymphoblasts or leukemic blasts of acute lymphocytic leukemia (ALL), myeloblasts of chronic myeloid leukemia (CML), Blastic plasmacytoid dendritic cell neoplasm (BPDCN), and blasts of chronic lymphocytic leukemia (CLL).

According to some embodiments, the bispecific humanized antibody is effective to eliminate one or more of hematopoietic stem cells (HSC), early hematopoietic progenitors (HP), and cancer cells. In some embodiments, one or more of the HPC, HP, and cancer cells express FLT3. In some embodiments, a subject in need thereof is a patient that qualifies for, will be receiving or is receiving Bone marrow (BM)/HSC/PC transplantation. Examples of the cancer cells include, without limitation, blast cells of acute myeloid leukemia (AML), acute lymphocytic leukemia (ALL), blast-crisis phase of chronic myeloid leukemia (BC-CML) and chronic lymphocytic leukemia (CLL). According to some embodiments, the bispecific antibody is effective to condition patients undergoing bone marrow (BM)/hematopoietic stem cell (HSC) transplantation. According to some embodiments, the HSC/HP transplantation is for treating a hematological malignancy or hyperproliferative disorder, e.g., Acute Myeloid Leukemia (AML), Acute Lymphoblastic Leukemia (ALL), Chronic Lymphocytic Leukemia (CLL), Chronic Myeloid Leukemia (CML), peripheral T cell lymphoma, follicular lymphoma, diffuse large B cell lymphoma, Hodgkin lymphoma, non-Hodgkin lymphoma, neuroblastoma, non-malignant inherited and acquired marrow disorders (e.g. sickle cell anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, or congenital thrombocytopenia), multiple myeloma, or Severe Combined Immunodeficiency (SCID).

### Hematopoietic Cell Transplantation

In some embodiments, the disclosure provides methods for preparing or conditioning a subject in need thereof for hematopoietic cell transplantation. In some embodiments, a subject in need thereof is a patient that qualifies for, will be receiving or is receiving bone marrow (BM) hematopoietic stem cell and/or hematopoietic progenitor cell transplantation. In some embodiments, the subject in need of a hematopoietic cell transplantation has cancer (such as any cancer described herein).

In some embodiments, the disclosure provides methods for preparing or condition a subject in need thereof for hematopoietic cell transplantation wherein the subject is administered any bispecific humanized anti-FLT3/CD3 antibody or fragment thereof described herein or a pharmaceutical composition comprising such an antibody/fragment. In some embodiments, the method of preparing or conditioning a subject comprises administering to a subject any bispecific humanized antibody or fragment described herein that binds to a FLT3 epitope on a hematopoietic stem cell. In some embodiments, the method of preparing or conditioning a subject comprises administering to a subject any bispecific humanized antibody or fragment described herein that binds to a FLT3 epitope on a hematopoietic progenitor cell. In some embodiments, the method of preparing or conditioning a subject comprises administering to a subject any bispecific humanized antibody or fragment described herein that binds to a FLT3 epitope on a dendritic cell. In some embodiments, the method of preparing or conditioning a subject comprises administering to a subject any bispecific humanized antibody or fragment described herein that binds to a FLT3 epitope on a myeloid cell. In some embodiments, the method of preparing or conditioning a subject comprises administering to a subject any bispecific humanized antibody or fragment described herein that binds to a FLT3 epitope on a lymphoid cell.

In some embodiments, a bispecific humanized antibody or fragment described herein or a pharmaceutical composition described herein is effective to significantly reduce cell frequency or number, or eliminate, hematopoietic stem cells (HSC) and/or hematopoietic progenitor cells (HPCs) (e.g., early hematopoietic progenitors). In some embodiments, administration to a subject of a bispecific humanized antibody or fragment described herein or a pharmaceutical composition described herein is effective to reduce the number or frequency of HSCs and/or HPCs (e.g., early HPCs) by at least 30%, at least 40%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of a bispecific humanized antibody or fragment described herein or a pharmaceutical composition described herein is effective to reduce the number or frequency of HSC's and/or HPCs (e.g., early HPCs) by at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of a bispecific humanized antibody or fragment described herein or a pharmaceutical composition described herein is effective to reduce the number or frequency of HSCs and/or HPCs (e.g., early HPCs) by at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of a bispecific humanized antibody or fragment described herein or a pharmaceutical composition described herein is effective to reduce the number or frequency of HSCs and/or HPCs (e.g., early HPCs) by at least 90%, at least 95%, at least 97%, at least 98%, at least 99% or about 100%, relative to control or baseline (e.g., relative to the level of the cells in the subject before administration of this therapy). In some of these embodiments, the reduction of HSCs and/or HPCs (e.g., early HPCs) is in bone marrow of the subject being treated (e.g., in bone marrow mononuclear cells).

In some embodiments, a bispecific humanized antibody or fragment described herein or a pharmaceutical composition described herein is effective to significantly reduce cell frequency or number, or eliminate, multi-potent progenitor cells (MPPs) and/or common progenitor cells (CPs). In some embodiments, administration to a subject of a bispecific humanized antibody or fragment described herein or a pharmaceutical composition described herein is effective to reduce the number or frequency of MPPs and/or CPs by at least 30%, at least 40%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of a bispecific humanized antibody or fragment described herein or a pharmaceutical composition described herein is effective to reduce the number or frequency of MPPs and/or CPs by at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of a bispecific humanized antibody or fragment described herein or a pharmaceutical composition described herein is effective to reduce the number or frequency of MPPs and/or CPs by at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of a bispecific humanized antibody or fragment described herein or a pharmaceutical composition described herein is effective to reduce the number or frequency of MPPs and/or CPs by at least 90%, at least 95%, at least 97%, at least 98%, at least 99% or about 100%, relative to control or baseline (e.g., relative to the level of the cells in the subject before administration of this therapy). In some of these embodiments, the reduction of MPPs or CPs is in bone marrow of the subject being treated (e.g., in bone marrow mononuclear cells).

In some embodiments, the therapeutically effective amount reduces a cell population expressing one or more of (e.g., 2, 3, 4, 5, 6 or 7 of) CD34, FLT3, CD33, CD11b, CD16, CD15, and CD66b. In some embodiments, the therapeutically effective amount reduces a cell population expressing one or more of (e.g., 2, 3, 4, 5, 6 or 7 of) CD34, FLT3, CD33, CD11b, CD16, CD15, and CD66b by at least 25%, at least 30%, at least 40%, at least 50%, at least 60%, at least 70%, at least 80%, or at least 90%. In some embodiments, the therapeutically effective amount reduces a cell population expressing one or more of (e.g., 2, 3, 4, 5, 6 or 7 of) CD34, FLT3, CD33, CD11b, CD16, CD15, and CD66b by at least 70%, at least 75%, at least 80%, at least 85%, at least 90%, at least 95%, or at least 99%. In some embodiments, the therapeutically effective amount reduces a cell population expressing one or more of (e.g., 2, 3, 4, 5, 6 or 7 of) CD34, FLT3, CD33, CD11b, CD16, CD15, and CD66b by at least 99%, at least 98%, at least 97%, at least 96%, at least 95%.

In some embodiments, the therapeutically effective amount reduces a cell population expressing CD34 and FLT3. In some embodiments, the therapeutically effective amount reduces a cell population expressing CD34 and FLT3 by at least 25%, at least 30%, at least 40%, at least 50%, at least 60%, at least 70%, at least 80%, or at least 90%. In some embodiments, the therapeutically effective amount reduces a cell population expressing CD34 and FLT3 by at least 70%, at least 75%, at least 80%, at least 85%, at least 90%, at least 95%, or at least 99%. In some embodiments, the therapeutically effective amount reduces a cell population expressing CD34 and FLT3 by least 99%, at least 98%, at least 97%, at least 96%, at least 95%. In some embodiments, the therapeutically effective amount reduces a cell population expressing CD33 (e.g., by any of the percentages mentioned in this or preceding paragraph). In some embodiments, the therapeutically effective amount reduces a cell population expressing CD11b (e.g., by any of the percentages mentioned in this or preceding paragraph). In some embodiments, the therapeutically effective amount reduces a cell population expressing FLT3 (e.g., by any of the percentages mentioned in this or preceding paragraph). In some embodiments, the therapeutically effective amount reduces a cell population expressing CD16 (e.g., by any of the percentages mentioned in this or preceding paragraph). In some embodiments, the therapeutically effective amount reduces a cell population expressing CD15 (e.g., by any of the percentages mentioned in this or preceding paragraph). In some embodiments, the therapeutically effective amount reduces a cell population expressing CD66b (e.g., by any of the percentages mentioned in this or preceding paragraph). Any of the reductions described herein can be relative to control or baseline (e.g., relative to the level of the cells in the subject before administration of this therapy).

In some embodiments, administration to a subject of a bispecific humanized antibody or fragment described herein or a pharmaceutical composition described herein significantly reduces the number or frequency of FLT3 expressing cells. In some embodiments, administration to a subject of a bispecific humanized antibody or fragment described herein or a pharmaceutical composition described herein reduces the number or frequency of FLT3 expressing cells by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of a bispecific humanized antibody or fragment described herein or a pharmaceutical composition described herein reduces the number or frequency of FLT3 expressing cells by at least 60% relative to control or baseline (e.g., relative to the level of the cells in the subject before administration of this therapy). In some embodiments, administration to a subject of a bispecific humanized antibody or fragment described herein or a pharmaceutical composition described herein reduces the number or frequency of FLT3 expressing cells by at least 70% relative to control or baseline (e.g., relative to the level of the cells in the subject before administration of this therapy). In some embodiments, administration to a subject of a bispecific humanized antibody or fragment described herein or a pharmaceutical composition described herein reduces the number or frequency of FLT3 expressing cells by at least 80% relative to control or baseline (e.g., relative to the level of the cells in the subject before administration of this therapy). In some embodiments, administration to a subject of a bispecific humanized antibody or fragment described herein or a pharmaceutical composition described herein reduces the number or frequency of FLT3 expressing cells by at least 90% relative to control or baseline (e.g., relative to the level of the cells in the subject before administration of this therapy). In some embodiments, administration to a subject of a bispecific humanized antibody or fragment described herein or a pharmaceutical composition described herein reduces the number or frequency of FLT3 expressing cells by at least 95% relative to control or baseline (e.g., relative to the level of the cells in the subject before administration of this therapy).

In some of these embodiments, the reduction of FLT3 expressing cells is in bone marrow of the subject being treated (e.g., in bone marrow mononuclear cells). In some of these embodiments, the reduction of FLT3 expressing cells is in circulating blood cells of the subject being treated. In some of these embodiments, the reduction of FLT3 expressing cells is reduction of cancer cells in the subject being treated.

In some embodiments, the disclosure provides methods of eliminating or reducing hematopoietic stem cells (HSC) and/or hematopoietic progenitors (HP). In some embodiments, the disclosure provides methods of eliminating or reducing hematopoietic stem cells and/or progenitor cells comprising administering a bispecific humanized antibody described herein. In some embodiments, the method comprises selecting a patient in need of eliminating or reducing HSC/HP and administering to the patient a therapeutically effective amount of a pharmaceutical composition comprising a bispecific humanized antibody binding to human FLT3 expressed by HSC/HP and to human CD3 expressed by T-cells, wherein the bispecific humanized antibody redirects T-cells to eliminate HSC/HP of the patient.

In some embodiments, the methods for preparing or conditioning a subject in need thereof for hematopoietic cell transplantation are used in a subject with any cancer described herein.

In some embodiments, the methods described herein further comprise hematopoietic stem cell (HSC)/hematopoietic progenitor (HP) cell transplantation. In some embodiments, the HSC/HP transplantation includes transplantation of donor HSC/HP cells.

In some embodiments, the disclosure provides methods of hematopoietic stem cell/hematopoietic progenitor cell transplantation in a subject comprising:
(i) reducing hematopoietic stem cells (HSC) and/or hematopoietic progenitors (HP) by administration of any bispecific humanized antibody or fragment described herein or any pharmaceutical composition described herein to the subject,
(ii) transplanting donor HSCs/HP to the subject following reduction of the subject's HSC/HP cell population.

In some embodiments, the disclosure provides methods of hematopoietic stem cell/hematopoietic progenitor cell transplantation in a subject comprising:
(i) reducing hematopoietic stem cells (HSC) and/or hematopoietic progenitors (HP) by administering of an antibody described herein as 118BA #3 WT (which is also referenced herein as #3 or #3 WT), 118BA 3A (which is also referenced herein as 3A or #3A), 118BA 3B (which is also referenced herein as 3B or #3B), 118BA 3C (which is also referenced herein as 3C or #3C), 118BA 3a2 (which is also referenced herein as 3a2 or #3a2), 118BA 3a1 (which is also referenced herein as 3a1 or #3a1), 118BA 3a3 (which is also referenced herein as 3a3 or #3a3), 118BA #6 (which is also referenced herein as #6) or 118BA #5 (which is also referenced herein as #5), or any antigen binding fragment thereof, to the subject,
(ii) transplanting donor HSCs/HP to the patient following reduction of the subjects HSC/HP cell population.

In some embodiments, the disclosure provides methods of hematopoietic stem cell/hematopoietic progenitor cell transplantation in a subject comprising:
(i) reducing hematopoietic stem cells (HSC) and/or hematopoietic progenitors (HP) by administering a bispecific humanized antibody or fragment described herein to the subject,
(ii) administering a checkpoint inhibitor therapy, and
(iii) transplanting donor HSCs/HP to the patient following reduction of the subjects HSC/HP cell population.

In some embodiments, the disclosure provides methods of hematopoietic stem cell/hematopoietic progenitor cell transplantation in a subject comprising:
(i) reducing hematopoietic stem cells (HSC) and/or hematopoietic progenitors (HP) by administering a bispecific humanized antibody or fragment described herein to the subject, wherein the bispecific humanized antibody reduces the HSC/HP population by at least 90%,
(ii) administering a checkpoint inhibitor therapy, and
(iii) transplanting donor HSCs/HP to the patient following reduction of the subjects HSC/HP cell population.

### Cancers to be Treated

Cancers can be treated in accordance with the methods described herein. In some embodiments, the cancer to be treated is a hematologic cancer. Examples of hematologic cancers that are treated in accordance with the methods described herein include, but are not limited to, acute myeloid leukemia (AML), acute lymphoblastic leukemia (ALL), chronic myeloid leukemia (CML), chronic lymphocytic leukemia (CLL), Blastic plasmacytoid dendritic cell neoplasm (BPDCN), peripheral T cell lymphoma, follicular lymphoma, diffuse large B cell lymphoma, Hodgkin lymphoma, non-Hodgkin lymphoma, neuroblastoma, a non-malignant inherited or acquired marrow disorder, multiple myeloma, or a dendritic cell neoplasm. In some embodiments, the cancer is a hematologic cancer. In some embodiments, the cancer is acute myeloid leukemia (AML). In some embodiments, the cancer is acute lymphoblastic leukemia (ALL). In some embodiments, the cancer is chronic myeloid leukemia (CML). In some embodiments, the cancer is chronic lymphocytic leukemia (CLL). In some embodiments, the cancer is blastic plasmacytoid dendritic cell neoplasm (BPDCN). In some embodiments, the cancer is peripheral T cell lymphoma. In some embodiments, the cancer is follicular lymphoma. In some embodiments, the cancer is diffuse large B cell lymphoma. In some embodiments, the cancer is Hodgkin lymphoma. In some embodiments, the cancer is non-Hodgkin lymphoma. In some embodiments, the cancer is neuroblastoma. In some embodiments, the cancer is a non-malignant inherited or acquired marrow disorder. In some embodiments, the cancer is multiple myeloma. In some embodiments, the cancer is a dendritic cell neoplasm.

In some embodiments, the cancer is the result of a non-malignant inherited or acquired marrow disorder. Examples of non-malignant inherited or acquired marrow disorders that are treated in accordance with the methods described herein include, but are not limited to, sickle anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, or congenital thrombocytopenia. In some embodiments, the non-malignant inherited or acquired marrow disorder is sickle cell anemia. In some embodiments, the non-malignant inherited or acquired marrow disorder is beta-thalassemia major. In some embodiments, the non-malignant inherited or acquired marrow disorder is refractory Diamond-Blackfan anemia. In some embodiments, the non-malignant inherited or acquired marrow disorder is myelodysplastic syndrome. In some embodiments, the non-malignant inherited or acquired marrow disorder is idiopathic severe aplastic anemia. In some embodiments, the non-malignant inherited or acquired marrow disorder is paroxysmal nocturnal hemoglobinuria. In some embodiments, the non-malignant inherited or acquired marrow disorder is pure red cell aplasia. In some embodiments, the non-malignant inherited or acquired marrow disorder is Fanconi anemia. In some embodiments, the non-malignant inherited or acquired marrow disorder is amegakaryocytosis. In some embodiments, the non-malignant inherited or acquired marrow disorder is congenital thrombocytopenia.

### Methods of Administration

The bispecific humanized anti-FLT3/CD3 antibodies or fragments described herein (and pharmaceutical compositions comprising such antibodies) can be administered to a subject by any suitable means which include, but are not limited to, parenteral (e.g., intravenous, intraarterial, intramuscular, intraosseous, intracerebral, intracerebroventricular, intrathecal, subcutaneous), intraperitoneal, intratumoral, intrapulmonary, intradermal, transdermal, conjunctival, intraocular, intranasal, intratracheal, oral and local intralesional routes of administration. In some embodiments, the bispecific humanized antibodies or fragments described herein are administered intravenously, intraarterially, intraperitoneally or intratumorally.

In some embodiments, the bispecific humanized antibodies or fragments described herein are administered intravenously (e.g., by a bolus or continuous infusion). In some embodiments, the bispecific humanized antibody or fragments described herein are administered intraperitoneally. In some embodiments, the bispecific humanized antibody or fragments described herein are administered intramuscularly. In some embodiments, the bispecific humanized antibody or fragments described herein are administered subcutaneously. In some embodiments, the bispecific humanized antibody or fragments described herein are administered intratumorally (such as by an injection into the tumor of the cancer being treated). In some embodiments, the bispecific humanized antibody or fragments described herein are administered intravenously, intraperitoneally, or intratumorally.

Various dosing schedules of the bispecific humanized antibody or fragment thereof described herein (and pharmaceutical compositions comprising such antibodies) are contemplated including single administration or multiple administrations over a period of time. The methods of administration include, without limitation, bolus administration, pulse infusions, and continuous infusions.

In some embodiments, any bispecific humanized antibody or fragment thereof or pharmaceutical composition described herein is administered one, two, three, four, five, six, seven, eight, nine, ten, eleven, twelve, thirteen, fourteen, or more times. In some embodiments, any bispecific humanized antibody or fragment thereof or pharmaceutical composition described herein is administered once. In some embodiments, any bispecific humanized antibody or fragment thereof or pharmaceutical composition described herein is effective in methods described herein when administered intravenously once (e.g., without further repeat administrations).

In some embodiments, any bispecific humanized antibody or fragment thereof or pharmaceutical composition described herein is administered every about 1 to 7 days for about 1 to 8 weeks. In some embodiments, any bispecific humanized antibody or fragment thereof or pharmaceutical composition described herein is administered every about 1 to 7 days for about 1 to 4 weeks. In some embodiments, any bispecific humanized antibody or fragment thereof or pharmaceutical composition described herein is administered every about 3 to 7 days for about 2 to 3 weeks. In some embodiments, any bispecific humanized antibody or fragment thereof or pharmaceutical composition described herein is administered every about 3 days for about 2 weeks to every about 7 days for about 3 weeks. In some embodiments, any bispecific humanized antibody or fragment thereof or pharmaceutical composition described herein is administered every about 2 to 4 days for about 2 to 3 weeks (e.g., 2 weeks or 3 weeks).

In some embodiments, any bispecific humanized antibody or fragment thereof or pharmaceutical composition described herein is administered 1 day, 2 days, 3 days, 4 days, 5 days, 6 days, or 7 days a week (e.g., once a week, twice a week, every other day or every day). In some embodiments, any bispecific humanized antibody or fragment thereof or pharmaceutical composition described herein is administered for 1 week, 2 weeks, 3 weeks, 4 weeks, 5 weeks, 6 weeks, 7 weeks or 8 weeks. In some embodiments, any bispecific humanized antibody or fragment thereof or pharmaceutical composition described herein is administered for less than 6 weeks, less than 5 weeks, less than 4 weeks, less than 3 weeks or less than 2 weeks. In some embodiments, any bispecific humanized antibody or fragment thereof or pharmaceutical composition described herein is administered once in every two days or less frequently (e.g., for 1 to 3 weeks). In some embodiments, any bispecific humanized antibody or fragment thereof or pharmaceutical composition described herein is administered once in every three days or less frequently (e.g., for 1 to 3 weeks). In some embodiments, any bispecific humanized antibody or fragment thereof or pharmaceutical composition described herein is administered once in every four days or less frequently (e.g., for 1 to 3 weeks). In some embodiments, any bispecific humanized antibody or fragment thereof or pharmaceutical composition described herein is administered once in every five days or less frequently (e.g., for 1 to 3 weeks). In some embodiments, any bispecific humanized antibody or fragment thereof or pharmaceutical composition described herein is administered once a week or less frequently (e.g., for 1 to 3 weeks).

In some embodiments, the administration (of the antibodies, fragments or compositions described herein) is every 3 days for about 2 weeks. In some embodiments, the administration is every 4 days for about 2 weeks. In some embodiments, the administration is every 5 days for about 2 weeks. In some embodiments, the administration is every 7 days for about 2 weeks. In some embodiments, the administration is every 3 days for about 3 weeks. In some embodiments, the administration is every 4 days for about 3 weeks. In some embodiments, the administration is every 5 days for about 3 weeks. In some embodiments, the administration is every 7 days for about 3 weeks.

In some embodiments, the administration is once a week for 1 week, 2 weeks, 3 weeks, 4 weeks, 5 weeks, or 6 weeks. In some embodiments, the administration is twice a week for 1 week, 2 weeks, 3 weeks, 4 weeks, 5 weeks, or 6 weeks. In some embodiments, the administration is three times a week for 1 week, 2 weeks, 3 weeks, 4 weeks, 5 weeks, or 6 weeks. In some embodiments, the administration is four times a week for 1 week, 2 weeks, 3 weeks, 4 weeks, 5 weeks, or 6 weeks. In some embodiments, the administration is five times a week for 1 week, 2 weeks, 3 weeks, 4 weeks, 5 weeks, or 6 weeks. In some embodiments, the administration is six times a week for 1 week, 2 weeks, 3 weeks, 4 weeks, 5 weeks, or 6 weeks. In some embodiments, the administration is seven times a week for 1 week, 2 weeks, 3 weeks, 4 weeks, 5 weeks, or 6 weeks.

In some embodiments, the administration is once every week, once every two weeks, once every three weeks, once every four weeks, once every five weeks, or once every 6 weeks. In some embodiments, the administration is once, two, three, four, five, six, seven, eight, nine ten, eleven, twelve, thirteen, fourteen, fifteen, sixteen, seventeen, eighteen, nineteen, or twenty times (e.g., in the course of treatment).

The administrations described herein include regimens wherein the initial dose of any therapy described herein is followed by one or more lower doses, or wherein the initial dose is followed by one or more higher doses. In some embodiments, the initial dose is followed by one or more lower doses. In some embodiments, the initial dose is followed by one or more higher doses.

In some embodiments, the initial treatment period (where any therapy described herein is administered, e.g., once a month, once in two weeks, once a week, twice a week or three times a week) is followed by a withdrawal period in which the therapy is not administered (for, e.g., a week, two weeks, three weeks, four weeks, six weeks, two months, three months, four months, six months or one year), and then followed by a second treatment period (where the therapy is administered, e.g., once a month, once in two weeks, once a week, twice a week or three times a week). Such initial treatment and such second treatment periods can last, for example, two weeks, three weeks, four weeks, six weeks (where the initial treatment period can be the same or different from the second treatment period). This course of treatment (having the initial treatment period, a withdrawal period and a second treatment period) can be repeated twice, three times, four times, five times, six times, ten times or more than ten times.

In some embodiments, a therapeutically effective amount of any bispecific humanized antibody or fragment thereof or pharmaceutical composition described herein is administered to a subject or patient. A therapeutically effective amount depends on the method used, the cancer being treated, the severity of cancer being treated, the route of administration, the target site, the condition of the patient (e.g., age, body weight, health), the responsiveness of the patient, other medications used by the patient, and other factors to be considered at the discretion of the medical practitioner performing the treatment.

In some embodiments, the dosage of any bispecific humanized antibody or fragment thereof described herein is from about 0.01 mg/kg to about 10 mg/kg of the patient's body weight. In some embodiments, the dosage is from about 0.01 mg/kg to about 2 mg/kg of the patient's body weight. In some embodiments, the dosage is from about 0.05 mg/kg to about 1 mg/kg of the patient's body weight. In some embodiments, the dosage is from about 0.1 mg/kg to about 0.5 mg/kg of the patient's body weight. In some embodiments, the dosage is from about 0.1 mg/kg to about 0.3 mg/kg of the patient's body weight. In some embodiments, the dosage is about 0.01 mg/kg, about 0.1 mg/kg, about 0.5 mg/kg, about 1 mg/kg, about 1.5 mg/kg, or about 2 mg/kg of the patient's body weight. In some embodiments, the dosage is about 0.1 mg/kg of the patient's body weight. In some embodiments, the dosage is about 0.2 mg/kg of the patient's body weight. In some embodiments, the dosage is about 0.3 mg/kg of the patient's body weight.

In some embodiments, the hematopoietic cell transplantation occurs 5 days to 5 weeks after the administering of a bispecific humanized antibody or fragment, or a pharmaceutical composition comprising the same. In some embodiments, the performing of the hematopoietic cell transplantation occurs about 2 to 3 weeks after the administering of any bispecific humanized antibody or fragment, or a pharmaceutical composition comprising the same, described herein. In some embodiments, the performing of the hematopoietic cell transplantation occurs about 1 week to 4 weeks after the administering. In some embodiments, the performing of the hematopoietic cell transplantation occurs about 10 days to 25 days after the administering. In some embodiments, the performing of the hematopoietic cell transplantation occurs about 10 days to 20 days after the administering. In some embodiments, the performing of the hematopoietic cell transplantation occurs about 2 weeks after the administering. In some embodiments, the performing of the hematopoietic cell transplantation occurs about 3 weeks after the administering. In some embodiments, the performing of the hematopoietic cell transplantation occurs at least 5 days or 1 week after the administering. In some embodiments, the performing of the hematopoietic cell transplantation occurs at least 2 weeks after the administering. In some embodiments, the performing of the hematopoietic cell transplantation occurs less than 3 weeks after the administering. In some embodiments, the performing of the hematopoietic cell transplantation occurs less than 4 weeks after the administering. In some embodiments, the performing of the hematopoietic cell transplantation occurs less than 5 weeks after the administering.

### Patient Populations

In some embodiments, a patient or subject is treated with a bispecific humanized antibody or fragment thereof described herein. In some embodiments, the patient or subject is a mammal, e.g. a human, a non-human primate, a dog, a cat, a rabbit, a cow, a horse, a goat, a sheep, or a pig. In some embodiments, the subject is a human.

In some embodiments, the patient or subject being treated in accordance with the methods described herein has been diagnosed with cancer. Methods for cancer diagnosis are known in the art. In some embodiments, the cancer is early stage cancer. In some embodiments, the cancer is advanced stage cancer. In some embodiments, the cancer is a high-grade tumor. In some embodiments, the cancer is a low-grade tumor.

In some embodiments, the patient or subject being treated in accordance with the methods described herein has been diagnosed with a hematopoietic cancer. In some embodiments, the hematopoietic cancer is Acute Myeloid Leukemia (AML), Acute Lymphoblastic Leukemia (ALL), Chronic Lymphocytic Leukemia (CLL), blastic plasmacytoid dendritic cell neoplasm (BPDCN), Chronic Myeloid Leukemia (CML), peripheral T cell lymphoma, follicular lymphoma, diffuse large B cell lymphoma, Hodgkin lymphoma, non-Hodgkin lymphoma, neuroblastoma, or multiple myeloma.

In some embodiments, the patient or subject being treated in accordance with the methods described herein has been diagnosed with Acute Myeloid Leukemia (AML). In some embodiments, the patient or subject being treated in accordance with the methods described herein has been diagnosed with Acute Lymphoblastic Leukemia (ALL). In some embodiments, the patient or subject being treated in accordance with the methods described herein has been diagnosed with Chronic Lymphocytic Leukemia (CLL). In some embodiments, the patient or subject being treated in accordance with the methods described herein has been diagnosed with blastic plasmacytoid dendritic cell neoplasm (BPDCN). In some embodiments, the patient or subject being treated in accordance with the methods described herein has been diagnosed with Chronic Myeloid Leukemia (CML). In some embodiments, the patient or subject being treated in accordance with the methods described herein has been diagnosed with peripheral T cell lymphoma. In some embodiments, the patient or subject being treated in accordance with the methods described herein has been diagnosed with follicular lymphoma. In some embodiments, the patient or subject being treated in accordance with the methods described herein has been diagnosed with diffuse large B cell lymphoma. In some embodiments, the patient or subject being treated in accordance with the methods described herein has been diagnosed with Hodgkin lymphoma. In some embodiments, the patient or subject being treated in accordance with the methods described herein has been diagnosed with non-Hodgkin lymphoma. In some embodiments, the patient or subject being treated in accordance with the methods described herein has been diagnosed with neuroblastoma. In some embodiments, the patient or subject being treated in accordance with the methods described herein has been diagnosed with multiple myeloma. In some embodiments, the patient or subject being treated in accordance with the methods described herein has been diagnosed with a dendritic cell neoplasm.

In some embodiments, the patient or subject being treated in accordance with the methods described herein has been diagnosed with a non-malignant inherited acquired marrow disorder. In some embodiments, the non-malignant inherited acquired marrow disorder is sickle cell anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, congenital thrombocytopenia, or Severe Combined Immunodeficiency (SCID).

In some embodiments, the patient or subject being treated in accordance with the methods described herein has been diagnosed with sickle cell anemia. In some embodiments, the patient or subject being treated in accordance with the methods described herein has been diagnosed with beta-thalassemia major. In some embodiments, the patient or subject being treated in accordance with the methods described herein has been diagnosed with refractory Diamond-Blackfan anemia. In some embodiments, the patient or subject being treated in accordance with the methods described herein has been diagnosed with myelodysplastic syndrome. In some embodiments, the patient or subject being treated in accordance with the methods described herein has been diagnosed with idiopathic severe aplastic anemia. In some embodiments, the patient or subject being treated in accordance with the methods described herein has been diagnosed with paroxysmal nocturnal hemoglobinuria. In some embodiments, the patient or subject being treated in accordance with the methods described herein has been diagnosed with pure red cell aplasia. In some embodiments, the patient or subject being treated in accordance with the methods described herein has been diagnosed with Fanconi anemia. In some embodiments, the patient or subject being treated in accordance with the methods described herein has been diagnosed with amegakaryocytosis. In some embodiments, the patient or subject being treated in accordance with the methods described herein has been diagnosed with congenital thrombocytopenia. In some embodiments, the patient or subject being treated in accordance with the methods described herein has been diagnosed with Severe Combined Immunodeficiency (SCID).

In some embodiments, a patient or subject in need of eliminating hematopoietic stem cells (HSCs) and/or hematopoietic progenitors is treated with a bispecific humanized antibody or fragment thereof described herein. In some embodiments, the patient or subject in need of eliminating hematopoietic stem cells (HSCs) and/or hematopoietic progenitors is treated with a bispecific humanized antibody or fragment thereof described herein suffers from one or more of Acute Myeloid Leukemia (AML), Acute Lymphoblastic Leukemia (ALL), Chronic Lymphocytic Leukemia (CLL), blastic plasmacytoid dendritic cell neoplasm (BPDCN), Chronic Myeloid Leukemia (CML), peripheral T cell lymphoma, follicular lymphoma, diffuse large B cell lymphoma, Hodgkin lymphoma, non-Hodgkin lymphoma, non-hematological malignancies such as neuroblastoma, non-malignant inherited and acquired marrow disorders (e.g. sickle cell anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, or congenital thrombocytopenia), multiple myeloma, Severe Combined Immunodeficiency (SCID) and other disorders that are treated using Bone Marrow (BM)/Hematopoietic Stem Cell (HSC) transplantation.

In some embodiments, the patient or subject being treated has previously undergone one or more cancer therapies (e.g. vaccine, small molecule targeted therapy, chemotherapy, radiotherapy, or immunotherapy), and has developed resistance to one or more of the previous cancer therapies. In some embodiments, the patient or subject being treated is resistant to chemotherapy. In some embodiments, the patient or subject being treated is resistant to small molecule targeted therapy. In some embodiments, the patient or subject being treated is resistant to another immunotherapy. In some embodiments, the patient or subject being treated is resistant to a vaccine.

In some embodiments, the patient or subject has a type of cancer that is known or expected to express FLT3 on the surface of its cells. In some embodiments, the subject being treated has a type of cancer, the cells of which express one or more splice variants of FLT3.

In some embodiments, the patient or subject being treated has a cancer that has been determined, using skills known in the art, to express FLT3 on the surface of its cells that can be targeted by a bispecific humanized antibody or fragment thereof. In some embodiments, the patient or subject has a cancer that has been determined, using skills known in the art, to express an FLT3 splice variant on the surface of its cells that can be targeted by a bispecific humanized antibody or fragment thereof.

### Combination Therapies and Kits

In some embodiments, any bispecific humanized anti-FLT3/CD3 antibody or fragment thereof described herein is administered to a subject in combination with one or more anti-cancer therapies. In some embodiments, the anti-cancer therapy is a chemotherapy, radiation therapy, an immunotherapy, an antibody therapy, a small molecule therapy, or another anti-cancer therapy known in the art.

In some embodiments, a bispecific humanized antibody or fragment thereof described herein is administered to a subject in combination with chemotherapy. Examples of types of chemotherapeutic agents that can be used in the methods described herein include, without limitation, an alkylating agent, a nitrosourea agent, an antimetabolite, a topoisomerase inhibitor, an aromatase inhibitor, an antitumor antibiotic, an alkaloid derived from a plant, a hormone antagonist, a P-glycoprotein inhibitor, and a platinum complex derivative. Specific examples of chemotherapeutic drugs that can be used in the methods described herein include, without limitation, taxol, paclitaxel, nab-paclitaxel, 5-fluorouracil (5-FU), gemcitabine, daunorubicin, colchicin, mitoxantrone, tamoxifen, cyclophosphamide, mechlorethamine, busulfan, uramustine, mustargen, ifosamide, bendamustine, carmustine, lomustine, semustine, fotemustine, streptozocin, thiotepa, mitomycin, diaziquone, tetrazine, altretamine, mitozolomide, temozolomide, procarbazine, hexamethylmelamine, altretamine, hexalen, trofosfamide, estramustine, treosulfan, mannosulfan, triaziquone, carboquone, nimustine, ranimustine, azathioprine, sulfanilamide, fluoropyrimidine, thiopurine, thioguanine, mercaptopurine, cladribine, capecitabine, pemetrexed, fludarabine, hydroxyurea, nelarabine or clofarabine, cytarabine, decitabine, pralatrexate, floxuridine, thioquanine, azacitidine, cladribine, pentostatin, mercaptopurine, imatinib, dactinomycin, cerubidine, actinomycin, luteomycin, epirubicin, idarubicin, plicamycin, vincristin, vinorelbine, vinflunine, paclitaxel, docetaxel, etoposide, teniposide, periwinkle, vinca, taxane, irinotecan, topotecan, camptothecin, teniposide, pirarubicin, novobiocin, merbarone, aclarubicin, amsacrine, antiandrogen, anti-estrogen, bicalutamide, medroxyprogesterone, fluoxymesterone, diethylstilbestrol, estrace, octreotide, megestrol, raloxifene, toremifene, fulvestrant, prednisone, flutamide, leuprolide, goserelin, aminoglutethimide, testolactone, anastrozole, Jetrozole, exemestane, vorozole, formestane, fadrozole, androstene, resveratrol, myosmine, catechin, apigenin eriodictyol isoliquiritigenin, mangostin, amiodarone, azithromycin, captopril, clarithromycin, cyclosporine, piperine, quercetine, quinidine, quinine, reserpine, ritonavir, tariquidar, verapamil, cisplatin, carboplatin, oxaliplatin, transplatin, nedaplatin, satraplatin, triplatin and carboplatin.

In some embodiments, the antitumor agent is selected from but not limited to suitable anti-neoplastic agents that are known to those skilled in the art and include anthracyclines (e.g. daunomycin and doxorubicin), auristatin, methotrexate (MTX), vindesine, neocarzinostatin, cis-platinum, chlorambucil, cytosine arabinoside, 5-fluorouridine, melphalan, ricin and calicheamicin including combination chemotherapy such with doxorubicin, bleomycin, vinblastine, and dacarbazine (ABVD), BEACOPP or escalated BEACOPP (bleomycin, etoposide, doxorubicin, cyclophosphamide, vincristine, procarbazine, and prednisone) and Stanford V (doxorubicin, vinblastine, mechlorethamine, vincristine, bleomycin, etoposide, and prednisone). The antitumor agent can also be immunotherapy (e.g. anti-CD20 antibody rituximab), immunotoxins (e.g. Brentuximab vedotin (SGN-35) is an immunotoxin comprised of a CD-30 directed antibody linked to the antitubulin agent monomethyl auristatin E (MMAE)), adoptive immunotherapy (cytotoxic T lymphocytes), programmed death 1 (PD-1) blockade (eg, nivolumab, pembrolizumab).

In some embodiments, any bispecific humanized antibody or fragment described herein, or any pharmaceutical composition described herein, is administered to a subject with a cancer in combination with the chemotherapy drug(s) indicated for said cancer, which chemotherapy drug(s) can be optionally administered in the dosage and/or regime of administration indicated for said cancer (e.g., AML or ALL).

In some embodiments, any bispecific humanized antibody or fragment described herein or any pharmaceutical composition described herein is administered to a subject in combination with immunotherapy. In some embodiments, the immunotherapy comprises administering a checkpoint inhibitor. In some embodiments, the checkpoint inhibitor is an anti-PD1 antagonist, an anti-PD-L1 antagonist, and an anti-CTLA4 antagonist. In some embodiments, the checkpoint inhibitor is an anti-PD1 antagonist. In some embodiments, the checkpoint inhibitor is an anti-PD-1 antibody (such as an antagonistic anti-PD-1 antibody). In some embodiments, the checkpoint inhibitor is an anti-PD-L1 antagonist. In some embodiments, the checkpoint inhibitor is an anti-PD-L1 antibody (such as an antagonistic anti-PD-L1 antibody). In some embodiments, the checkpoint inhibitor is an anti-CTLA4 antagonist (e.g., an antagonistic anti-CTLA4 antibody). In some embodiments, the checkpoint inhibitor is a Lag3 antagonist. In some embodiments, the checkpoint inhibitor is Tim3 antagonist. In some embodiments, the checkpoint inhibitor is a TIGIT antagonist. In some embodiments, the checkpoint inhibitor is an OX40 antagonist.

In some embodiments, the anti-PD1 antagonist is selected from, but not limited to, nivolumab, pembrolizumab, PDR001, Pembrolimumab (Bio X Cell), Bio X Cell Clone J116 (Cat. #BE0188), cemiplimab, and pidilizumab. In some embodiments, the anti-PD-L1 antagonist is selected from, but not limited to, atezolizumab, avelumab, durvalumab, YW243.55.S70, MPDL3280A, MDX-1105, and BMS-936559 In some embodiments, the anti-CTLA4 antagonist is selected from, but not limited to ipilimumab and tremelimumab.

In some embodiments, the any of the therapies described herein is administered to a subject in combination with radiation therapy (e.g., x-ray, gamma ray, electron beams).

In some embodiments, the checkpoint inhibitor is administered prior to administration of any humanized bispecific antibody or fragment described herein. In some embodiments, the checkpoint inhibitor is administered concomitantly with any humanized bispecific antibody or fragment described herein. In some embodiments, the checkpoint inhibitor is administered after administration of any bispecific humanized antibody or fragment described herein.

In some embodiments, a bispecific humanized antibody or fragment thereof described herein is administered to a subject before, during, or after a second therapy.

In some embodiments, the subject being treated in accordance with the methods described herein has not received an anti-cancer therapy prior to the administration of a bispecific humanized antibody or fragment thereof described herein. In some embodiments, a bispecific humanized antibody or fragment thereof described herein is administered to a subject that has received an anti-cancer therapy prior to administration of the antibody or fragment. In some, embodiments, a bispecific humanized antibody or fragment thereof described herein is administered to a subject recovering from or receiving an immunosuppressive therapy.

In some embodiments, the subject being treated in accordance with the methods described herein has not received an anti-cancer therapy prior to the administration of any bispecific humanized antibody or fragment described herein or any pharmaceutical composition described herein. In some embodiments, any bispecific humanized antibody or fragment described herein or any pharmaceutical composition described herein is administered to a subject that has received an anti-cancer therapy prior to administration of the antibody or fragment. In some, embodiments, any bispecific humanized antibody or fragment described herein or any pharmaceutical composition described herein is administered to a subject recovering from or receiving an immunosuppressive therapy.

In some embodiments, provided herein are kits comprising a bispecific humanized antibody or fragment thereof, and one or more additional cancer agents. In some embodiments, provided herein are kits comprising (i) a bispecific humanized antibody or a fragment thereof (e.g., in a therapeutically effective amount), and (ii) one or more chemotherapeutic drugs in a therapeutically effective amount, which may be less than the therapeutic amount of the drug or drugs when used without a bispecific humanized antibody.

In some embodiments, provided herein are kits comprising a bispecific humanized antibody or fragment thereof, and one or more additional checkpoint inhibitors. In some embodiments, provided herein are kits comprising (i) a bispecific humanized antibody or a fragment thereof (e.g., in a therapeutically effective amount), and (ii) one or more checkpoint inhibitors in a therapeutically effective amount, which may be less than the therapeutic amount of the drug or drugs when used without a bispecific humanized antibody. In some embodiments, provided herein are kits comprising (i) any bispecific humanized antibody or fragment described herein or any pharmaceutical composition described herein, and (ii) one or more anti-PD1 antibody, anti-PD-L1 antibody or anti-CTLA4 antibody (e.g., in a therapeutically effective amount, which may be less than the therapeutic amount of the drug or drugs when used without the antibody or fragment).

In some embodiments, kits comprising any bispecific humanized anti-FLT3/CD3 antibodies or fragments and one or more additional anti-cancer agents described herein are also contemplated.

The following examples are offered by way of illustration and not by way of limitation. Various other embodiments of the invention may be practiced, given the general description provided herein.

### EXAMPLES

### Example 1: Evaluation of Binding Properties of Three Chimeric Monoclonal Anti-FLT3 Antibodies 118BA, 1B11E7 and 281A

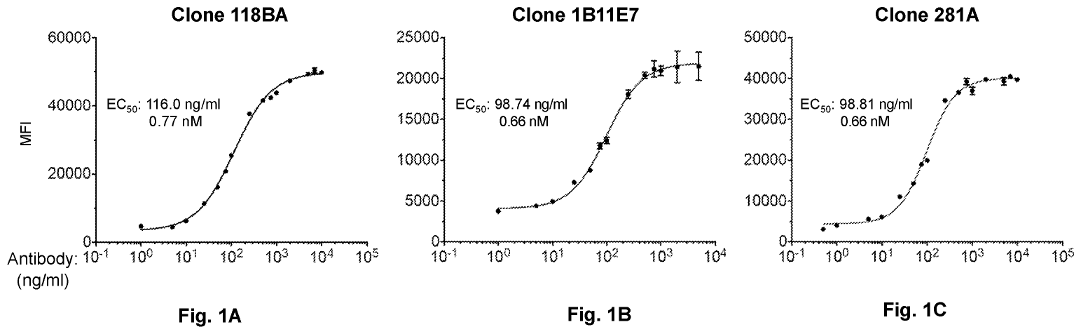

REH cells that express high levels of FLT3 were used to test the binding affinities of anti-FLT3 chimeric monoclonal antibodies to FLT3 (having mouse heavy and light chain variable regions and human constant domains). REH cells were stained with various concentrations of chimeric monoclonal antibodies prepared in PBS+2% BCS+2 mM EDTA (flow buffer). Cells were washed five times with flow buffer and stained with anti-human IgG Fc antibody conjugated to Fluorescein (Jackson Immunoresearch Laboratories, 109-095-008). Cells were stained with 7-AAD (7-AAD Viability Staining Solution, Biolegend 420404) followed by flow cytometry analysis. The binding EC50s were determined to be 0.77 nM, 0.66 nM and 0.66 nM for 118BA (disclosed in US20190389955 as having VL of SEQ ID NO: 25 and VH of SEQ ID NO:27, with US20190389955 incorporated by reference herein in its entirety), 1B11E7 (disclosed in US20190389955 as having a VL of SEQ ID NO: 21 and VH of SEQ ID NO:23) and 281A (disclosed in US20190389955 as having a VL of SEQ ID NO: 17 and VH of SEQ ID NO:19) respectively as shown in FIGS. 1A-1C.

### Example 2: Evaluation of FLT3 Ligand (FLT3L) Competition for Binding FLT3 with Three Chimeric Monoclonal Anti-FLT3 Antibodies 118BA, 1B11E7 and 281A

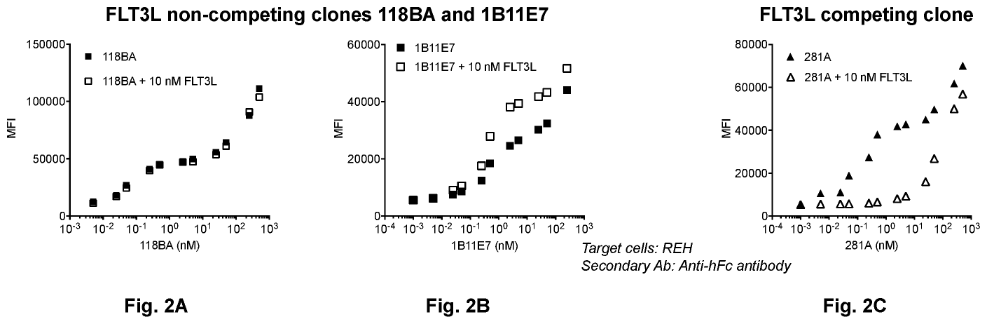

Prior to humanization, the chimeric anti-FLT3 antibodies were evaluated for competitive binding with FLT3 ligand (FLT3L). REH cells were incubated with 10 nM of recombinant human FLT3L (R&Dsystems) for 20 minutes and washed with PBS+2% BCS+2 mM EDTA (flow buffer). Cells were then stained with various concentrations of chimeric monoclonal antibodies prepared in flow buffer. Cells were washed five times with flow buffer and stained with anti-human IgG Fc antibody conjugated to Alexa Fluor 488 (Jackson Immunoresearch Laboratories, 109-545-008). Cells were stained with 7-AAD (7-AAD Viability Staining Solution, Biolegend 420404) followed by flow cytometry analysis. The binding of the chimeric antibody 118BA (disclosed in US20190389955 as having VL of SEQ ID NO: 25 and VH of SEQ ID NO:27) and 1B11E7 (disclosed in US20190389955 as having VL of SEQ ID NO: 21 and VH of SEQ ID NO.23) to REH were not affected by pretreatment of cells with FLT3L whereas 281A (disclosed in US20190389955 as having VL of SEQ ID NO: 17 and VH of SEQ ID NO:19) binding was significantly reduced with FLT3L pre-treatment as shown in FIGS. 2A-2C. This suggests that 281A antibody compete with FLT3L for binding to FLT3, but 118BA and 1B11E7 antibodies do not compete with FLT3L for binding to FLT3.

### Example 3: Evaluation of Binding Properties of Humanized Variants of Anti-FLT3 Monoclonal Antibody Clones 118BA (Variants 1 and 5), 1B11E7 (Variants 7 and 10) and 281A (Variants 1 and 5

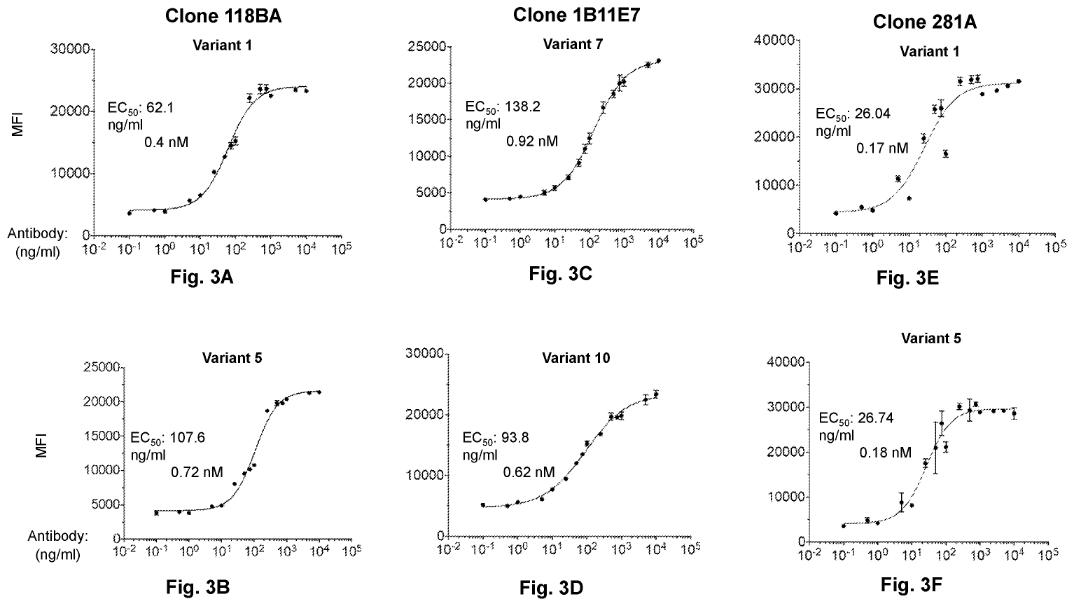

118BA variant 1 is also called 18BA-v1, and its VL and VH sequences are provided as SEQ ID NO:1 and SEQ ID NO:2, respectively. 118BA variant 5 is also called 18BA-v5, and its VL and VH sequences are provided as SEQ ID NO:88 and SEQ ID NO:89, respectively. VL and VH sequences of 1B11E7 (variants 7 and 10) and 281A (variants 1 and 5) are also provided herein (see the Sequence Listing). REH cells were stained with various concentrations of humanized 118BA variants 1 and 5, humanized 1B11E7 variants 7 and 10 and humanized 281A variants 1 and 5 prepared in PBS+2% BCS+2 mM EDTA (flow buffer). Cells were washed five times with flow buffer and stained with anti-human IgG Fc antibody conjugated to Alexa Fluor 488 (Jackson Immunoresearch Laboratories, 109-545-008). Cells were stained with 7-AAD (7-AAD Viability Staining Solution, Biolegend 420404) followed by flow cytometry analysis. The EC50 of binding was determined to be 0.4 nM, 0.72 nM, 0.92 nM, 0.62 nM, 0.17 nM and 0.18 nM for humanized 118BA variants 1 and 5, 1B11E7 variants 7 and 10 and 281A variants 1 and 5 respectively (FIGS. 3A-3F). The antibodies used are as described in Example 1.

### Example 4: Assessment of Cross-Reactivity of Humanized Anti-FLT3 Antibody Clones with Rhesus FLT3

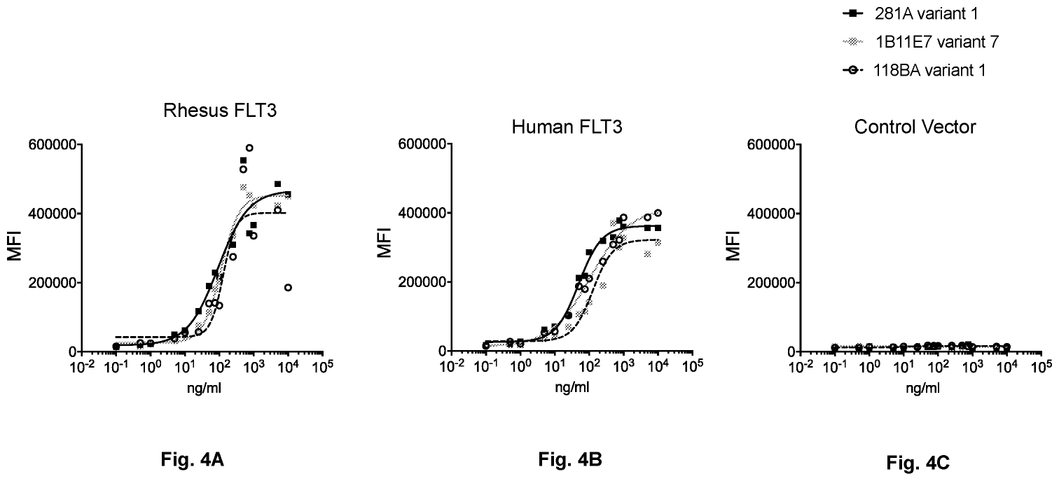

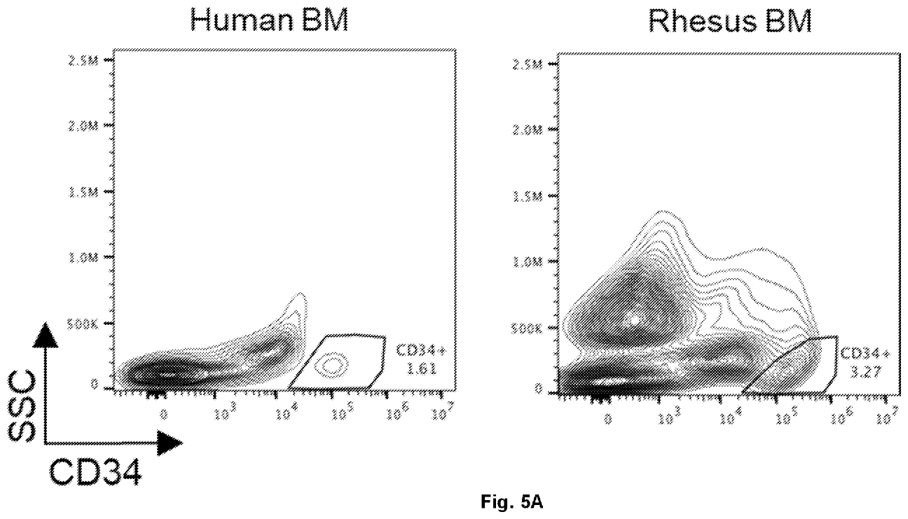

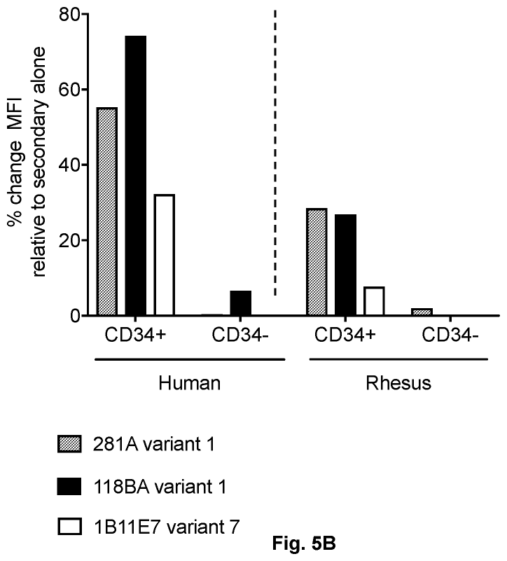

HEK293T cells were transfected with human FLT3 or rhesus FLT3 encoding plasmid DNA or control mock transfected. 24 hours post transfection, cells were stained with various concentrations of humanized anti-FLT3 antibodies 281A variant 1, 1B11E7 variant 7 and 118BA variant 1 prepared in PBS+2% BCS+2 mM EDTA (flow buffer). Cells were washed five times with flow buffer and stained with anti-human IgG APC secondary antibody (Jackson Immunoresearch Laboratories). Cells were stained with 7-AAD (7-AAD Viability Staining Solution, Biolegend 420404) followed by flow cytometry analysis. EC50 for binding rhesus FLT3 expressing cells and human FLT3 expressing cells were very similar suggesting that the FLT3 epitopes of the antibodies are conserved between human and rhesus as shown in FIGS. 4A-4B. Besides, the antibody clones did not bind the mock transfected HEK293T cells suggesting FLT3 specific binding of the antibodies (FIG. 4C).

FLT3 is normally expressed in CD34+ hematopoietic stem progenitor cells (HSPCs) and more differentiated hematopoietic progenitors (HPs). To test if the humanized antibodies bind to FLT3 expressed on CD34+ human and rhesus bone marrow HSPCs and HPs, mononuclear cells from rhesus and human bone marrow were incubated with 1 g/mL of humanized anti-FLT3 antibodies 281A variant 1, 1B11E7 variant 7, 118BA variant 1 prepared in PBS+2% BCS+2 mM EDTA buffer or buffer only and washed 3 times with buffer. Cells were incubated with PE conjugated CD34 antibody (StemCell Technologies) specific for human and rhesus CD34 prepared in buffer at 1:20 dilution and anti-human IgG APC secondary antibody (Jackson Immunoresearch Laboratories) prepared in buffer at 1:200 dilution. Cells were washed once and stained with 7-AAD (7-AAD Viability Staining Solution, Biolegend 420404) and analyzed by flow cytometry. The percent change in mean florescence intensities from APC channel relative to secondary antibody alone were determined for CD34+ and CD34− cell populations for all conditions tested. CD34+ cells showed significantly higher percent increase in MFIs in the presence of all three variants compared to CD34− cells that showed minimal percent change in MFIs. 281A variant showed the least percent increase in MFI for both human and rhesus CD34+ cells among the three variants tested. 118BA and 1B11E7 variants had similar percent increase in MFI for both human and rhesus CD34+ cells (FIGS. 5A and 5B). These results suggest that all three humanized antibodies bind to FLT3 expressed on CD34+ bone marrow HSPCs and HPs in both species and 118BA and 1B11E7 variants have superior binding property to CD34+ cells compared to the 281A variant.

### Example 5: Evaluation of Binding Properties of Chimeric and Humanized Monoclonal Anti-CD3 IgG SP34 Clone to CD3

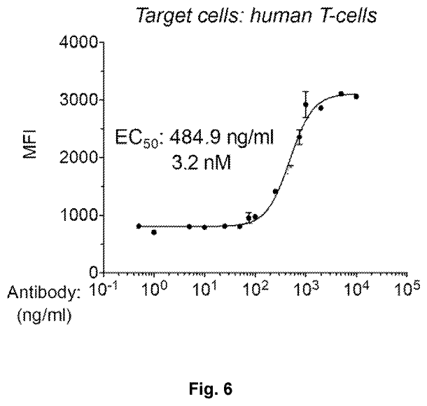

Primary human pan T cells isolated from cord blood (Source: New York Blood Center) that express high levels of CD3 were used to test the binding affinities of a chimeric monoclonal SP34 antibody to CD3 on the surface of T cells (comprising mouse heavy and light chain variable regions and human constant domains). Pan T cells were stained with various concentrations of chimeric monoclonal antibodies prepared in PBS+2% BCS+2 mM EDTA (flow buffer). Cells were washed five times with flow buffer and stained with anti-human IgG Fc antibody conjugated to Alexa Flour 488 (Jackson Immunoresearch Laboratories, 109-095-008). Cells were stained with 7-AAD (7-AAD Viability Staining Solution, Biolegend 420404) followed by flow cytometry analysis. The binding EC50s was determined 3.2 nM to be as shown in FIG. 6.

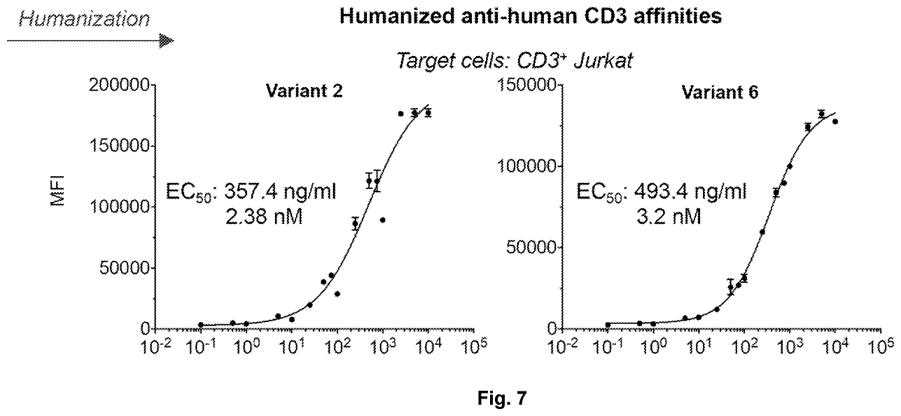

The mouse heavy and light chain variable regions of SP34 clone were humanized and two humanized variants were tested for their binding affinities to CD3 in Jurkat cells (ATCC) expressing high levels of CD3. Jurkat cells were stained with various concentrations of humanized SP34 variants 2 and 6 (variant 2 VH and VL are SEQ ID NOs: 67 and 68, respectively; variant 6 VH and VL are SEQ ID NOs: 69 and 70, respectively) antibodies prepared in PBS+2% BCS+2 mM EDTA (flow buffer). Cells were washed five times with flow buffer and stained with anti-human IgG Fc antibody conjugated to Alexa Flour 488 (Jackson Immunoresearch Laboratories, 109-095-008). Cells were stained with 7-AAD (7-AAD Viability Staining Solution, Biolegend 420404) followed by flow cytometry analysis. The binding EC50s was determined to be 2.38 nM and 3.2 nM for 118BA, variants 2 and 6 respectively as shown in FIG. 7.

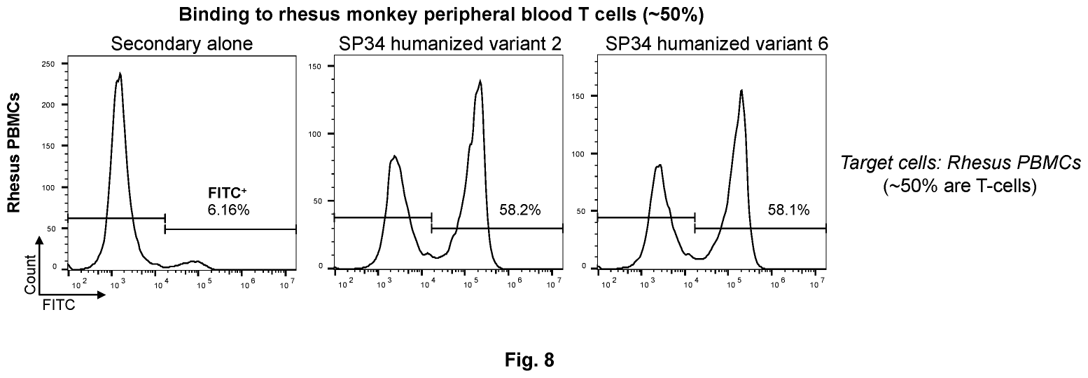

Peripheral blood contains around 45-70% T cells that express high levels of CD3. To test if the humanized antibodies cross-react with rhesus, CD3 mononuclear cells from rhesus peripheral blood were incubated with 1 μg/ml of humanized anti-CD3 SP34 variants 2 and 6 prepared in PBS+2% BCS+2 mM EDTA buffer or buffer only and washed 3 times with buffer. Cells were incubated with anti-human IgG Fc antibody conjugated to Alexa Flour 488 (Jackson Immunoresearch Laboratories, 109-095-008). Cells were stained with 7-AAD (7-AAD Viability Staining Solution, Biolegend 420404) followed by flow cytometry analysis. Both humanized variants showed binding to ˜58% of total mononuclear cells as shown in FIG. 8 suggesting binding to rhesus CD3 on T cells that generally constitutes around 45-70% of total mononuclear cells in peripheral blood.

### Example 6: Evaluation of Binding Properties of CD3×FLT3 Bispecific Antibodies 281A #1, 1B11E7 #2, 118BA #3 and 118BA Variant #4 to FLT3 in REH Cells and CD3 in Jurkat Cells

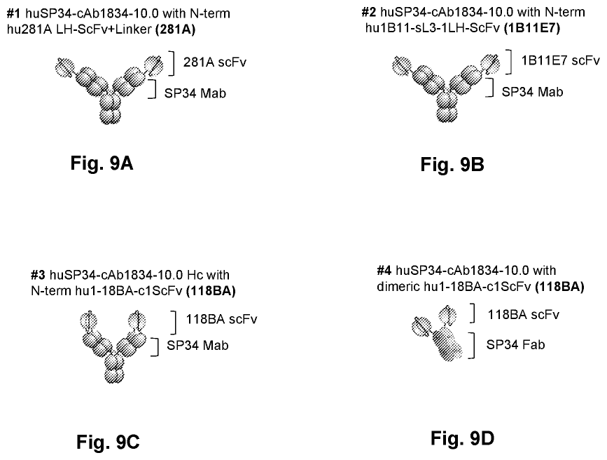

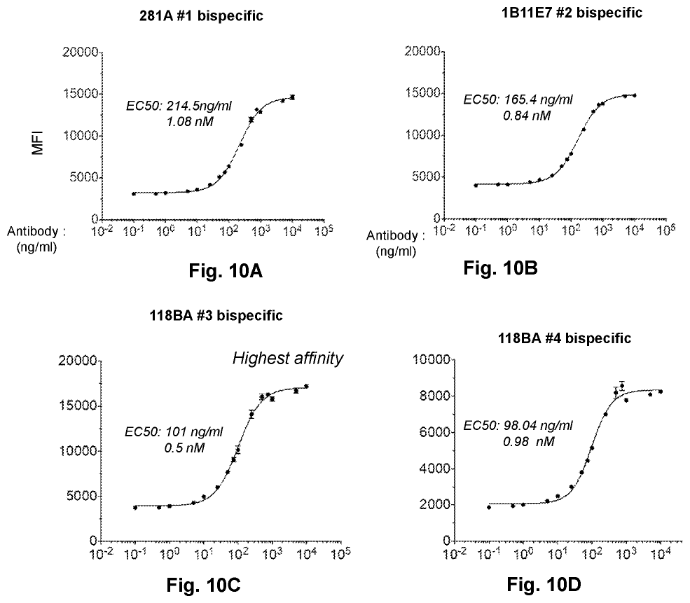

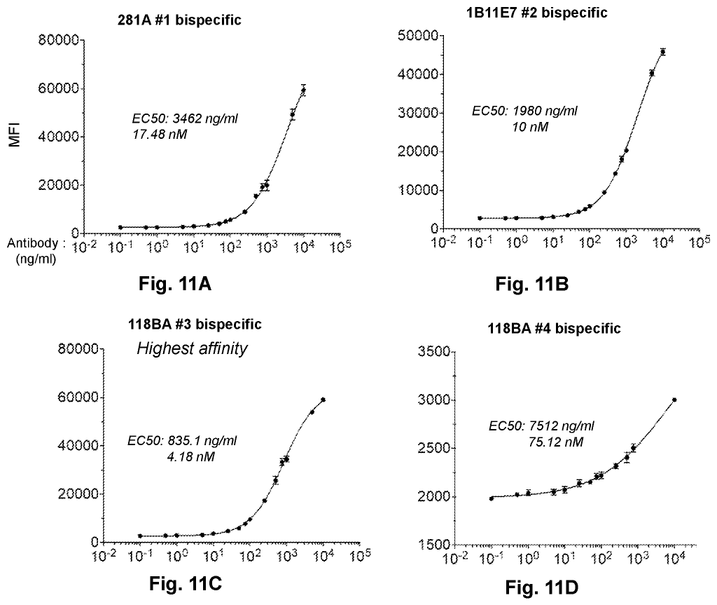

FLT3+ REH cells and CD3+ Jurkat cells were stained with various concentrations of 281A #1, 1B11E7 #2, 118BA #3 (comprising SEQ ID NOs: 8 and 53) and 118BA #4 (FIGS. 9A-9D) bispecific antibodies prepared in PBS+2% BCS+2 mM EDTA (flow buffer). Cells were washed five times with flow buffer and stained with anti-human IgG lambda antibody conjugated to FITC (Southern Biotech 2070-02). Cells were stained with 7-AAD (7-AAD Viability Staining Solution, Biolegend 420404) followed by flow cytometry analysis. The EC50s of binding to REH cells were determined to be 1.08 nM, 0.84 nM, 0.5 nM and 0.98 nM for 281A #1, 1B11E7 #2, 118BA #3, and 118BA #4 bispecific antibodies respectively as shown in FIGS. 10A-10D. The EC50s of binding to Jurkat cells were determined to be 17.48 nM, 10 nM, 4.18 nM and 75.12 nM for 281A #1, 1B11E7 #2, 118BA #3 and 118BA #4 bispecific antibodies respectively as shown in FIGS. 11A-D.

### Example 7: In Vitro Cytotoxicity and T Cell Activation of CD3×FLT3 Bispecific Antibodies 281A #1, 1B11E7 #2, 118BA #3 and 118BA #4 Towards FLT3+ REH Cells

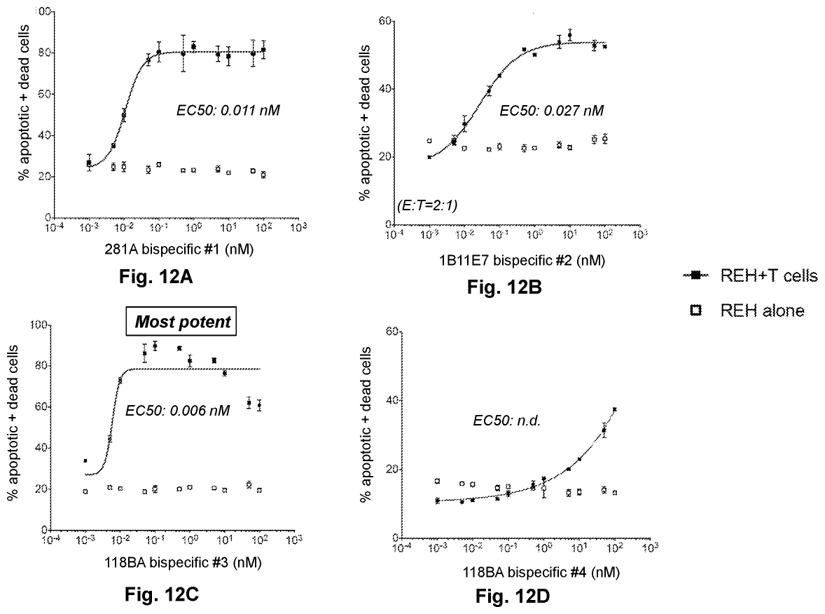

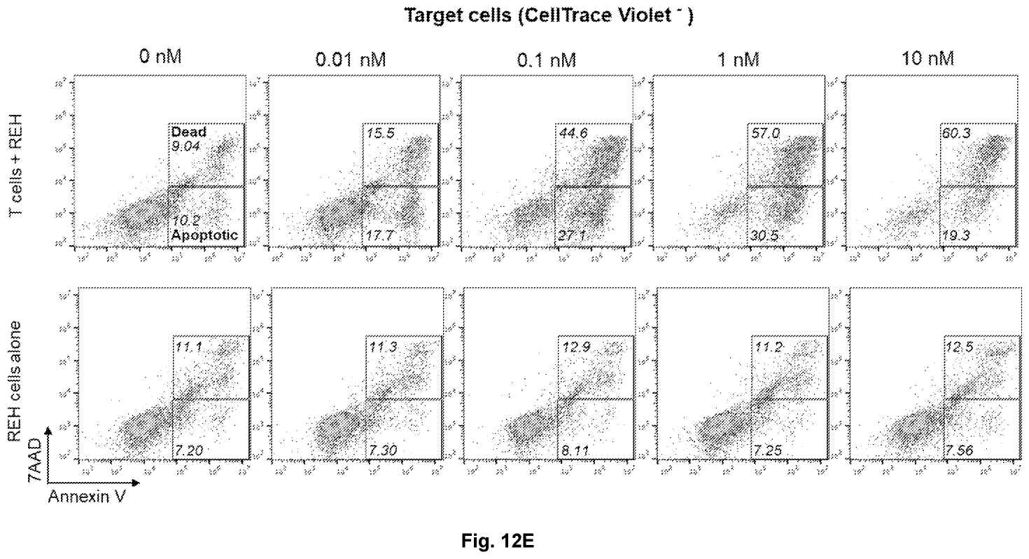

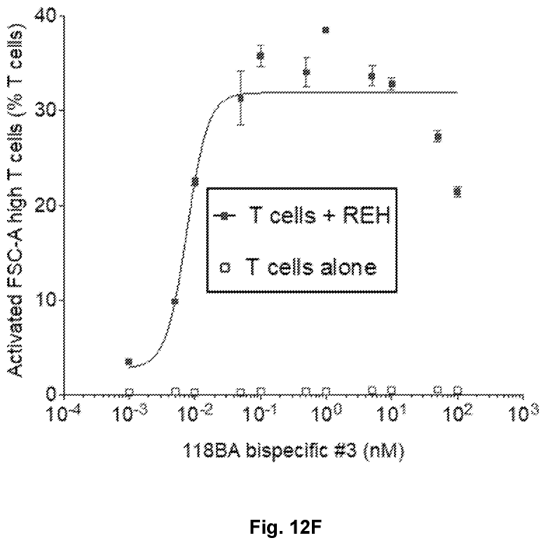

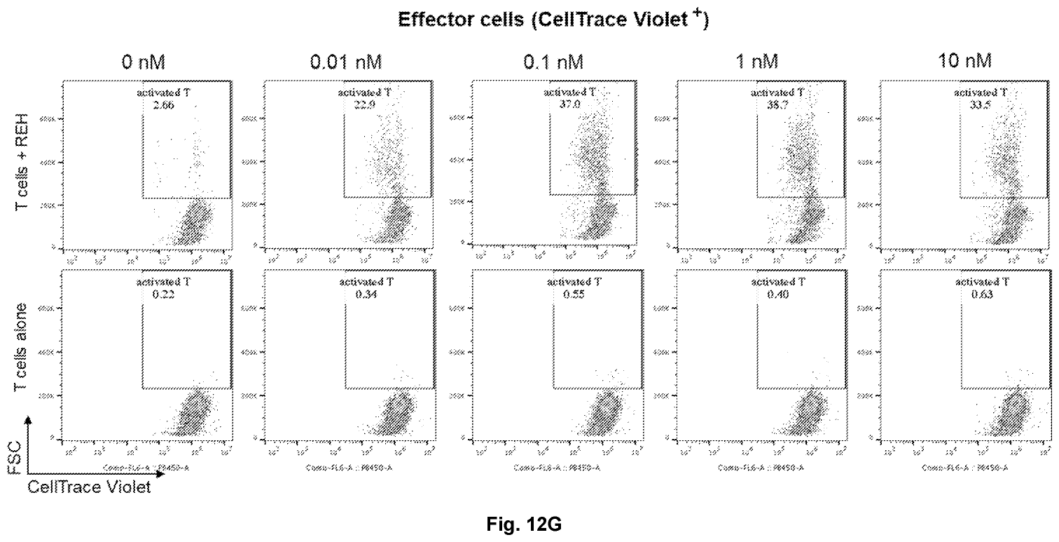

T cells from cord blood (source: New York Blood Center) were isolated using T cell isolation kit (Stemcell Technologies) and labeled with CellTrace Violet (ThermoFisher). T cells were co-cultured with FLT3 expressing REH cells (acute lymphocytic leukemia cell line) at 3:1 effector to target (E:T) ratio in the presence of 281A #1, 1B11E7 #2, 118BA #3 and 118BA #4 bispecific antibodies at various concentrations ranging from 0 nM to 100 nM. REH and T cells were also cultured separately under all conditions. Recombinant human IL2 (50 ng/ml) was added to promote T cell survival. After 3 days, cells were stained with FITC conjugated AnnexinV, an apoptosis marker and 7-AAD (7-AAD Viability Staining Solution, Biolegend 420404), a viability dye, and analyzed by flow cytometry. Cytotoxicity was measured as % target REH cells (CellTrace Violet negative) that were dead or apoptotic for each condition. The EC50 of cytotoxicity against REH cells were determined as 0.011 nM, 0.027 nM, 0.006 nM for 281A #1, 1B11E7 #2 and 118BA #3 respectively as shown in FIGS. 12A-12G. 118BA #4 antibody had significantly less cytotoxicity against REH and therefore the range of concentration tested for the antibody was not enough to calculate the EC50 of cytotoxicity. 118BA #3 antibody was found to be the most potent among the four bispecific antibodies tested. No toxicities were observed in REH cells when they were cultured with bispecific antibodies in the absence of T cells. Activation of T cells by 118BA #3 bispecific antibody was determined by measuring % activated T cells identified as CellTrace Violet positive and FSC-A high for each condition as shown in FIGS. 12E-12G. T cells were significantly activated by 118BA #3 bispecific antibody in a dose dependent manner only in the presence of target cells.

### Example 8: Binding Affinity of CD3×FLT3 118BA #3 Bispecific Antibodies in Various FLT3+ Leukemia Cell Lines

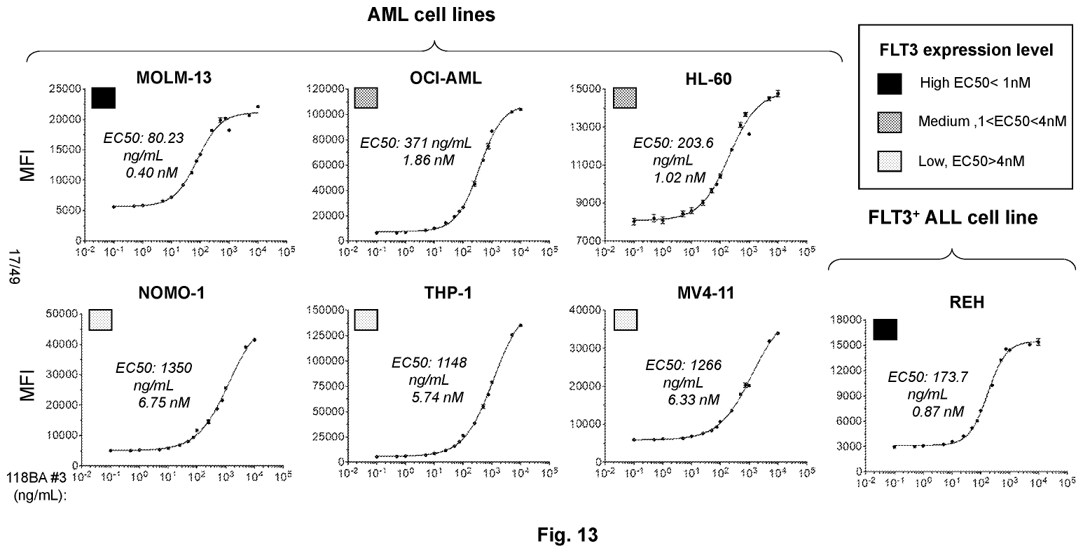

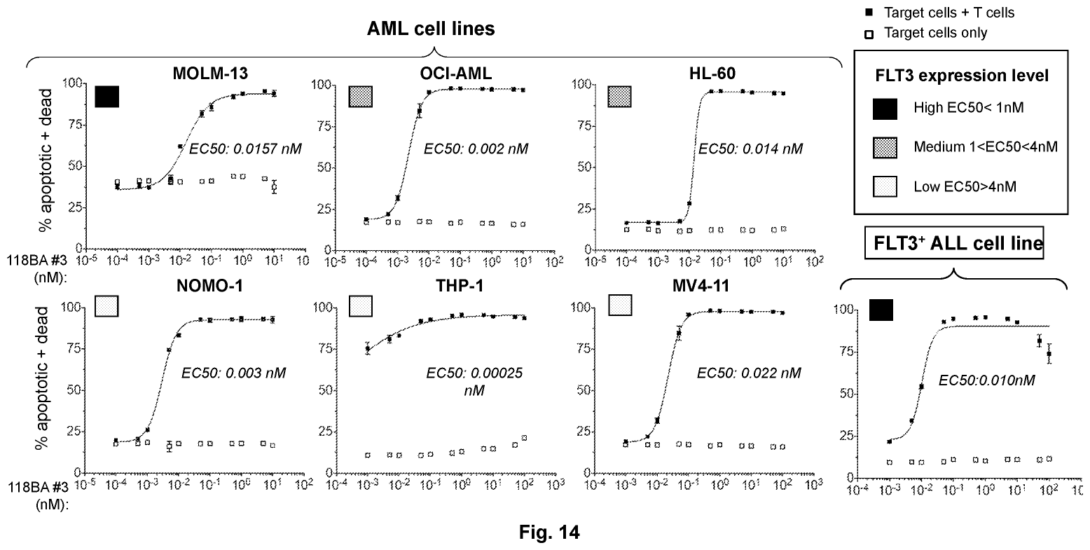

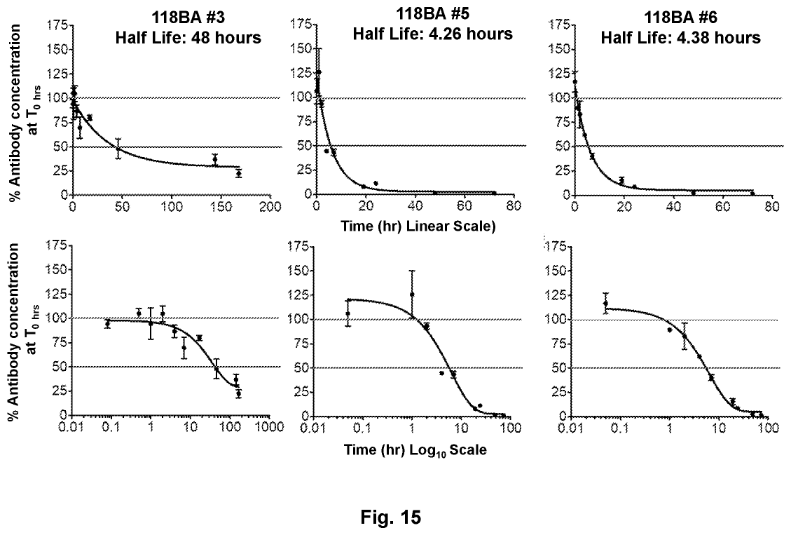

MOLM-13, OCI-AML, HL-60, NOMO-1, THP-1, MV4-11 and REH cells were stained with various concentrations of 118BA #3 bispecific antibody prepared in PBS+2% BCS+2 mM EDTA (flow buffer). Cells were washed five times with flow buffer and stained with anti-human IgG lambda antibody conjugated to FITC (Southern Biotech 2070-02). Cells were stained with 7-AAD (7-AAD Viability Staining Solution, Biolegend 420404) followed by flow cytometry analysis. The EC50s of 118BA #3 bispecific binding to MOLM-13, OCI-AML, HL-60, NOMO-1, THP-1, MV4-11 and REH were determined to be 0.40 nM, 1.86 nM, 1.02 nM, 6.75 nM, 5.74 nM, 6.33 nM and 0.87 nM respectively as shown in FIG. 13. Binding of bispecific antibody to cells should directly correlate with FLT3 expression. Therefore, based on their binding profiles, cells were categorized into high (EC50<1 nM), medium (1<EC50<4 nM) and low (EC50>4 nM) FLT3 expression levels.

### Example 9: In Vitro Cytotoxicity of CD3×FLT3 Bispecific Antibodies Towards Various Leukemia Cell Lines

T cells from cord blood (source: New York Blood Center) were isolated using T cell isolation kit (Stemcell Technologies) and labeled with CellTrace Violet (ThermoFisher). T cells were co-cultured with FLT3 expressing target MOLM-13, OCI-AML, HL-60, NOMO-1, THP-1, MV4-11 or REH cells at 3:1 effector to target (E:T) ratio in the presence of 118BA #3 bispecific antibodies at various concentrations ranging from 0 nM to 100 nM. FLT3+ target cells and T cells were also cultured separately under all conditions. Recombinant human IL2 (50 ng/ml) was added to promote T cell survival. After 3 days, cells were stained with FITC conjugated AnnexinV, an apoptosis marker and 7-AAD, a viability dye, and analyzed by flow cytometry. Cytotoxicity was measured as % target cells (CellTrace Violet negative) that were dead or apoptotic for each condition. The EC50s of cytotoxicity against MOLM-13, OCI-AML, HL-60, NOMO-1, THP-1, MV4-11 and REH cells were determined as 0.0157 nM, 0.002 nM, 0.014 nM, 0.003 nM, 0.00025 nM, 0.022 nM and 0.01 nM respectively as shown in FIG. 14. These results show that 118BA #3 bispecific antibody is cytotoxic towards a wide variety of cell lines derived from leukemia blasts. 118BA #3 antibody mediated killing of target cells did not always correlate with the binding affinities of the antibody to the target cells tested suggesting that other factors are likely to influence the killing potential of the antibodies towards specific target cells. No toxicities were observed in target cells when they were cultured with bispecific antibodies in the absence of T cells.

### Example 10: Measurement of the Half-Lives of Bispecific Antibodies in Plasma of the Peripheral Blood of C57BL/6 Mice

6-8 week old C57BL/6 mice from Taconic were injected intravenously with 25 μg of bispecific antibodies 118BA #3 (the sequence of which is provided herein and referenced above) and FcRnKO variants 118BA #5 (comprising SEQ ID NOs: 30 and 28) and 118BA #6 (comprising SEQ ID NOs: 25 and 28) antibodies. Blood was collected from mice by submandibular bleeding at various time points between 0-72 hrs for 118BA #5 and 118BA #6 and 0-168 hrs for 118BA #3. Antibody concentration in plasma of mice was determined by AlphaScreen (Amplified Luminescent Proximity Homogeneous Assay). Plasma was incubated with recombinant His-tagged human Flt-3/Flk-2 Fc chimera protein and an anti-CHI llama antibody-biotin conjugate (Thermo Scientific 7103202100) to allow biotin conjugate-bispecific antibody-FLT3 complex formation. AlphaScreen anti-His acceptor beads were then added that interact with the protein complex by binding to the recombinant His-tagged FLT3. AlphaScreen streptavidin conjugated donor beads that bind to the biotin in the complex were then added to the solution. Finally, a plate reader was used to detect the emission (520-620 nm) signals upon excitation at 680 nm. Since the presence of bispecific antibody brings the acceptor and donor beads into close proximity excitation of the donor beads causes emission from the acceptor beads and the signals are directly proportional to the concentration of bispecific antibody. Known concentrations of bispecific antibodies were diluted in control mouse plasma and used as standards in the assay. Half-lives of the antibodies were determined by measuring the time it takes to reach 50% of the antibody concentration observed in peripheral blood at 0 hr (3 mins after antibody injection) timepoint. The half-lives for 118BA #3, 118BA #5 and 118BA #6 in C57BL/6 mice are 48 hours, 4.26 hours and 4.38 hours respectively as shown in FIG. 15.

### Example 11: Evaluation of Binding Properties of 118BA #3, 118BA #3A, 118BA #3B and 118BA #3C Bispecific Antibodies

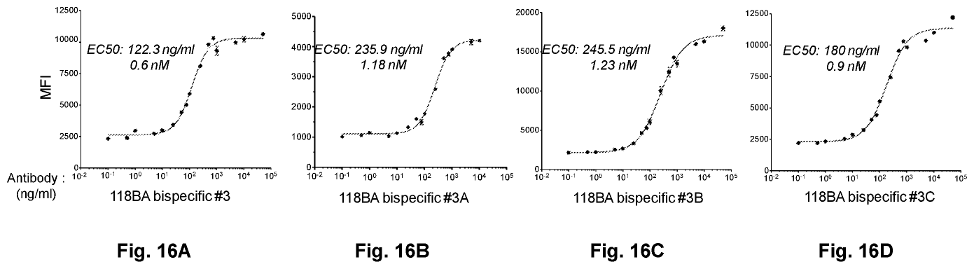

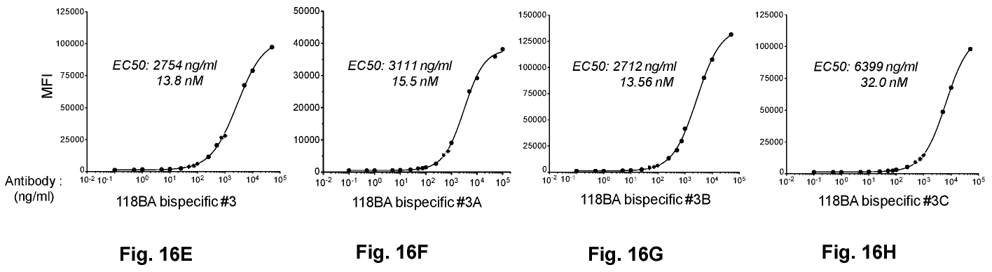

FLT3+ REH cells and CD3+ human T cells were stained with various concentrations of 118BA #3 (comprising SEQ ID NOs: 8 and 53), and its three variants 118BA #3A (comprising SEQ ID NOs: 8 and 13), 118BA #3B (comprising SEQ ID NOs: 15 and 13), and 118BA #3C (comprising SEQ ID NOs: 17 and 13), bispecific antibodies prepared in PBS+2% BCS+2 mM EDTA (flow buffer). Cells were washed five times with flow buffer and stained with anti-human IgG lambda antibody conjugated to FITC (Southern Biotech 2070-02). Cells were stained with 7-AAD (7-AAD Viability Staining Solution, Biolegend 420404) followed by flow cytometry analysis. The EC50 of binding to REH cells was determined to be 0.6 nM, 1.18 nM, 1.23 nM and 0.9 nM for 118BA #3, 118BA #3A, 118BA #3B and 118BA #3C bispecific antibodies respectively as shown in FIGS. 16A-16D. The EC50 of binding to T cells was determined to be 13.8 nM, 15.5 nM, 13.56 and 32.0 nM for 118BA #3, 118BA #3A, 118BA #3B and 118BA #3C bispecific antibodies respectively as shown in FIGS. 16E-16H.

### Example 12: In Vitro Cytotoxicity and T Cell Activation of CD3×FLT3 Bispecific Antibodies 118BA #3, 118BA #3A, 118BA #3B and 118BA #3C Towards FLT3+ REH Cells

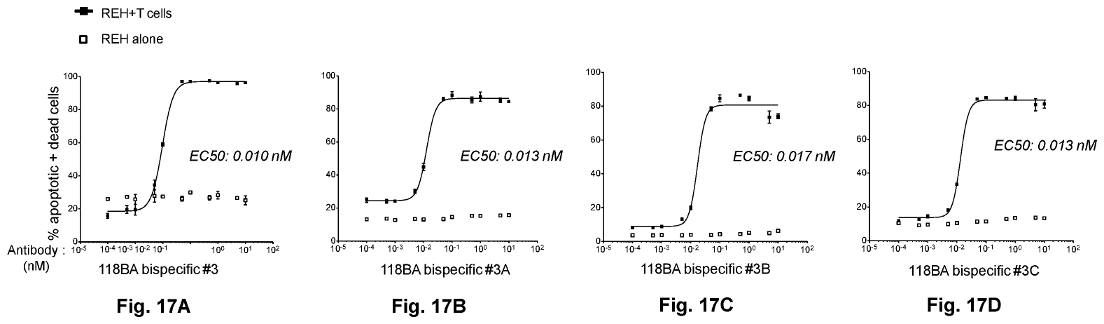

T cells from cord blood (source: New York Blood Center) were isolated using T cell isolation kit (Stemcell Technologies) and labeled with CellTrace Violet (ThermoFisher). T cells were co-cultured with FLT3 expressing REH cells (acute lymphocytic leukemia cell line) at 3:1 effector to target (E:T) ratio in the presence of 118BA #3, and its three variants 118BA #3A, 118BA #3B and 118BA #3C bispecific antibodies at various concentrations ranging from 0 nM to 100 nM. REH and T cells were also cultured separately under all conditions. Recombinant human IL2 (50 ng/ml) was added to promote T cell survival. After 3 days, cells were stained with AnnexinV, an apoptosis marker and 7-AAD, a viability dye, and analyzed by flow cytometry. Cytotoxicity was measured as % target REH cells (CellTrace Violet negative) that were dead or apoptotic for each condition. The EC50 of cytotoxicity against REH cells were determined as 0.01 nM, 0.013 nM, 0.017 nM and 0.013 nM for 118BA #3, 118BA #3A, 118BA #3B and 118BA #3C respectively as shown in FIGS. 17A-17D No toxicities were observed in REH cells when they were cultured with bispecific antibodies in the absence of T cells.

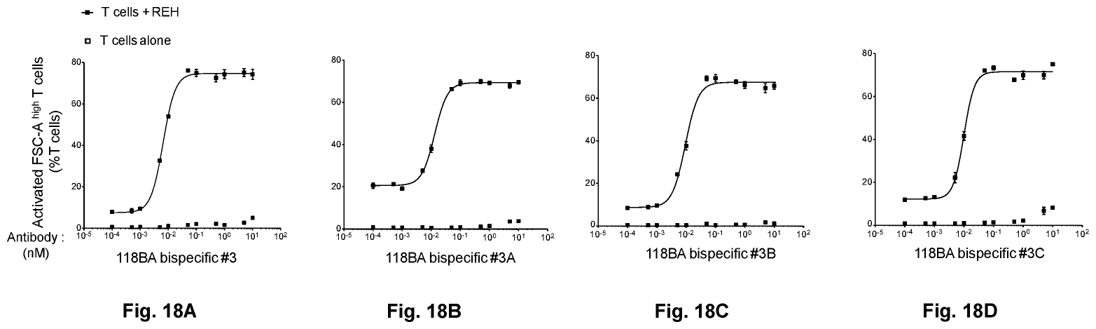

Activation of T cells by 118BA #3, 118BA #3A, 118BA #3B and 118BA #3C bispecific antibodies was determined by measuring % activated T cells identified as CellTrace Violet positive and FSC-A high for each condition as shown in FIGS. 18A-18D. Dose dependent activation of T cells was observed with bispecific antibodies 118BA #3, 118BA #3A, 118BA #3B and 118BA #3C only in the presence of target cells.

### Example 13: In Vitro Cytotoxicity and T Cell Activation of CD3×FLT3 Bispecific Antibodies 118BA #3A, and its Three Variants 118BA #3a1, 118BA #3a2 and 118BA #3a3 Towards FLT3+ REH Cells

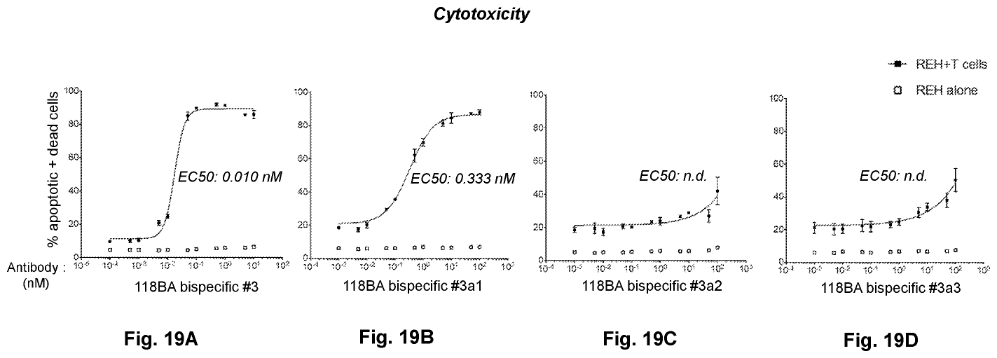

T cells from cord blood (source: New York Blood Center) were isolated using T cell isolation kit (Stemcell Technologies) and labeled with CellTrace Violet (ThermoFisher). T cells were co-cultured with FLT3 expressing REH cells (acute lymphocytic leukemia cell line) at 3:1 effector to target (E:T) ratio in the presence of 118BA #3, and its three variants 118BA #3a1 (comprising SEQ ID NOs: 17 and 19), 118BA #3a2 (comprising SEQ ID NOs: 17 and 21), and 118BA #3a3 (comprising SEQ ID NOs: 17 and 23), bispecific antibodies at various concentrations ranging from 0 nM to 100 nM. REH and T cells were also cultured separately under all conditions. Recombinant human IL2 (50 ng/ml) was added to promote T cell survival. After 3 days, cells were stained with FITC conjugated AnnexinV, an apoptosis marker and 7-AAD, a viability dye, and analyzed by flow cytometry. Cytotoxicity was measured as % target REH cells (CellTrace Violet negative) that were dead or apoptotic for each condition. The EC50 of cytotoxicity against REH cells were determined as 0.01 nM, 0.33 nM for 118BA #3 and 118BA #3a1 respectively as shown in FIGS. 19A-19D. 118BA #3a3 and #3a2 had significantly less activity compared to 118BA #3 and 118BA #3a1 and the range of concentration tested for 118BA #3a2 and #3a3 did not allow for determination of EC50 of cytotoxicity. No toxicities were observed in REH cells when they were cultured with bispecific antibodies in the absence of T cells.

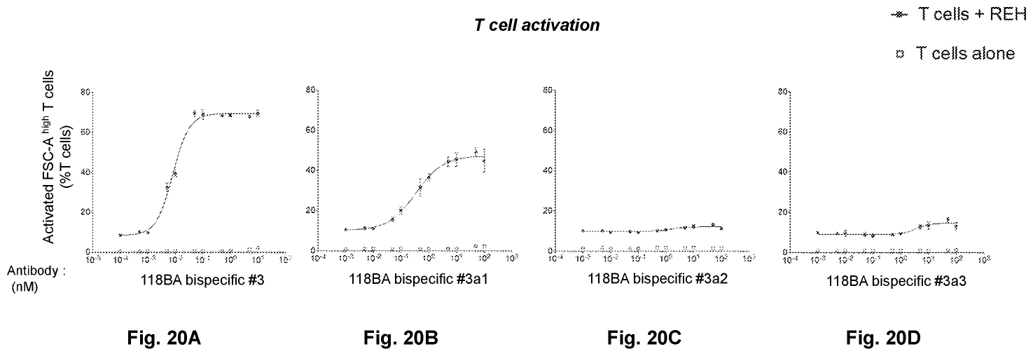

Activation of T cells by 118BA #3, 118BA #3a1, 118BA #3a2 and 118BA #3a3 bispecific antibodies was determined by measuring % activated T cells identified as CellTrace Violet positive and FSC-A high for each condition as shown in FIGS. 20A-20D. Dose dependent activation of T cells was observed with bispecific antibodies 118BA #3 and 118BA #3a1 only in the presence of target cells but was not observed for 118BA #3a2 and 118BA #3a3.

### Example 14: Evaluation of Binding Properties of 118BA #3a1 Bispecific Antibodies

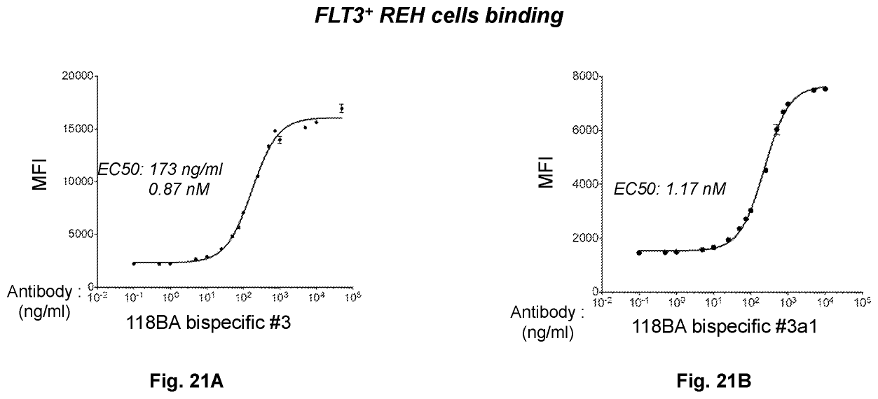

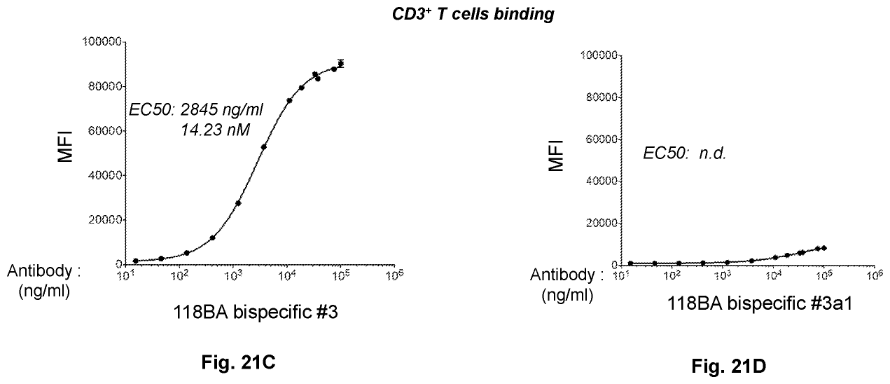

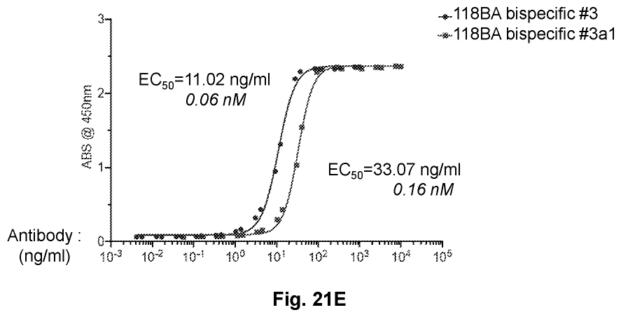

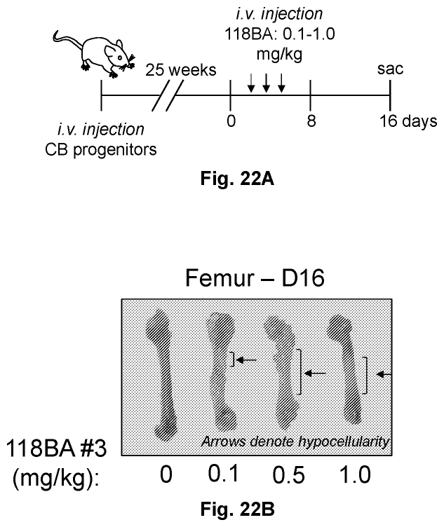

FLT3+ REH cells and CD3+ primary T cells were stained with various concentrations of 118BA #3 and 118BA #3a1 bispecific antibodies prepared in PBS+2% BCS+2 mM EDTA (flow buffer). Cells were washed five times with flow buffer and stained with anti-human IgG lambda antibody conjugated to FITC (Southern Biotech 2070-02). Cells were stained with 7-AAD (7-AAD Viability Staining Solution, Biolegend 420404) followed by flow cytometry analysis. The EC50 of binding to REH was determined to be 0.87 nM and 1.17 nM for 118BA #3 and 118BA #3a1 bispecific antibodies respectively as shown in FIGS. 21A-21B. The EC50 of binding to T cells was determined to be 14.23 nM for 118BA #3 and not determined for 118BA #3a1 with the given set of data points (FIGS. 21C-21D).

CD3e binding by 118BA #3 and 118BA #3a1 was tested by ELISA. A high-affinity protein binding 96 well plate (ThermoFisher, no. 44-2404-21) was coated with 1 μg/mL of Recombinant Human CD3 epsilon Fc Chimera Protein (R&D, no. 9850-CD-050) in PBS (Caisson Labs, no. PBL06) and incubated overnight at 4° C. The plate was washed with wash buffer (PBS (Caisson Labs, no. PBL06)+0.05% Tween20 (ThermoFisher, no. 28320)) The plate was blocked with blocking buffer (PBS (Caisson Labs, no. PBL06)+1% (w/v) BSA (Rockland, no. BSA-50)+0.05% Tween20 (ThermoFisher, no 28320)) for two hours at room temperature (RT). Plate was washed twice with wash buffer. Dilutions of 118BA #3 and 118BA #3a1 antibodies were made in PBS with concentrations ranging from 2.5*10⁻³ to 1*10⁴ ng/mL and 100 μL of the dilutions were incubated per well on the plate in duplicate for two hours at RT. Plate was washed 4× with wash buffer. Plate was incubated with 100 μL per well of 1:200 goat anti-human lambda-HRP secondary antibody (SouthernBiotech, no. 2070-05) for one hour at RT. Plate was washed 4× with wash buffer. Plate was incubated with 100 μL per well of TMB Substrate (ThermoFisher Cat #34029) for 20 minutes protected from light. Stop solution (2N H₂SO₄) was added to each well. Absorbance of wells was recorded at 450 nm on an Envision plate reader (PerkinElmer). Graph shows absorbance plotted against antibody concentration. Variable slope (four parameters) curve was fit to the data and EC50 was used to compare binding affinities. 118BA #3 has an EC50 of 11.02 ng/ml (0.06 nM) and 118BA bispecific #3a1 has an EC50 of 33.07 ng/ml (0.16 nM) (FIG. 21E).

### Example 15: 118BA #3 Humanized Mouse Bone Marrow Conditioning

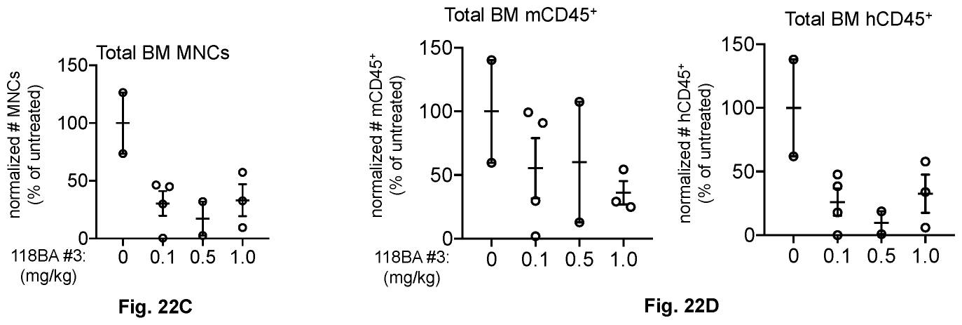

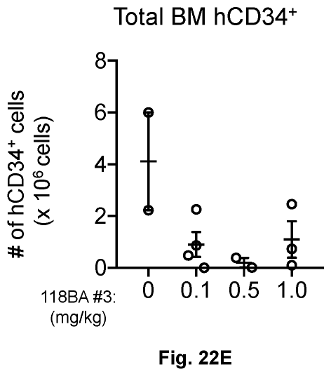

Immunocompromised mice (NOG, Taconic) were preconditioned with busulfan 24 hours prior to transplantation with human cord blood CD34⁺ hematopoietic stem and progenitor cells (HSPCs). A total of 1×10⁵ CD34⁺ HSPCs were transplanted intravenously by tail vein injection and allowed to fully engraft for 25 weeks based on detection of multilineage (human CD45+, CD3+, CD19+, CD33+) human blood cells in humanized mouse peripheral blood (>1%) by flow cytometry. At 25 weeks post-transplant, humanized mice were treated every other day for a total of 3 doses with 118BA #3 at concentrations of 0.0, 0.1, 0.5, and 1.0 mg/kg. All groups were sacrificed 16 days post-treatment for analysis of human hematopoietic cell engraftment in bone marrow (FIG. 22A).

Bone marrow from mice treated with all concentrations of 118BA #3 were observed with regions of hypocellularity, suggesting significant loss of bone marrow cells (FIG. 22B). Cell counts and flow cytometry confirm a decrease specifically in human bone marrow cells (human CD45⁺) in all treatment groups compared to control (FIGS. 22C and 22D). Further examination of human CD34⁺ HSPCs (hCD45⁺, lineage⁻, CD34⁺, CD38⁻) also revealed significant decreases in all treatment groups compared to mice receiving no 118BA #3 (FIG. 22E). As most of the FLT3 expressing cells are within the HSPC compartment, this data demonstrates that treatment with 118BA #3 leads to bone marrow conditioning of humanized mice.

### Example 16: 118BA #3 In Vivo Efficacy Against MOLM-13 AML Cell Line

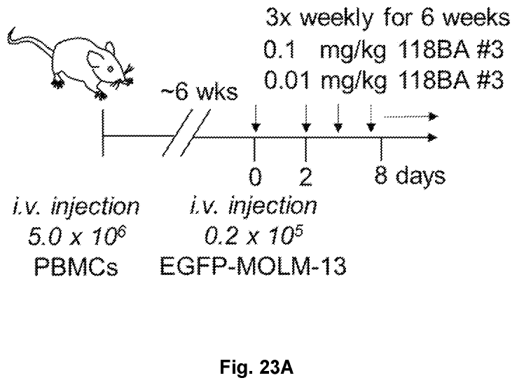

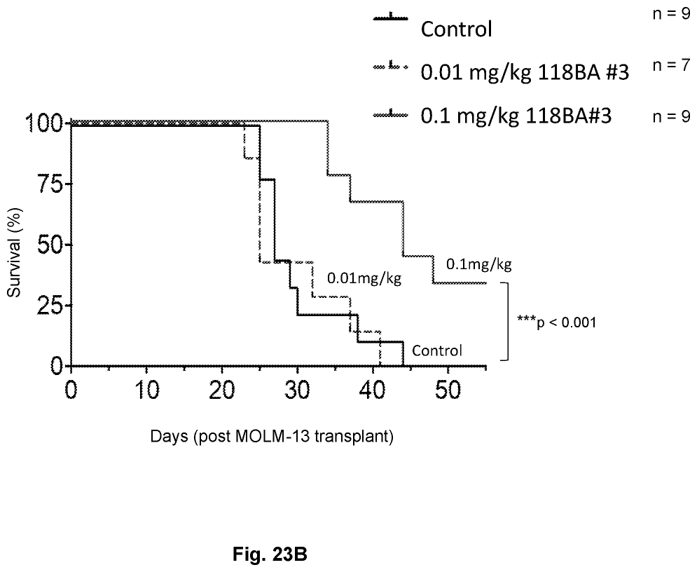

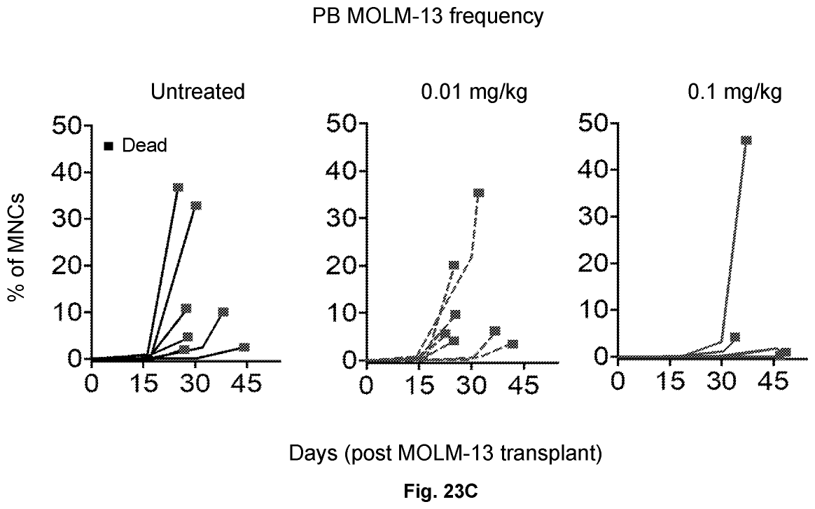

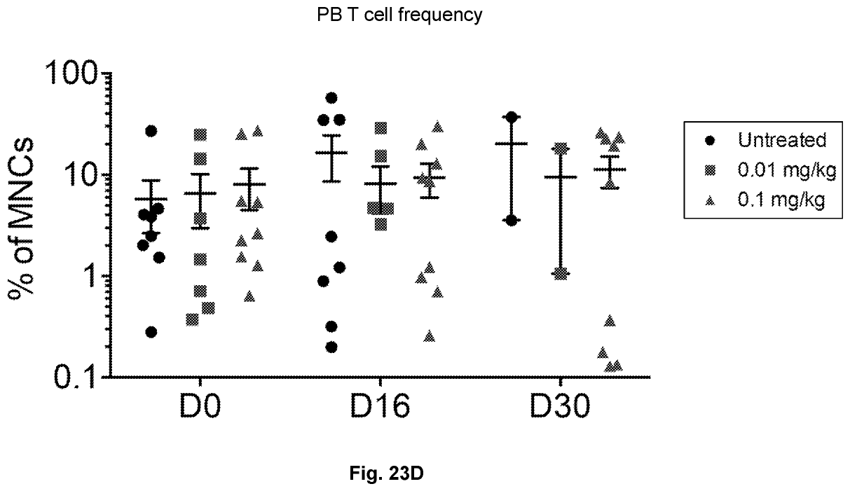

EGFP expressing MOLM-13 AML cells were xenografted into a peripheral blood mononuclear cell (PBMC) humanized mouse model that does not develop GvHD allowing for long-term survival studies and anti-leukemic T cell activity. Xenografted mice were treated 3 times per week for 6 weeks with 118BA #3 at a dose of 0.1 mg/kg or 0.01 mg/kg. Mice were sacrificed at 20% loss of body weight and/or developed hind-leg paralysis. Peripheral blood frequencies of human T cells and EGFP-MOLMs were tracked by flow cytometry every two weeks or at time of sacrifice (FIG. 23A).

Treatment with 0.1 mg/kg of 118BA #3 significantly delayed the progression of MOLM-13 cells based on peripheral blood frequencies, which was associated with an increase in median survival by 17 days compared to control (FIGS. 23B and 23C). No significant difference in survival was observed with the lower dose compared to control. Although the average human T cell frequency remained similar between all groups, a portion of mice from the 0.1 mg/kg group displayed a significant decline in T cell engraftment by day 30 associated with death by MOLM-13 progression (FIG. 23D).

### Example 17: 118BA #3 In Vivo Efficacy Against MV4-11 AML Cell Line

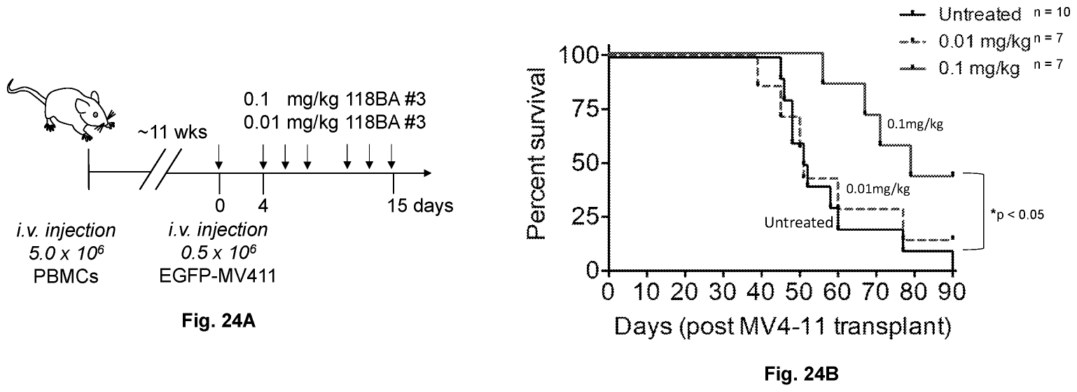

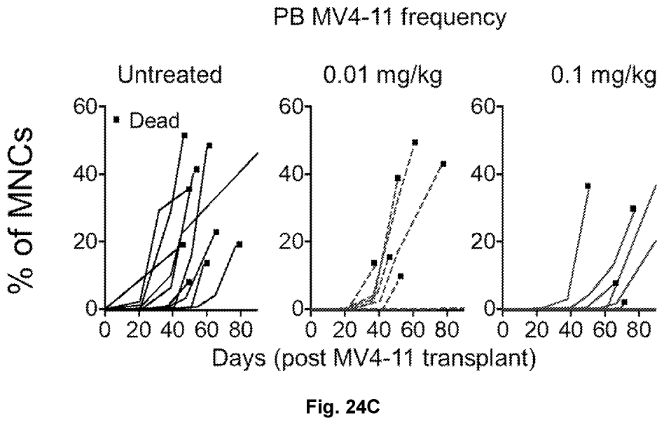

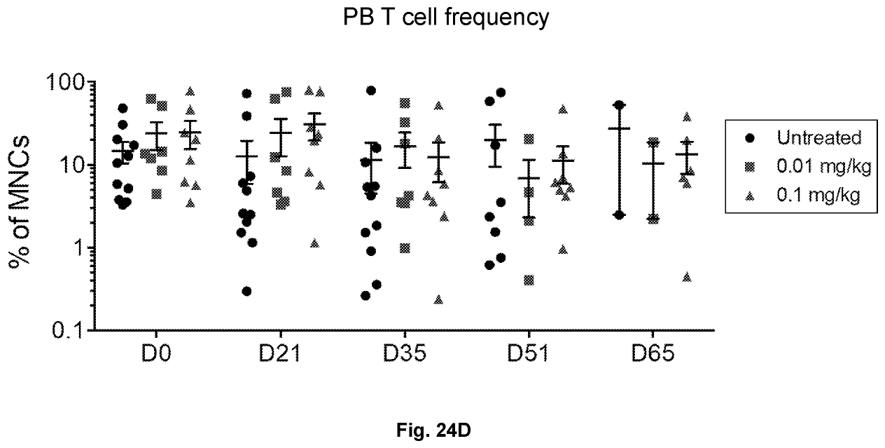

EGFP expressing MV4-11 AML cells were xenografted into a peripheral blood mononuclear cell (PBMC) humanized mouse model that does not develop GvHD allowing for long-term survival studies and anti-leukemic T cell activity. Xenografted mice were treated 3 times per week for 2 weeks with 118BA #3 at a dose of 0.1 mg/kg or 0.01 mg/kg. Mice were sacrificed at 20% loss of body weight and/or developed hind-leg paralysis Peripheral blood frequencies of human T cells and EGFP-MOLMs were tracked by flow cytometry every two weeks or at time of sacrifice (FIG. 24A).

Similar to the experiment with MOLM-13 AML cells, treatment with 0.1 mg/kg of 118BA #3 significantly delayed the progression of MV4-11 cells compared to 0.01 mg/kg and control groups, which translated to an increase in median survival by 28 days (FIGS. 24B and 24C). Average human T cell frequencies remained similar between all treatment groups but did decline over time (FIG. 24D), which was likely due to limitations in PBMC engraftment independent of 118BA #3 treatment.

### Example 18: Combination Treatment with PD1 Inhibitor

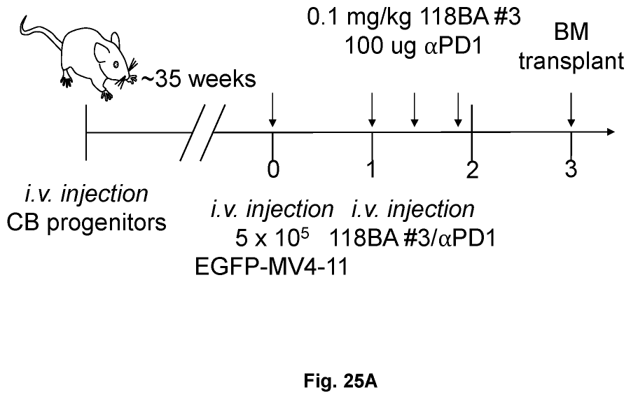

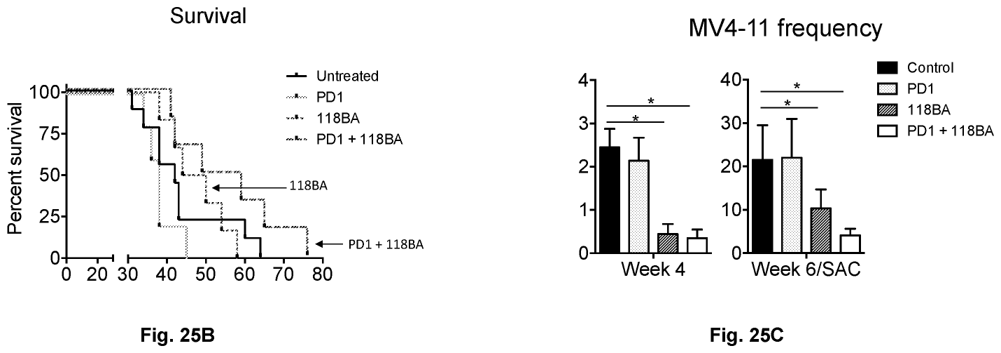

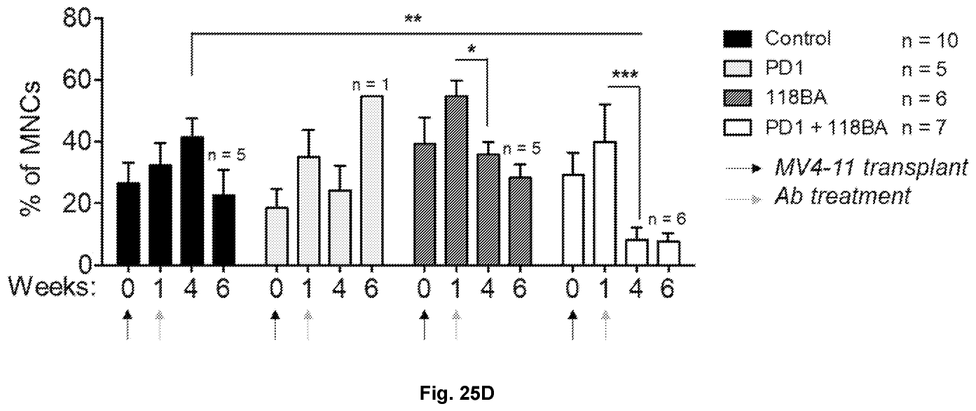

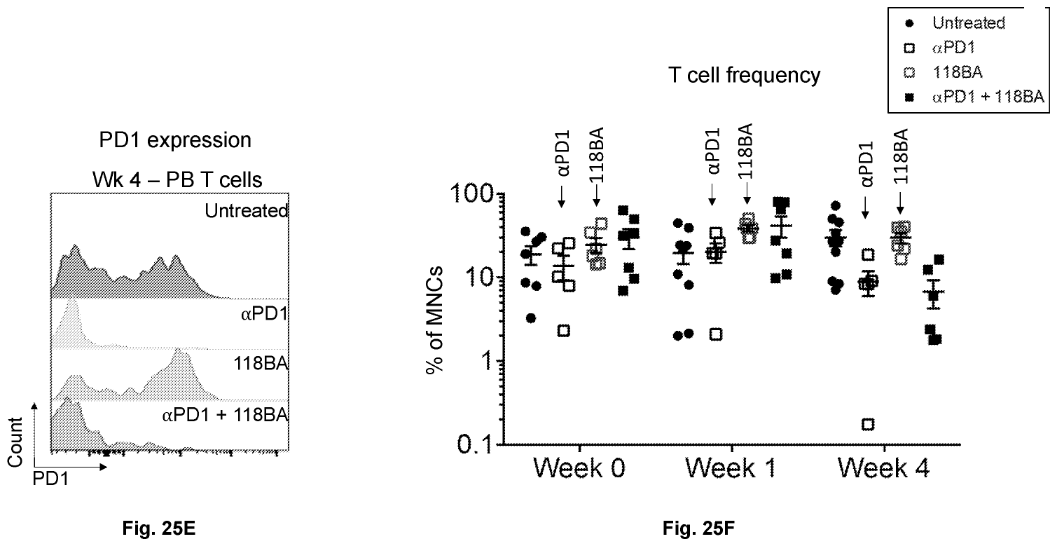

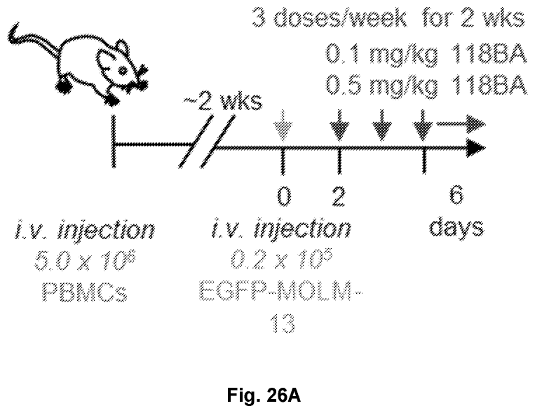

Immunocompromised mice (NOG, Taconic) were preconditioned with busulfan 24 hours prior to transplantation with human cord blood CD34⁺ hematopoietic stem and progenitor cells (HSPCs). A total of 1×10⁵ CD34⁺ HSPCs were transplanted intravenously by tail vein injection and allowed to fully engraft for 35 weeks based on detection of multilineage (human CD45+, CD3+, CD19+CD33+) human blood cells in humanized mouse peripheral blood (>1%) by flow cytometry. At 35 weeks post-transplant, mice were xenografted with EGFP-MV4-11 followed by treatment with 0.1 mg/kg 118BA #3 and/or 100 μg anti-PD1. To prevent lethality by conditioning, humanized mice were transplanted with congenic mouse BM cells 1-week post-treatment (FIG. 25A). Peripheral blood frequencies of human blood engraftment and EGFP-MV4-11 cells were tracked by flow cytometry every two weeks or at time of sacrifice.

Treatment with anti-FLT3-CD3 bispecific antibody can lead to exhaustion of T cells before full eradication of AML cells. To prevent T cell exhaustion and improve bispecific efficacy. 118BA #3 was combined with anti-PD1 to inhibit signaling through PD-L1. 118BA #3 treatment led to decreased peripheral blood frequency of EGFP-MV4-11 cells, but only modestly improved median survival (5 days) (FIGS. 25B and 25C). In contrast, co-treatment of anti-FLT3-CD3 bispecific antibody (i.e. 118BA #3) with anti-PD1 significantly reduced the burden of MV4-11 and increased median survival relative to control (12 days) (FIGS. 25B and 25C). Furthermore, administration of 118BA #3 alone or with PD1 in humanized mice also resulted in efficient elimination of the human hematopoietic compartment from mouse bone marrow (FIG. 25D). Treatment with anti-PD1 alone or with 118BA #3 was associated with a decrease in PD1 expression 3-weeks post treatment, suggesting the improvement in efficacy with combined treatment was due to preventing T cell exhaustion. However, the frequency of human T cells was significantly lower in PD1 treated mice at week 4 due to targeting of human HSPCs by 118BA #3 (FIGS. 25E-F).

### Example 19: In Vivo Comparisons of 118BA #3 Variants

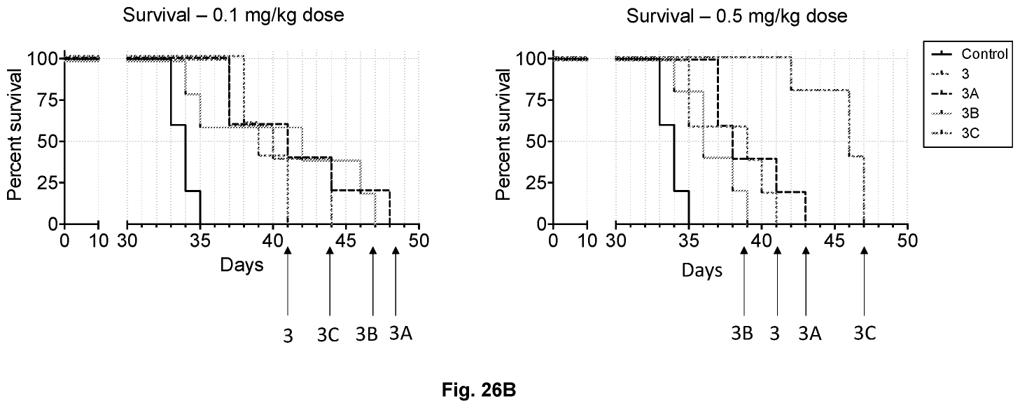

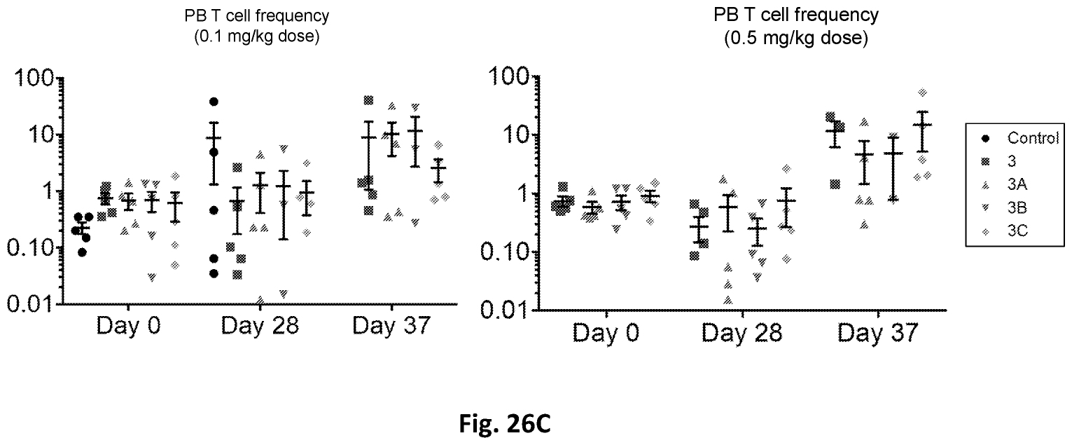

EGFP expressing MOLM-13 AML cells were xenografted into a peripheral blood mononuclear cell (PBMC) humanized mouse model that does not develop GvHD allowing for long-term survival studies and anti-leukemic T cell activity (FIG. 26A). Xenografted mice were treated 3 times per week for 2 weeks with variants of 118BA #3 (i.e. 3A, 3B, and 3C) at a dose of 0.1 mg/kg or 0.5 mg/kg. Mice were sacrificed at 20% loss of body weight and/or developed hind-leg paralysis. Peripheral blood frequencies of human T cells and EGFP-MOLMs were tracked by flow cytometry.

Variants in 118BA #3 that affect binding affinity and manufacturability were compared in vivo to determine how each variant affected cytotoxicity against AML. All variants improved survival over untreated control mice and were similar in efficacy at a dose of 0.1 mg/kg (FIG. 26B). At a dose of 0.5 mg/kg, all variants except for 3C led to a decrease in survival relative to the lower dose (FIG. 26B). Average T cell frequencies appeared similar among 118BA #3 variants at both doses (FIG. 26C). 118BA #3C at 0.5 mg/kg displayed a significant improvement in survival compared to other variants.

### Example 20: Methods of Making the Humanized Antibodies Described Herein

To generate the humanized antibodies and single-chain variable fragments described herein, the following methods were used.

### Materials and Methods

### Variable Domain Analysis and CDR Identification

For the purpose of identifying CDRs and analyzing the closest matching germline sequences the IMGT Domain Gap align tool was used: http://www.imgt.org/3Dstructure-DB/cgi/DomainGapAlign.cgi.

### Molecular Modelling

Molecular models were built for VH and VL domains based on homology to previously published antibody crystal structures using software.

### Gene Synthesis and Cloning

Variable heavy and variable light domains (for FLT3) were designed with appropriate restriction sites at the 5′ and 3′ ends to enable cloning into Absolute Antibody cloning and expression vectors. Variable domains sequences were codon optimized for expression in human cells. Following gene synthesis the variable domains were cloned into Absolute Antibody vectors of the appropriate species and type. The correct sequence was verified by Sanger sequencing with raw data analyzed using DNASTAR Lasergene software. Once confirmed plasmid DNA preps of the appropriate size were performed to generate a sufficient quantity of high quality DNA for transfection.

### Expression and Purification

Once the plasmids were generated, CHO (Chinese hamster ovary) mammalian cells were passaged to the optimum stage for transient transfection. Cells were transiently transfected with heavy and light chain expression vectors and cultured for a further 6 days. Cultures were harvested by centrifugation at 4000 rpm and filtered through a 0.22 μM filter. A first step of purification was performed by Protein A affinity chromatography with elution using citrate pH3.0 buffer followed by neutralization with 0.5M Tris, pH 9.0. Eluted protein was then buffer exchanged into PBS using a desalting column. Antibody concentration was determined by UV spectroscopy and the antibodies concentrated as necessary.

### Antibody Analytics

Antibody purity was determined by SDS-PAGE (sodium dodecyl sulphate polyacrylamide gel electrophoresis) and HPLC (high performance liquid chromatography). SEC-HPLC was performed on an Agilent 1100 series instrument using an appropriate size exclusion column (SEC). Antibody expression titre was determined by Protein A HPLC.

### Humanization of Murine Anti-FLT3

### Sequence Analysis

The VH and VL sequences for 1-18BA (the murine version of the humanized 1-18BA antibodies described herein, disclosed in US Patent Pub. No. 20190389955), which were generated using methods described in US Patent Pub. No. 20190389955 (the entirety of which is incorporated by reference herein), were run through the IGMT Gap Align tool to analyze against all known antibody germline sequences. CDR regions were assigned using the IMGT definition. The sequence is most clearly aligned to mouse, specifically the IGHV8-8 family for the VH and IGKV9-124 for the VL.

### Molecular Modelling

To enable structure guided humanization, models were built for the 1-18BA murine VH and VL sequences.

### Germline Selection

The VH and VL sequences were aligned with an Absolute Antibody database of human germline sequences.

### CDR Grafting

To humanize the antibodies, the VH and VL sequences were run through a CDR grafting algorithm to transfer the CDRs from the murine antibody 1-18BA onto the selected human germline sequences. Although CDRs are defined as being primarily responsible for binding to an antigen it is possible for amino acids outside of these regions, in what are known as framework regions, to either be involved directly in binding or to play a role in correctly orientating the CDRs. A structure guided approach was used to determine which of the framework amino acids to retain in the as the original mouse amino acid for the sake of retaining binding integrity.

### Sequence Liability Analysis

To ensure that no highly undesirable sequence liabilities had been introduced into the humanized sequences the original mouse and humanized sequences were run through an Absolute Antibody sequence liability tool.

### Antibody Production and Analytics

### Antibody Cloning

As described above, a total of 4 humanized heavy chains and 3 humanized light chains were designed. Each of these were synthesized separately and cloned as both human IgG1s and scFvs. At the point of transfection all possible combinations of the humanized sequences were made to create a total of 12 different humanized IgGs and 12 humanized scFvs.

### Antibody Expression and Purification

All antibodies were expressed at small scale and the proteins then purified by either Protein A or Nickel chromatography. All the purified products looked as expected under non-reducing and reducing SDS-PAGE.

### Aggregation Analytics

Purified IgGs were analyzed for aggregation and fragmentation by SEC-HPLC. The selected antibodies showed more than 95% monomer purity.

### Humanization of Murine Anti-CD3

### Sequence Analysis

The VH and VL sequences for SP34 (the murine version of the humanized anti-CD3 antibodies described herein, disclosed in Pessano et al., 1985, EMBO J. 4 (2): 337-344), were run through the IGMT Gap Align tool to analyze against all known antibody germline sequences. CDR regions were assigned using the IMGT definition. The sequence is most clearly aligned to mouse, specifically the IGHV10-1 family for the VH and IGLVI for the VL.

### Molecular Modelling

To enable structure guided humanization, models were built for the SP34 murine VH and VL sequences.

### Germline Selection

The VH and VL sequences were aligned with an Absolute Antibody database of human germline sequences.

### CDR Grafting

To humanize the antibodies, the VH and VL sequences were run through a CDR grafting algorithm to transfer the CDRs from the murine antibody SP34 onto the selected human germline sequences. Although CDRs are defined as being primarily responsible for binding to an antigen it is possible for amino acids outside of these regions, in what are known as framework regions, to either be involved directly in binding or to play a role in correctly orientating the CDRs. A structure guided approach was used to determine which of the framework amino acids to retain in the as the original mouse amino acid for the sake of retaining binding integrity.

### Sequence Liability Analysis

To ensure that no highly undesirable sequence liabilities had been introduced into the humanized sequences the original mouse and humanized sequences were run through an Absolute Antibody sequence liability tool.

### Antibody Production and Analytics

### Antibody Cloning

As described above, a total of 4 humanized heavy chains and 3 humanized light chains were designed. Each of these were synthesized separately and cloned into human IgG1 heavy chain and human lambda light chain expression vectors respectively. At the point of transfection all possible combinations of the humanized sequences were made to create a total of 12 different humanized antibodies.

### Antibody Expression and Purification

All antibodies were expressed at small scale and the proteins then purified by either Protein A or Nickel chromatography. All the purified products looked as expected under non-reducing and reducing SDS-PAGE.

### Aggregation Analytics

Purified antibodies were analyzed for aggregation and fragmentation by SEC-HPLC.

### Mutagenesis

The methods of antibody mutagenesis are well-known in the art.

### Example 21: Epitope Mapping

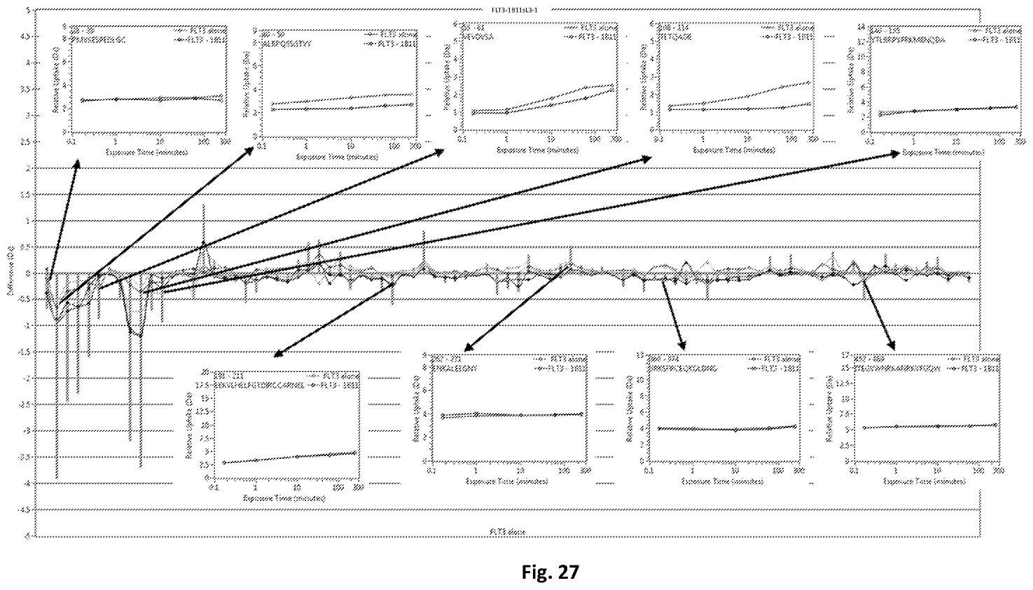

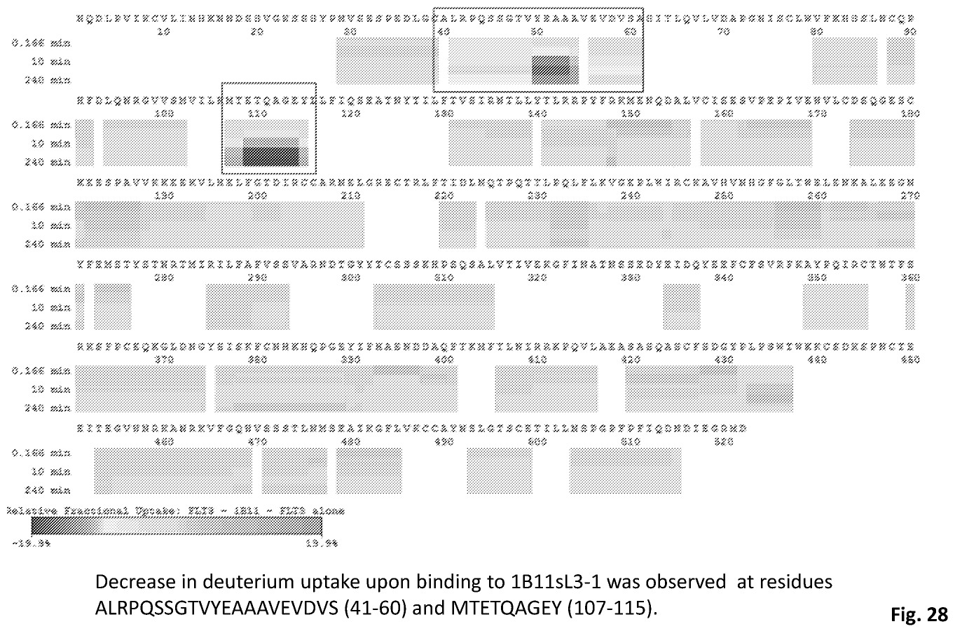

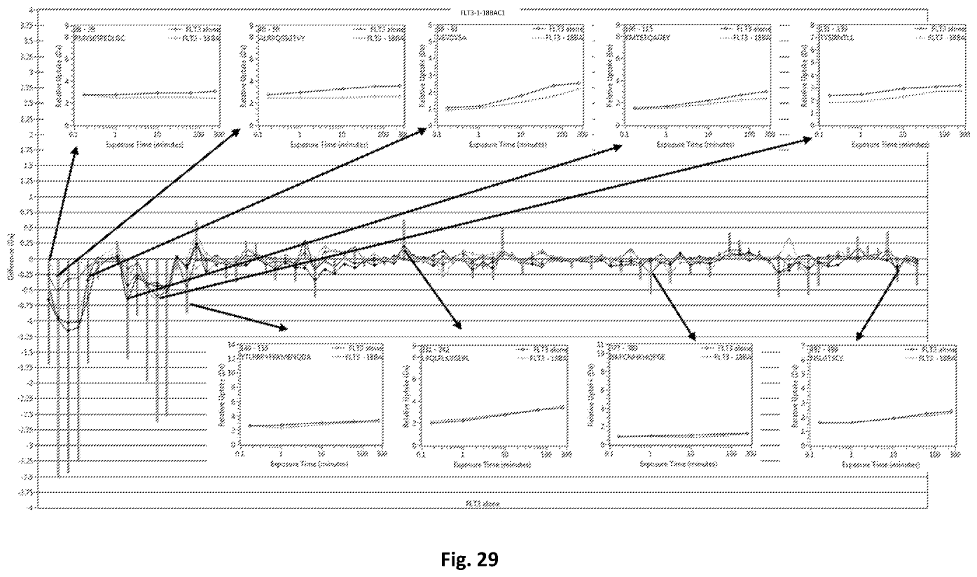

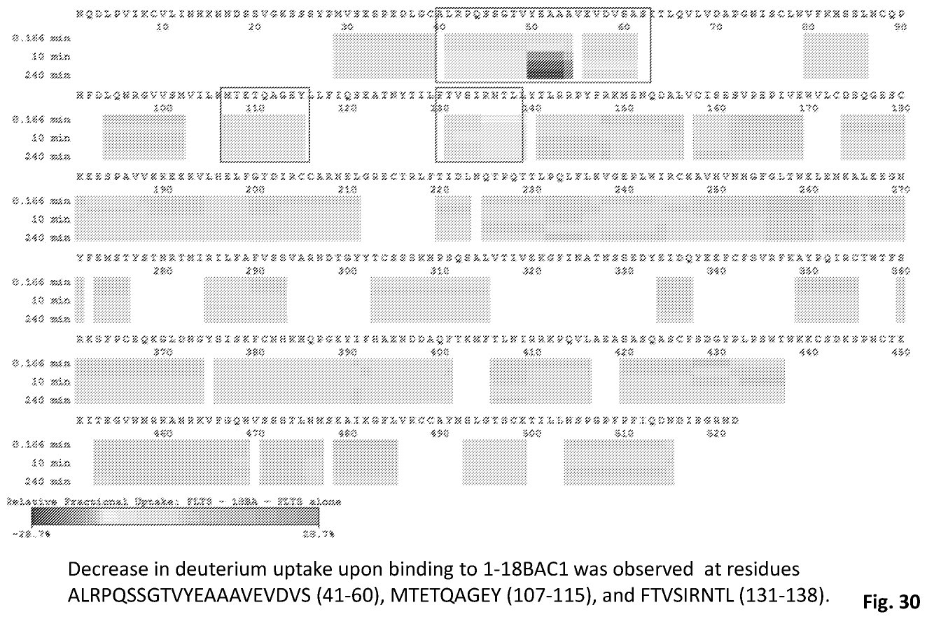

Epitope mapping for human FLT-3 was performed for 1B11sL3-1 (comprising the VH and VL of SEQ ID NOs: 97 and 98, respectively) and 1-18BAC1 (comprising the VH and VL of SEQ ID NOs: 10 and 9, respectively) both of which are humanized full-length antibodies by hydrogen exchange mass spectrometry (HDXMS). (See Balasubramaniam et al., Biochem. 2015 Mar. 3; 54 (8): 1673-1680; and Coales et al., Rapid Commun. Mass Spectrom. 2009 March; 23 (5): 639-47). Results are shown in FIGS. 27-30. For 1B11sL3-1: Decrease in deuterium uptake upon binding to 1B11sL3-1 was observed in Flt3 at residues ALRPQSSGTVYEAAAVEVDVS (41-60) (SEQ ID NO:94) and MTETQAGEY (107-115) (SEQ ID NO:95). For 1-18BAC1: Decrease in deuterium uptake upon binding to 1-18BAC1 was observed in Flt-3 at residues ALRPQSSGTVYEAAAVEVDVS (41-60) (SEQ ID NO:94), MTETQAGEY (107-115) (SEQ ID NO: 95) and FTVSIRNTL (131-138) (SEQ ID NO:96).

What is claimed is:

1. A bispecific humanized antibody or antigen binding fragment thereof that binds to human FLT3 and human CD3, wherein the antibody or fragment comprises:
(i) a first light chain variable region (VL1) comprising VL1 complementarity determining region (CDR) 1, VL1 CDR2 and VL1 CDR3, said VL1 CDR1, VL1 CDR2 and VL1 CDR3 being the CDRs of a light chain variable region (VL) that comprises the amino acid sequence of SEQ ID NO: 9; and
(ii) a first heavy chain variable region (VH1) comprising VH1 complementarity determining region (CDR) 1, VH1 CDR2 and VH1 CDR3, said VH1 CDR1, VH1 CDR2 and VH1 CDR3 being the CDRs of a heavy chain variable region (VH) that comprises the amino acid sequence of SEQ ID NO:10;
wherein the VL1 and the VH1 bind to human FLT3; and
further comprising a second VL (VL2) and a second VH (VH2) that bind to human CD3.

2. The bispecific humanized antibody or fragment of claim 1, wherein:
the VL1 CDR1 comprises the amino acid sequence QEISGY (SEQ ID NO:31), the VL1 CDR2 comprises the amino acid sequence AAS (SEQ ID NO:32), and the VL1 CDR3 comprises the amino acid sequence LQYASYPLT (SEQ ID NO:37); and
the VH1 CDR1 comprises the amino acid sequence GFSLSRSTMG (SEQ ID NO:38), the VH1 CDR2 comprises the amino acid sequence IKWNDSK (SEQ ID NO:39), and the VH1 CDR3 comprises the amino acid sequence ARIVYYSTYVGYFDV (SEQ ID NO:36).

3. The bispecific humanized antibody or fragment of claim 1, wherein:
the VL1 CDR1 comprises the amino acid sequence RASQEISGYLS (SEQ ID NO:71), the VL1 CDR2 comprises the amino acid sequence AASTLHS (SEQ ID NO:72), and the VL1 CDR3 comprises the amino acid sequence LQYASYPLT (SEQ ID NO:37); and
the VH1 CDR1 comprises the amino acid sequence GFSLSRSTMGVG (SEQ ID NO:73), the VH1 CDR2 comprises the amino acid sequence HIKWNDSKYYNPALKS (SEQ ID NO:74), and the VH1 CDR3 comprises the amino acid sequence IVYYSTYVGYFDV (SEQ ID NO:75).

4. The bispecific humanized antibody or fragment of any one of claims 1-3, wherein:
(iii) the VL2 comprises VL2 CDR1, VL2 CDR2 and VL2 CDR3, said VL2 CDR1, VL2 CDR2 and VL2 CDR3 being the CDRs of a VL that comprises an amino acid sequence selected from the group consisting of: SEQ ID NO:4, SEQ ID NO:6, SEQ ID NO:14, SEQ ID NO: 16, SEQ ID NO: 24, and SEQ ID NO.29, and
(iv) the VH2 comprises VH2 CDR1, VH2 CDR2 and VH2 CDR3, said VH2 CDR1, VH2 CDR2 and VH2 CDR3 being the CDRs of a VH that comprises an amino acid sequence selected from the group consisting of: SEQ ID NO:5, SEQ ID NO:7, SEQ ID NO: 12, SEQ ID NO: 18, SEQ ID NO: 20 and SEQ ID NO:22;
wherein the VL2 and the VH2 bind to human CD3.

5. The bispecific humanized antibody or fragment of claim 4, wherein:
the VL2 CDR1 comprises the amino acid sequence of TGAVTTSNY (SEQ ID NO:40), the VL2 CDR2 comprises the amino acid sequence of GTN (SEQ ID NO:41), and the VL2 CDR3 comprises an amino acid sequence selected from the group consisting of: ALWYSNLWV (SEQ ID NO: 42), ALWFSNHWV (SEQ ID NO:46), and ALWYSNHWV (SEQ ID NO:47); and
the VH2 CDR1 comprises the amino acid sequence of GFTFNTYA (SEQ ID NO:43), the VH2 CDR2 comprises the amino acid sequence of IRSKYNNYAT (SEQ ID NO:44), and the VH2 CDR3 comprises an amino acid sequence selected from the group consisting of: HGNFGNSYVSWFAY (SEQ ID NO:83), HGNFGTSYVSWFAY (SEQ ID NO:84), HGHFGTSYVSWFAY (SEQ ID NO:85), HGMFGTSYVSWFAY (SEQ ID NO.86), and HGQFGTSYVSWFAY (SEQ ID NO:87).

6. The bispecific humanized antibody or fragment of claim 4, wherein:
the VL2 CDR1 comprises the amino acid sequence of GSSTGAVTTSNYAN (SEQ ID NO: 76), the VL2 CDR2 comprises an amino acid sequence selected from the group consisting of: GTNKRSS (SEQ ID NO:77), GTNKRVS (SEQ ID NO:78), and GINKRSS (SEQ ID NO:79) and GTNKRAS (SEQ ID NO:80); and the VL2 CDR3; and the VL2 CDR3 comprises an amino acid sequence selected from the group consisting of: ALWYSNLWV (SEQ ID NO:42), ALWFSNHWV (SEQ ID NO:46), and ALWYSNHWV (SEQ ID NO:47); and
the VH2 CDR1 comprises the amino acid sequence of GFTFNTYAMN (SEQ ID NO:81), the VH2 CDR2 comprises the amino acid sequence of RIRSKYNNYATYYADSVKG (SEQ ID NO: 82), and the VH2 CDR3 comprises an amino acid sequence selected from the group consisting of: HGNFGNSYVSWFAY (SEQ ID NO:83), HGNFGTSYVSWFAY (SEQ ID NO:84), HGHFGTSYVSWFAY (SEQ ID NO:85), HGMFGTSYVSWFAY (SEQ ID NO:86), and HGQFGTSYVSWFAY (SEQ ID NO:87).

7. The bispecific humanized antibody or fragment of claim 4, wherein
the VL2 CDR1, VL2 CDR2 and VL2 CDR3 are the CDRs of a VL that comprises the amino acid sequence of SEQ ID NO:4, and/or the VL2 CDR1 comprises the amino acid sequence TGAVTTSNY (SEQ ID NO:40), the VL2 CDR2 comprises the amino acid sequence GTN (SEQ ID NO: 41), and the VL2 CDR3 comprises the amino acid sequence ALWYSNLWV (SEQ ID NO: 42); and
the VH2 CDR1, VH2 CDR2 and VH2 CDR3 are the CDRs of a VH that comprises the amino acid sequence of SEQ ID NO:5, and/or the VH2 CDR1 comprises the amino acid sequence GFTFNTYA (SEQ ID NO:43), the VH2 CDR2 comprises the amino acid sequence IRSKYNNYAT (SEQ ID NO:44), and the VH2 CDR3 comprises the amino acid sequence HGNFGNSYVSWFAY (SEQ ID NO:83).

8. The bispecific humanized antibody or fragment of claim 4, wherein
the VL2 CDR1, VL2 CDR2 and VL2 CDR3 are the CDRs of a VL that comprises the amino acid sequence of SEQ ID NO:4, and/or the VL2 CDR1 comprises the amino acid sequence TGAVTTSNY (SEQ ID NO:40), the VL2 CDR2 comprises the amino acid sequence GTN (SEQ ID NO: 41), and the VL CDR3 comprises the amino acid sequence ALWYSNLWV (SEQ ID NO: 42); and
the VH2 CDR1, VH2 CDR2 and VH2 CDR3 are the CDRs of a VH that comprises the amino acid sequence of SEQ ID NO: 12, and/or the VH2 CDR1 comprises the amino acid sequence GFTFNTYA (SEQ ID NO:43), the VH2 CDR2 comprises the amino acid sequence IRSKYNNYAT (SEQ ID NO:44), and the VH2 CDR3 comprises the amino acid sequence VRHGNFGTSYVSWFAY (SEQ ID NO:48).

9. The bispecific humanized antibody or fragment of claim 4, wherein
the VL2 CDR1, VL2 CDR2 and VL2 CDR3 are the CDRs of a VL that comprises an amino acid sequence of SEQ ID NO:14 or SEQ ID NO:16, and/or the VL2 CDR1 comprises the amino acid sequence TGAVTTSNY (SEQ ID NO:40), the VL2 CDR2 comprises the amino acid sequence GTN (SEQ ID NO:41), and the VL CDR3 comprises the amino acid sequence ALWFSNHWV (SEQ ID NO:46); and
the VH2 CDR1, VH2 CDR2 and VH2 CDR3 are the CDRs of a VH that comprises the amino acid sequence of SEQ ID NO: 12, and/or the VH2 CDR1 comprises the amino acid sequence GFTFNTYA (SEQ ID NO:43), the VH2 CDR2 comprises the amino acid sequence IRSKYNNYAT (SEQ ID NO:44), and the VH2 CDR3 comprises the amino acid sequence VRHGNFGTSYVSWFAY (SEQ ID NO:45).

10. The bispecific humanized antibody or fragment of claim 4, wherein
the VL2 CDR1, VL2 CDR2 and VL2 CDR3 are the CDRs of a VL that comprises the amino acid sequence of SEQ ID NO: 14, and/or the VL2 CDR1 comprises the amino acid sequence TGAVTTSNY (SEQ ID NO:40), the VL2 CDR2 comprises the amino acid sequence GTN (SEQ ID NO: 41), and the VL CDR3 comprises the amino acid sequence ALWFSNHWV (SEQ ID NO: 46); and
the VH2 CDR1, VH2 CDR2 and VH2 CDR3 are the CDRs of a VH that comprises the amino acid sequence of SEQ ID NO: 18, and/or the VH2 CDR1 comprises the amino acid sequence GFTENTYA (SEQ ID NO:43), the VH2 CDR2 comprises the amino acid sequence IRSKYNNYAT (SEQ ID NO:44), and the VH2 CDR3 comprises the amino acid sequence VRHGHFGTSYVSWFAY (SEQ ID NO:49).

11. The bispecific humanized antibody or fragment of claim 4, wherein
the VL2 CDR1, VL2 CDR2 and VL2 CDR3 are the CDRs of a VL that comprises the amino acid sequence of SEQ ID NO: 14, and/or the VL2 CDR1 comprises the amino acid sequence TGAVTTSNY (SEQ ID NO:40), the VL2 CDR2 comprises the amino acid sequence GTN (SEQ ID NO: 41), and the VL CDR3 comprises the amino acid sequence ALWFSNHWV (SEQ ID NO: 46); and
the VH2 CDR1, VH2 CDR2 and VH2 CDR3 are the CDRs of a VH that comprises the amino acid sequence of SEQ ID NO:20, and/or the VH2 CDR1 comprises the amino acid sequence GFTFNTYA (SEQ ID NO:43), the VH2 CDR2 comprises the amino acid sequence IRSKYNNYAT (SEQ ID NO:44), and the VH2 CDR3 comprises the amino acid sequence VRHGMFGTSYVSWFAY (SEQ ID NO:50).

12. The bispecific humanized antibody or fragment of claim 4, wherein
the VL2 CDR1, VL2 CDR2 and VL2 CDR3 are the CDRs of a VL that comprises the amino acid sequence of SEQ ID NO:14, and/or the VL2 CDR1 comprises the amino acid sequence TGAVTTSNY (SEQ ID NO:40), the VL2 CDR2 comprises the amino acid sequence GTN (SEQ ID NO: 41), and the VL CDR3 comprises the amino acid sequence ALWFSNHWV (SEQ ID NO: 46); and
the VH2 CDR1, VH2 CDR2 and VH2 CDR3 are the CDRs of a VH that comprises the amino acid sequence of SEQ ID NO:22, and/or the VH2 CDR1 comprises the amino acid sequence GFTENTYA (SEQ ID NO:43), the VH2 CDR2 comprises the amino acid sequence IRSKYNNYAT (SEQ ID NO:44), and the VH2 CDR3 comprises the amino acid sequence VRHGQFGTSYVSWFAY (SEQ ID NO:51).

13. The bispecific humanized antibody or fragment of claim 4, wherein
the VL2 CDR1, VL2 CDR2 and VL2 CDR3 are the CDRs of a VL that comprises the amino acid sequence of SEQ ID NO:24 or SEQ ID NO:29, and/or the VL2 CDR1 comprises the amino acid sequence TGAVTTSNY (SEQ ID NO:40), the VL2 CDR2 comprises the amino acid sequence GTN (SEQ ID NO:41), and the VL CDR3 comprises the amino acid sequence ALWYSNHWV (SEQ ID NO:47); and
the VH2 CDR1, VH2 CDR2 and VH2 CDR3 are the CDRs of a VH that comprises the amino acid sequence of SEQ ID NO:5, and/or the VH2 CDR1 comprises the amino acid sequence GFTFNTYA (SEQ ID NO:43), the VH2 CDR2 comprises the amino acid sequence IRSKYNNYAT (SEQ ID NO:44), and the VH2 CDR3 comprises the amino acid sequence VRHGNFGNSYVSWFAY (SEQ ID NO:45).

14. A bispecific humanized antibody or antigen binding fragment thereof that binds to human FLT3 and human CD3, wherein the antibody or fragment comprises:
a VL2 comprising VL2 CDR1, VL2 CDR2 and VL2 CDR3, said VL2 CDR1, VL2 CDR2 and VL2 CDR3 being the CDRs of a VL that comprises an amino acid sequence selected from the group consisting of: SEQ ID NO: 14, SEQ ID NO: 16, SEQ ID NO:24, and SEQ ID NO:29; and
a VH2 comprising VH2 CDR1, VH2 CDR2 and VH2 CDR3, said VH2 CDR1, VH2 CDR2 and VH2 CDR3 being the CDRs of a VH that comprises an amino acid sequence selected from the group consisting of: SEQ ID NO: 12, SEQ ID NO:18, SEQ ID NO:20, and SEQ ID NO:22;
wherein the VL2 and the VH2 bind to human CD3; and
further comprising a VL1 and a VH1 that bind to human FLT3.

15. The bispecific humanized antibody or fragment of claim 14, wherein:
the VL2 CDR1 comprises the amino acid sequence TGAVTTSNY (SEQ ID NO:40), the VL2 CDR2 comprises the amino acid sequence GTN (SEQ ID NO:41), and the VL2 CDR3 comprises the amino acid sequence ALWFSNHWV (SEQ ID NO:46) or ALWYSNHWV (SEQ ID NO: 47); and
the VH2 CDR1 comprises the amino acid sequence GFTFNTYA (SEQ ID NO:43), the VH2 CDR2 comprises the amino acid sequence IRSKYNNYAT (SEQ ID NO:44), and the VH2 CDR3 comprises an amino acid sequence selected from the group consisting of: VRHGNFGTSYVSFAY (SEQ ID NO:48), VRHGHFGTSYVSFAY (SEQ ID NO:49), VRHGMFGTSYVSFAY (SEQ ID NO:50), and VRHGQFGTSYVSFAY (SEQ ID NO:51).

16. The bispecific humanized antibody or fragment of any one of claims 1-15, wherein the VL1 comprises the amino acid sequence of SEQ ID NO:9.

17. The bispecific humanized antibody or fragment of any one of claims 1-16, wherein the VH1 comprises the amino acid sequence of SEQ ID NO:10.

18. The bispecific humanized antibody or fragment of any one of claims 1-17, which comprises a single chain variable fragment (scFv), wherein the scFv comprises the VL1 and the VH1, and wherein the scFv comprises the amino acid sequence of SEQ ID NO:52.

19. The bispecific humanized antibody or fragment of any one of claims 1-18, wherein the antibody or fragment comprise a heavy chain (HC) and a light chain (LC), wherein the HC comprises the VL1, the VH1, and the VH2.

20. The bispecific humanized antibody or fragment of claim 19, wherein the VL1 is joined to the VH1 by a first linker, and wherein the VH1 is joined to the VH2 by a second linker; optionally, wherein the C-terminus of the VL1 is joined to the N-terminus of the VH1 by a first linker, and wherein the C-terminus of the VH1 is joined to the N-terminus of the VH2 by a second linker.

21. The bispecific humanized antibody or fragment of claim 20, wherein the first linker and the second linker have the formula (Gly₃₋₄-Ser)₁₋₄.

22. The bispecific humanized antibody or fragment of claim 21, wherein the first linker has the formula (Gly₄-Ser)₄.

23. The bispecific humanized antibody or fragment claim 21 or 22, wherein the second linker has the formula (Gly₄-Ser)₃.

24. The bispecific humanized antibody or fragment of claim 19, wherein the HC comprises an amino acid sequence selected from the group consisting of: SEQ ID NO:53, SEQ ID NO:54, SEQ ID NO:55, SEQ ID NO:56 and SEQ ID NO:57.

25. The bispecific humanized antibody or fragment of claim 24, wherein the HC comprises the amino acid sequence of SEQ ID NO:53.

26. The bispecific humanized antibody or fragment of claim 24, wherein the HC comprises the amino acid sequence of SEQ ID NO:54.

27. The bispecific humanized antibody or fragment of claim 24, wherein the HC comprises the amino acid sequence of SEQ ID NO:55.

28. The bispecific humanized antibody or fragment of claim 24, wherein the HC comprises the amino acid sequence of SEQ ID NO:56.

29. The bispecific humanized antibody or fragment of claim 24, wherein the HC comprises the amino acid sequence of SEQ ID NO:57.

30. The bispecific humanized antibody or fragment of any one of claims 19-29, wherein the LC comprises the VH2.

31. The bispecific humanized antibody or fragment of claim 30, wherein the LC comprises an amino acid sequence selected from the group consisting of: SEQ ID NO:4, SEQ ID NO:14, SEQ ID NO: 16, SEQ ID NO:24 and SEQ ID NO:29.

32. The bispecific humanized antibody or fragment of claim 31, wherein the LC comprises the amino acid sequence of SEQ ID NO:4.

33. The bispecific humanized antibody or fragment of claim 31, wherein the LC comprises the amino acid sequence of SEQ ID NO:14.

34. The bispecific humanized antibody or fragment of claim 31, wherein the LC comprises the amino acid sequence of SEQ ID NO:16.

35. The bispecific humanized antibody or fragment of claim 31, wherein the LC comprises the amino acid sequence of SEQ ID NO:24.

36. The bispecific humanized antibody or fragment of claim 31, wherein the LC comprises the amino acid sequence of SEQ ID NO:29.

37. A bispecific humanized antibody or antigen binding fragment thereof that binds to human FLT3 and human CD3, wherein the antibody or fragment comprises:
(i) a first light chain variable region (VL1), wherein the VL1 comprises the amino acid sequence of SEQ ID NO:1; and/or
(ii) a first heavy chain variable region (VH1), wherein the VH1 comprises the amino acid sequence of SEQ ID NO:2;
wherein the VL1 and the VH1 bind to human FLT3; and
further comprising a second light chain variable region (VL2) and a second heavy chain variable region (VH2) that bind to human CD3.

38. The bispecific humanized antibody or fragment of claim 37, wherein the VL1 has the amino acid sequence of SEQ ID NO: 1, and the VH1 has the amino acid sequence of SEQ ID NO: 2.

39. The bispecific humanized antibody or fragment claim 37, which comprises a single chain variable fragment (scFv), wherein the scFv comprises the VL1 and the VH1, and wherein the scFv comprises the amino acid sequence of SEQ ID NO:3.

40. The bispecific humanized antibody or fragment of any one of claims 37-39, wherein:
(iii) the VL2 comprises the amino acid sequence of SEQ ID NO:4 or SEQ ID NO:6; and
(iv) the VH2 comprises the amino acid sequence of SEQ ID NO:5 or SEQ ID NO:7.

41. The bispecific humanized antibody or fragment of claim 40, wherein the VL2 has the amino acid sequence of SEQ ID NO:4, and the VH2 has the amino acid sequence of SEQ ID NO:5.

42. The bispecific humanized antibody or fragment of claim 40, wherein the VL2 has the amino acid sequence of SEQ ID NO:6, and the VH2 has the amino acid sequence of SEQ ID NO:7.

43. A bispecific humanized antibody or antigen binding fragment thereof that binds to human FLT3 and human CD3, wherein the antibody or fragment comprises:
(i) a VL1 that comprises the amino acid sequence of SEQ ID NO:4 or SEQ ID NO:6; and
(ii) a VH1 that comprises the amino acid sequence of SEQ ID NO:5 or SEQ ID NO:7;
wherein the VL1 and the VH1 bind to human CD3; and
further comprising a VL2 and a VH2 that bind to human FLT3.

44. The bispecific humanized antibody or fragment of claim 43, wherein the VL1 has the amino acid sequence of SEQ ID NO:4 and the VH1 has the amino acid sequence of SEQ ID NO:5.

45. The bispecific humanized antibody or fragment of claim 43, wherein the VL1 has the amino acid sequence of SEQ ID NO:6 and the VH1 has the amino acid sequence of SEQ ID NO:7.

46. The bispecific humanized antibody or fragment of any one of claims 37-45, wherein the antibody or fragment comprise a heavy chain (HC) and a light chain (LC).

47. The bispecific humanized antibody or fragment of any one of claims 19-36 and 46, wherein the LC comprises a constant domain.

48. The bispecific humanized antibody or fragment of claim 47, where the constant domain comprises the amino acid sequence of SEQ ID NO:58.

49. The bispecific humanized antibody or fragment of any one of claims 19-36 and 46-48, wherein the HC comprises an Fc region.

50. The bispecific humanized antibody or fragment of claim 49, wherein the Fc region is an IgG.

51. The bispecific humanized antibody or fragment of claim 50, wherein the IgG is a human IgG1, a human IgG2, a human IgG3, or a human IgG4.

52. The bispecific humanized antibody or fragment of claim 49, wherein the Fc region comprises the amino acid sequence of SEQ ID NO:59.

53. The bispecific humanized antibody or fragment of claim 49, wherein the Fc region comprises the amino acid sequence of SEQ ID NO:27.

54. The bispecific humanized antibody of claim 1 or 4, wherein the antibody comprises an LC comprising the amino acid sequence of SEQ ID NO:8, and an HC comprising the amino acid sequence of SEQ ID NO:11.

55. The bispecific humanized antibody of claim 1 or 4, wherein the antibody comprises an LC comprising the amino acid sequence of SEQ ID NO:8, and an HC comprising the amino acid sequence of SEQ ID NO:13.

56. The bispecific humanized antibody of claim 1 or 4, wherein the antibody comprises an LC comprising the amino acid sequence of SEQ ID NO:15, and an HC comprising the amino acid sequence of SEQ ID NO:13.

57. The bispecific humanized antibody of claim 1 or 4, wherein the antibody comprises an LC comprising the amino acid sequence of SEQ ID NO:17, and an HC comprising the amino acid sequence of SEQ ID NO:13.

58. The bispecific humanized antibody of claim 1 or 4, wherein the antibody comprises an LC comprising the amino acid sequence of SEQ ID NO:17, and an HC comprising the amino acid sequence of SEQ ID NO:19.

59. The bispecific humanized antibody of claim 1 or 4, wherein the antibody comprises an LC comprising the amino acid sequence of SEQ ID NO:17, and an HC comprising the amino acid sequence of SEQ ID NO:21.

60. The bispecific humanized antibody of claim 1 or 4, wherein the antibody comprises an LC comprising the amino acid sequence of SEQ ID NO:17, and an HC comprising the amino acid sequence of SEQ ID NO:23.

61. The bispecific humanized antibody of claim 1 or 4, wherein the antibody comprises an LC comprising the amino acid sequence of SEQ ID NO:25, and an HC comprising the amino acid sequence of SEQ ID NO:28.

62. The bispecific humanized antibody of claim 1 or 4, wherein the antibody comprises an LC comprising the amino acid sequence of SEQ ID NO:30, and an HC comprising the amino acid sequence of SEQ ID NO:28.

63. The bispecific humanized antibody or fragment of any one of claims 1-62, which is a monoclonal antibody.

64. The bispecific humanized antibody or fragment of any one of claims 1-63, wherein the antibody or fragment is purified.

65. The bispecific humanized antibody or fragment of any one of claims 1-64, wherein the antibody or fragment has half-life of 1 day to 14 days in a human.

66. The bispecific humanized antibody or fragment of claim 65, wherein the antibody or fragment has half-life of 4 days to 7 days in a human.

67. A pharmaceutical composition comprising a therapeutically effective amount of the antibody or fragment of any one of claims 1-66 and a pharmaceutically acceptable excipient.

68. The pharmaceutical composition of claim 67, which further comprises an anti-tumor agent.

69. A method of treating a hematologic cancer in a subject in need thereof, wherein the method comprises administering to the subject a therapeutically effective amount of: (i) an antibody or fragment of any one of claims 1-66, or (ii) the pharmaceutical composition of claim 67 or 68.

70. The method of claim 69, where the hematologic cancer is acute myeloid leukemia (AML), acute lymphoblastic leukemia (ALL), chronic myeloid leukemia (CML), chronic lymphocytic leukemia (CLL), peripheral T cell lymphoma, follicular lymphoma, diffuse large B cell lymphoma, Hodgkin lymphoma, non-Hodgkin lymphoma, neuroblastoma, a non-malignant inherited or acquired marrow disorder, multiple myeloma, a dendritic cell neoplasm, or blastic plasmacytoid dendritic cell neoplasm (BPDCN).

71. The method of claim 69, wherein the hematologic cancer is AML.

72. The method of claim 69, wherein the hematologic cancer is a dendritic cell neoplasm.

73. The method of claim 69, wherein the hematologic cancer is blastic plasmacytoid dendritic cell neoplasm (BPDCN).

74. The method of claim 69, wherein the hematologic cancer is a non-malignant inherited or acquired marrow disorder, and wherein the non-malignant inherited or acquired marrow disorder is selected from sickle anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, or congenital thrombocytopenia.

75. A method for preparing or conditioning a subject in need thereof for hematopoietic cell transplantation, wherein the method comprises administering to the subject a therapeutically effective amount of: (i) an antibody or fragment of any one of claims 1-66, or (ii) the pharmaceutical composition of claim 67 or 68; and wherein the administering occurs prior to the hematopoietic cell transplantation.

76. The method of claim 75, wherein the therapeutically effective amount reduces the cell population expressing one or more of CD34, FLT3, CD33, CD11b, CD16, CD15, and CD66b by at least 90%.

77. The method of claim 75, wherein the therapeutically effective amount reduces the cell population expressing FLT3 and CD34 by at least 90%.

78. The method of any one of claims 75 and 77, wherein the subject in need thereof has a hematologic cancer.

79. The method of claim 78, wherein the hematologic cancer is acute myeloid leukemia (AML), acute lymphoblastic leukemia (ALL), chronic myeloid leukemia (CML), chronic lymphocytic leukemia (CLL), peripheral T cell lymphoma, follicular lymphoma, diffuse large B cell lymphoma, Hodgkin lymphoma, non-Hodgkin lymphoma, neuroblastoma, a non-malignant inherited or acquired marrow disorder, multiple myeloma, a dendritic cell neoplasm, or blastic plasmacytoid dendritic cell neoplasm (BPDCN).

80. The method of claim 78, wherein the hematologic cancer is AML.

81. The method of claim 78, wherein the hematologic cancer is a dendritic cell neoplasm.

82. The method of claim 78, wherein the hematologic cancer is blastic plasmacytoid dendritic cell neoplasm (BPDCN).

83. The method of claim 78, wherein the hematologic cancer is a non-malignant inherited or acquired marrow disorder, and wherein the non-malignant inherited or acquired marrow disorder is selected from sickle anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, or congenital thrombocytopenia.

84. The method of any one of claims 75-83, which further comprises performing hematopoietic cell transplantation to the subject after the administering.

85. The method of claim 84, wherein the hematopoietic cell transplantation comprises transplantation to the subject of hematopoietic stem cells and/or hematopoietic progenitor cells.

86. The method of claim 84 or 85, wherein the performing of the hematopoietic cell transplantation occurs 5 days to 5 weeks after the administering.

87. The method of claim 86, wherein the performing of the hematopoietic cell transplantation occurs about 2 to 3 weeks after the administering.

88. The method of any one of claims 69-87, wherein the therapeutically effective amount is an amount of the antibody or fragment from about 0.01 mg/kg to about 2 mg/kg.

89. The method of claim 88, wherein the therapeutically effective amount is an amount of the antibody or fragment from about 0.1 mg/kg to about 0.3 mg/kg.

90. The method of any one of claims 69-89, wherein the administering is once a single dose.

91. The method of any one of claims 69-89, wherein the administering is every 1-14 days for about 1 to 4 weeks.

92. The method of any one of claims 69-89, wherein the administering is every 3-7 days for 2 to 3 weeks.

93. The method of any one of claims 69-92, wherein the administering is intravenous administration.

94. The method of claim 93, wherein the intravenous administration is by infusion into the subject.

95. The method of any one of claims 69-94, which further comprises administration of a checkpoint inhibitor.

96. The method of claim 95, wherein the checkpoint inhibitor is an anti-PD1 antagonist, an anti-PD-L1 antagonist and/or an anti-CTLA4 antagonist.

97. The method of claim 96, wherein the checkpoint inhibitor is an anti-PD1 antibody.

98. The method of any one of claims 95-97, wherein the administering is concomitant with or after the administration of the checkpoint inhibitor.

99. The method of any one of claims 69-98, wherein the subject is a human.
